SEQUENCE LISTING (91 sequences)

SEQ ID NO: 1 - DNA, 33 bp, synthetic construct
  source 1..33: mol_type = other DNA
catactctagaagcagatcatcgtggtgtcttg

SEQ ID NO: 2 - DNA, 31 bp, synthetic construct
  source 1..31: mol_type = other DNA
atcaggatcccgcggtctcggtgaccagctc

SEQ ID NO: 3 - DNA, 30 bp, synthetic construct
  source 1..30: mol_type = other DNA
tcatgagctccagaagacgctggtgctgac

SEQ ID NO: 4 - DNA, 30 bp, synthetic construct
  source 1..30: mol_type = other DNA
gactctagacatagctggtctccatcgctc

SEQ ID NO: 5 - DNA, 35 bp, synthetic construct
  source 1..35: mol_type = other DNA
tagactactagtcgtttacagctccgatccgaatg

SEQ ID NO: 6 - DNA, 33 bp, synthetic construct
  source 1..33: mol_type = other DNA
atactaggatccgtcggcttcaggacatggtcg

SEQ ID NO: 7 - DNA, 30 bp, synthetic construct
  source 1..30: mol_type = other DNA
cagctctagaatcatcagacaaggcgcgac

SEQ ID NO: 8 - DNA, 34 bp, synthetic construct
  source 1..34: mol_type = other DNA
tcatggatccattggcaagcgcatcacccggacc

SEQ ID NO: 9 - DNA, 36 bp, synthetic construct
  source 1..36: mol_type = other DNA
catatgactagtgagtgtccttcagcttctccagga

SEQ ID NO: 10 - DNA, 33 bp, synthetic construct
  source 1..33: mol_type = other DNA
catatgggatccgaatcaacactacagtccggt

SEQ ID NO: 11 - DNA, 35 bp, synthetic construct
  source 1..35: mol_type = other DNA
catatgggatcctgaacgacgcgcggcggcgaagc

SEQ ID NO: 12 - DNA, 35 bp, synthetic construct
  source 1..35: mol_type = other DNA
catatgcccgggacggtgagcaccgaattcgcctg

SEQ ID NO: 13 - DNA, 36 bp, synthetic construct
  source 1..36: mol_type = other DNA
catatggcggccgccgcgtgtcgtctcagcattgcg

SEQ ID NO: 14 - DNA, 33 bp, synthetic construct
  source 1..33: mol_type = other DNA
catatgggatcccatcacctcgtcgcggccgtc

SEQ ID NO: 15 - DNA, 35 bp, synthetic construct
  source 1..35: mol_type = other DNA
catatgggatcctgagcggtctgcggcaacgccgc

SEQ ID NO: 16 - DNA, 34 bp, synthetic construct
  source 1..34: mol_type = other DNA
catatgctgcagtggccgacgacaccaacgagct

SEQ ID NO: 17 - DNA, 40 bp, synthetic construct
  source 1..40: mol_type = other DNA
gctatatctagaagcgcaacgagagcttcgacattctgcc

SEQ ID NO: 18 - DNA, 35 bp, synthetic construct
  source 1..35: mol_type = other DNA
atatatggatccgcgtcgagcttctcgatcatcgc

SEQ ID NO: 19 - DNA, 33 bp, synthetic construct
  source 1..33: mol_type = other DNA
atatatggatcccaagctgcgcacatcatccca

SEQ ID NO: 20 - DNA, 33 bp, synthetic construct
  source 1..33: mol_type = other DNA
gcatatctcgagtctgatcatggagccggcttg

SEQ ID NO: 21 - DNA, 17 bp, synthetic construct
  source 1..17: mol_type = other DNA
gagaacactgtggtgct

SEQ ID NO: 22 - DNA, 22 bp, synthetic construct
  source 1..22: mol_type = other DNA
gagtgctgacctgagcgaatag

SEQ ID NO: 23 - DNA, 24 bp, synthetic construct
  source 1..24: mol_type = other DNA
cacttggtcgcgtcgatcacatag

SEQ ID NO: 24 - DNA, 21 bp, synthetic construct
  source 1..21: mol_type = other DNA
cgttcgcacttccggatggac

SEQ ID NO: 25 - DNA, 24 bp, synthetic construct
  source 1..24: mol_type = other DNA
cttgatggtctggttgccgccgac

SEQ ID NO: 26 - DNA, 24 bp, synthetic construct
  source 1..24: mol_type = other DNA
gaaccagctcgcgatccatctcag

SEQ ID NO: 27 - DNA, 27 bp, synthetic construct
  source 1..27: mol_type = other DNA
gccacgatgtagacttcctgtgccttg

SEQ ID NO: 28 - DNA, 25 bp, synthetic construct
  source 1..25: mol_type = other DNA
gagcaccttctgggcgagcacgatc

SEQ ID NO: 29 - DNA, 23 bp, synthetic construct
  source 1..23: mol_type = other DNA
atgccgatggcccaaaattcccg

SEQ ID NO: 30 - DNA, 21 bp, synthetic construct
  source 1..21: mol_type = other DNA
ctcgcaacaatcgtcgcactc

SEQ ID NO: 31 - DNA, 18 bp, synthetic construct
  source 1..18: mol_type = other DNA
aaggattggcctataccg

SEQ ID NO: 32 - DNA, 25 bp, synthetic construct
  source 1..25: mol_type = other DNA
cttccagaacgaaatcatgcagctc

SEQ ID NO: 33 - DNA, 20 bp, synthetic construct
  source 1..20: mol_type = other DNA
ggtggcgtcggaattccagt

SEQ ID NO: 34 - DNA, 19 bp, synthetic construct
  source 1..19: mol_type = other DNA
atccgcggcttgagcaagg

SEQ ID NO: 35 - DNA, 18 bp, synthetic construct
  source 1..18: mol_type = other DNA
ttggcatcccattccacg

SEQ ID NO: 36 - DNA, 23 bp, synthetic construct
  source 1..23: mol_type = other DNA
gtcaaacttcgccctcaccaatc

SEQ ID NO: 37 - DNA, 18 bp, synthetic construct
  source 1..18: mol_type = other DNA
cgaggcaatagccgaccg

SEQ ID NO: 38 - DNA, 20 bp, synthetic construct
  source 1..20: mol_type = other DNA
tcacttcgacaagtcgtgcc

SEQ ID NO: 39 - DNA, 20 bp, synthetic construct
  source 1..20: mol_type = other DNA
taatcgtgacgatggtcacc

SEQ ID NO: 40 - DNA, 20 bp, synthetic construct
  source 1..20: mol_type = other DNA
aggtccacagcttcaacgag

SEQ ID NO: 41 - DNA, 20 bp, synthetic construct
  source 1..20: mol_type = other DNA
gtgtcgatgccgttgaggat

SEQ ID NO: 42 - DNA, 30 bp, synthetic construct
  source 1..30: mol_type = other DNA
atcgaattccgctagcttcacgctgccgca

SEQ ID NO: 43 - DNA, 30 bp, synthetic construct
  source 1..30: mol_type = other DNA
gacccggcgtttgcggcgaccacggcgttc

SEQ ID NO: 44 - DNA, 30 bp, synthetic construct
  source 1..30: mol_type = other DNA
gtcgcgcatcaccgccgccttcggctacgg

SEQ ID NO: 45 - DNA, 30 bp, synthetic construct
  source 1..30: mol_type = other DNA
actcgtatatcggcccggaagcgacccagg

SEQ ID NO: 46 - DNA, 30 bp, synthetic construct
  source 1..30: mol_type = other DNA
gaccgcatctaaatgcatgcaggatgagga

SEQ ID NO: 47 - DNA, 30 bp, synthetic construct
  source 1..30: mol_type = other DNA
ggacgccgccgtgaaggccggcgcgccgaa

SEQ ID NO: 48 - DNA, 30 bp, synthetic construct
  source 1..30: mol_type = other DNA
tgcagtccgtcgagaagtcggagctgttca

SEQ ID NO: 49 - DNA, 30 bp, synthetic construct
  source 1..30: mol_type = other DNA
ctgttcaagctgggctacgtcaacaagatc

SEQ ID NO: 50 - DNA, 30 bp, synthetic construct
  source 1..30: mol_type = other DNA
ccacgcgatcgaggcgtatgtgtcggtgat

SEQ ID NO: 51 - DNA, 30 bp, synthetic construct
  source 1..30: mol_type = other DNA
gaacgccgtggtcgccgcaaacgccgggtc

SEQ ID NO: 52 - DNA, 30 bp, synthetic construct
  source 1..30: mol_type = other DNA
ccgtagccgaaggcggcggtgatgcgcgac

SEQ ID NO: 53 - DNA, 30 bp, synthetic construct
  source 1..30: mol_type = other DNA
cctgggtcgcttccgggccgatatacgagt

SEQ ID NO: 54 - DNA, 30 bp, synthetic construct
  source 1..30: mol_type = other DNA
tcctcatcctgcatgcatttagatgcggtc

SEQ ID NO: 55 - DNA, 30 bp, synthetic construct
  source 1..30: mol_type = other DNA
ttcggcgcgccggccttcacggcggcgtcc

SEQ ID NO: 56 - DNA, 30 bp, synthetic construct
  source 1..30: mol_type = other DNA
tgaacagctccgacttctcgacggactgca

SEQ ID NO: 57 - DNA, 30 bp, synthetic construct
  source 1..30: mol_type = other DNA
gatcttgttgacgtagcccagcttgaacag

SEQ ID NO: 58 - DNA, 30 bp, synthetic construct
  source 1..30: mol_type = other DNA
atcaccgacacatacgcctcgatcgcgtgg

SEQ ID NO: 59 - DNA, 30 bp, synthetic construct
  source 1..30: mol_type = other DNA
cgatcgatcgatcgatctgcagctccaaaa

SEQ ID NO: 60 - DNA, 30 bp, synthetic construct
  source 1..30: mol_type = other DNA
tctacaacaccctcgacaagatgagcgagc

SEQ ID NO: 61 - DNA, 30 bp, synthetic construct; identical to SEQ ID NO: 59
  source 1..30: mol_type = other DNA

SEQ ID NO: 62 - DNA, 27 bp, synthetic construct
  source 1..27: mol_type = other DNA
ccgagtctagacgtttcatatgtccgc

SEQ ID NO: 63 - DNA, 27 bp, synthetic construct
  source 1..27: mol_type = other DNA
gcgcgaattcttagatgcggtcgaagc

SEQ ID NO: 64 - DNA, 37 bp, synthetic construct
  source 1..37: mol_type = other DNA
gcatgaattcaggatgaggatcgtttcgcatgaaggt

SEQ ID NO: 65 - DNA, 35 bp, synthetic construct
  source 1..35: mol_type = other DNA
gttaggtaccgatcgatcgatccatctgcagctcc

SEQ ID NO: 66 - DNA, 37 bp, synthetic construct
  source 1..37: mol_type = other DNA
atcggcaagcttctaaccaggagatgtccatgtcgga

SEQ ID NO: 67 - DNA, 42 bp, synthetic construct
  source 1..42: mol_type = other DNA
atatctgcagtcaaaccatgtattggccgccgttgatggtga

SEQ ID NO: 68 - DNA, 37 bp, synthetic construct
  source 1..37: mol_type = other DNA
gcgaggacaagccaatcgccaccctcaccacccgcat

SEQ ID NO: 69 - DNA, 39 bp, synthetic construct
  source 1..39: mol_type = other DNA
gcctactcgtatatcggcccggaagcgacccaggccctc

SEQ ID NO: 70 - DNA, 20 bp, synthetic construct
  source 1..20: mol_type = other DNA
agtcacggccgaggtggaag

SEQ ID NO: 71 - DNA, 18 bp, synthetic construct
  source 1..18: mol_type = other DNA
gcgtcctgaagccgttcg

SEQ ID NO: 72 - DNA, 40 bp, synthetic construct
  source 1..40: mol_type = other DNA
cggcatcatcgaccacgacgacagcctcggcatcaccaag

SEQ ID NO: 73 - DNA, 38 bp, synthetic construct
  source 1..38: mol_type = other DNA
cgggccatacctcgtcgctgtatatcgacagccagaac

SEQ ID NO: 74 - DNA, 19 bp, synthetic construct
  source 1..19: mol_type = other DNA
tggtgctgatgaccgccaa

SEQ ID NO: 75 - DNA, 20 bp, synthetic construct
  source 1..20: mol_type = other DNA
tgggacgtgagttcgttcga

SEQ ID NO: 76 - DNA, 20 bp, synthetic construct
  source 1..20: mol_type = other DNA
cctttaagctgccggtgttc

SEQ ID NO: 77 - DNA, 18 bp, synthetic construct
  source 1..18: mol_type = other DNA
gtggtgagggtggcgatt

SEQ ID NO: 78 - DNA, 20 bp, synthetic construct
  source 1..20: mol_type = other DNA
gcgcaacaatatctgcctga

SEQ ID NO: 79 - DNA, 20 bp, synthetic construct
  source 1..20: mol_type = other DNA
tgatgcgcttcttggtgtac

SEQ ID NO: 80 - DNA, 20 bp, synthetic construct
  source 1..20: mol_type = other DNA
acctgctgtacgagtatccg

SEQ ID NO: 81 - DNA, 20 bp, synthetic construct
  source 1..20: mol_type = other DNA
cagcttcgggaaattgcaga

SEQ ID NO: 82 - DNA, 20 bp, synthetic construct
  source 1..20: mol_type = other DNA
ggatgccttcaacggttacc

SEQ ID NO: 83 - DNA, 20 bp, synthetic construct
  source 1..20: mol_type = other DNA
cgcgaattcatcctgctgat

SEQ ID NO: 84 - DNA, 20 bp, synthetic construct
  source 1..20: mol_type = other DNA
tttgaatttctccgctgccg

SEQ ID NO: 85 - DNA, 20 bp, synthetic construct
  source 1..20: mol_type = other DNA
ggtatcggtgcagcaatcag

SEQ ID NO: 86 - DNA, 19 bp, synthetic construct
  source 1..19: mol_type = other DNA
atcggccagatcaaggaac

SEQ ID NO: 87 - DNA, 20 bp, synthetic construct
  source 1..20: mol_type = other DNA
gaattcgacctgcttgaacg

SEQ ID NO: 88 - DNA, 20 bp, synthetic construct
  source 1..20: mol_type = other DNA
ggagatctgcaaggttctcg

SEQ ID NO: 89 - DNA, 20 bp, synthetic construct
  source 1..20: mol_type = other DNA
ctcgtcctgcagttcattca

SEQ ID NO: 90 - DNA, 20 bp, synthetic construct
  source 1..20: mol_type = other DNA
ctcgtcctgcagttcattca

SEQ ID NO: 91 - DNA, 20 bp, synthetic construct
  source 1..20: mol_type = other DNA
agacaatcggctgctctgat

(19) United States
(12) Patent Application Publication
(10) Pub. No.: US 2023/0242947 A1
(43) Pub. Date: Aug. 3, 2023

(54) GENETICALLY ENGINEERED RHODOPSEUDOMONAS PALUSTRIS

(71) Applicants: Arpita Bose, St. Louis, MO (US); Wei Bai, St. Louis, MO (US)

(72) Inventors: Arpita Bose, St. Louis, MO (US); Wei Bai, St. Louis, MO (US)

(73) Assignee: Washington University, St. Louis, MO (US)

(21) Appl. No.: 18/046,695

(22) Filed: Oct. 14, 2022

Related U.S. Application Data
(60) Provisional application No. 63/255,590, filed on Oct. 14, 2021.

(51) Int. Cl. C12P 7/625; C12N 15/52; C12N 15/74; C12N 9/12

(52) U.S. Cl. CPC C12P 7/625; C12N 15/52; C12N 15/74; C12N 9/1241; C12Y 401/01039; C12Y 402/01027; C12Y 103/01086; C12Y 102/01057; C12Y 101/05; C12N 2800/101; C12N 2820/002

(57) ABSTRACT

Among the various aspects of the present disclosure is the provision of a genetically engineered transgenic microorganisms Rhodopseudomonas palustris and methods of making and using the same.

### CROSS-REFERENCE TO RELATED APPLICATIONS

This application claims priority from U.S. Provisional Application Ser. No. 63/255,590 filed on 14 Oct. 2021, which is incorporated herein by reference in its entirety.

### STATEMENT REGARDING FEDERALLY SPONSORED RESEARCH OR DEVELOPMENT

This invention was made with government support under 2021822 awarded by the National Science Foundation, DE-SC0014613 awarded by the U.S. Department of Energy and W911NF-18-1-0037 awarded by the ARMY Research Laboratory. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name 019598-US-NP_Sequence_Listing_ST26.xml created on 7 Oct. 2022; 81,542 bytes). The subject matter of the Sequence Listing is incorporated herein by reference in its entirety.

### FIELD

The present disclosure generally relates to fuel production using a genetically modified anoxygenic photoautotroph.

### SUMMARY

Among the various aspects of the present disclosure is the provision of a transgenic microorganisms and methods of making and using the same.

An aspect of the present disclosure provides for a genetic tool or a method for integrating a target gene into a genome of Rhodopseudomonas palustris TIE-1 (TIE-1), the tool or method comprising: providing a phage integration system (phage recombination) to integrate the target gene; providing a φC31 recombinase system comprising an attB site, an attP site, and a φC31 integrase; wherein in the presence of the φC31 integrase, the attB site and the attP site recombine with each other resulting in insertion of a whole plasmid into the genome; wherein the attB site is introduced into the genome of TIE-1, and/or wherein the attP site is introduced on a plasmid (e.g., a suicide plasmid, optionally with a constitutively expressed mcherry gene). In some embodiments, the expression of φC31 integrase is achieved by (i) a plasmid-based system, where the integrase is introduced into TIE-1 by a plasmid; or (ii) a genome-based system, where the integrase is integrated into the TIE-1 genome. In some embodiments, φC31 recombinase system comprises inducible promoter (P_{lac}) and a strong constitutive promoter (Pₐₚₕₗₗ). In some embodiments, the φC31 integrase is achieved via: a) Pₐₚₕₗₗ-driven φC31 integrase on a suicide plasmid; b) P_{lac}-driven φC31 integrase on a self-replicating plasmid; c) Pₐₚₕₗₗ-driven φC31 integrase on TIE-1 genome; or d) P_{lac}-driven φC31 integrase on TIE-1 genome. Another aspect of the present disclosure provides for a transgenic microorganism comprising an artificial DNA construct comprising, as operably associated components in the 5′ to 3′ direction of transcription: (i) a promoter functional in the microorganism; (ii) (a) a first polynucleotide comprising a nucleotide sequence encoding a first polypeptide having a crotonase activity; (b) a second polynucleotide comprising a nucleotide sequence encoding a second polypeptide having a crotonyl-CoA reductase activity; (c) a third polynucleotide comprising a nucleotide sequence encoding a third polypeptide having a butyraldehyde dehydrogenase/butanol dehydrogenase activity; and optionally, (d) a fourth polynucleotide comprising a nucleotide sequence encoding a fourth polypeptide having a thiolase/Acetyl/CoA synthase activity; and/or (e) a fifth polynucleotide comprising a nucleotide sequence encoding a fifth polypeptide having a 3-hydroxybutyryl-CoA-dehydrogenase activity; and/or (iii) a transcriptional termination sequence. In some embodiments, the artificial DNA construct comprises a sixth polynucleotide comprising a nucleotide sequence encoding a sixth polypeptide having a φC31 integrase activity. In some embodiments, the genetic tool, method, or transgenic microorganism of any one of the preceding aspects or embodiments, comprises integrating one or more copies of RuBisCo. In some embodiments, the genetic tool, method, or transgenic microorganism of any one of the preceding aspects or embodiments, wherein the introduced genes increase bioplastic production. Yet another aspect of the present disclosure provides for a wild-type TIE-1 or transgenic microorganism according to any one of the preceding aspects or embodiments lacking electron-consuming (nitrogen-fixing) pathways. Yet another aspect of the present disclosure provides for a TIE-1 mutant carrying one or more copies of a native, artificial native (foreign) phaA, or phaB, or artificial non-native (foreign) phaJ, ter, or adhE2. Yet another aspect of the present disclosure provides for a 3-gene cassette comprising phaJ, ter, and adhE2. Yet another aspect of the present disclosure provides for a 5-gene cassette comprising the 3-gene cassette, and a copy of the phaA-phaB operon from TIE-1. In some embodiments, the phaJ gene is isolated from Aeromonas caviae. In some embodiments, the ter gene is isolated from Euflena gracilis and it is unable to catalyze the reverse oxidation of butyryl-CoA. In some embodiments, the adhE2 gene is isolated from C. acetobutylicum and the enzyme encodes for specifically catalyzes the reduction of the butyryl-CoA. In some embodiments, the phaJ-ter-adhE2 cassette is inserted into plasmid pRhokS-2, resulting in pAB675. In some embodiments, the PhaA and phaB were amplified as an operon from the R. palustris TIE-1 genome. In some embodiments, the phaA-phaB cassette was then cloned into pAB675 to obtain pAB744. In some embodiments, wherein, upon obtaining mutants and plasmids, either the 3-gene or the 5-gene was conjugated into WT TIE-1 or the mutants, using mating the strain E. coli S17-1/λ. In some embodiments, the methods, constructs, or microorganisms of any one of the preceding aspects or embodiments comprises phaA (native or foreign), phaB (native or foreign), phaJ (foreign), ter, and adhE2. In some embodiments, the transgenic TIE-1 is a nitrogenase knockout mutant. In some embodiments, the TIE-1 can produce n-butanol using different carbon sources (organic acids, CO₂), electron sources [H₂, Fe(II), a poised electrode], and nitrogen sources (NH⁺, N₂) (e.g., photoelectroautotrophy using light, electricity, and CO₂). In some embodiments, each gene has its own promoter. In some embodiments, the methods, constructs, or microorganisms of any one of the preceding aspects or embodiments, comprises increasing intracellular iron. In some embodiments, the additional pathways that consume electrons are deleted. In some embodiments, the transgenic microorganism overproduces n-butanol compared to a microorganism not comprising the artificial DNA construct. In some embodiments, an n-butanol product accumulates within the microorganism. Yet another aspect of the present disclosure provides for a transgenic microorganism having thiolase/Acetyl/CoA synthase activity (native or foreign), 3-hydroxybutyryl-CoA-dehydrogenase activity (native or foreign), crotonase activity, crotonyl-CoA reductase activity, butyraldehyde dehydrogenase/butanol dehydrogenase activity, capable of producing an n-butanol product prepared by a process comprising the steps of: (i) providing the transgenic microorganism comprising a native, an artificial native, or an artificial non-native/foreign polynucleotide capable of encoding a polypeptide having thiolase/Acetyl/CoA synthase and 3-hydroxybutyryl-CoA-dehydrogenase enzymatic activity; an artificial (non-native/foreign) polynucleotide capable of encoding a polypeptide having crotonase enzymatic activity; an artificial (non-native/foreign) polynucleotide capable of encoding a polypeptide having crotonyl-CoA reductase enzymatic activity; and/or an artificial (non-native/foreign) polynucleotide capable of encoding a polypeptide having butyraldehyde dehydrogenase/butanol dehydrogenase enzymatic activity; (ii) cultivating the microorganism; and/or (iii) isolating an accumulated n-butanol product. In some embodiments, the transgenic microorganism is capable of producing an n-butanol product prepared by a process further comprising the steps of: providing the transgenic microorganism and providing a feedstock, wherein the transgenic microorganism comprises a non-native or native copy of phaA, a non-native or native copy of phaB, a non-native copy of phaJ, a non-native copy of ter, and a non-native copy of adhe2 under a constitutive or inducible promoter. In some embodiments, the feedstock comprises renewable energy, natural resources, wind power, solar power, hybrid bioelectrochemical platforms, CO₂, solar panel-generated electricity, and light.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1B shows an exemplary embodiment of the n-butanol synthesis pathway and cassette design used for n-butanol production in Rhodopseudomonas palustris TIE-1 in accordance with the present disclosure. FIG. 1A is a schematic showing the n-butanol biosynthesis pathway involving five genes. The enzymes encoded by each gene and the reactions catalyzed by these enzymes are shown in dark blue. Two major byproducts (acetone and ethanol) are shown in dark red. NADH, nicotinamide adenine dinucleotide; NADPH, nicotinamide adenine dinucleotide phosphate. FIG. 1B is a schematic depicting cassette design. The 3-gene cassette relies on phaA and phaB on the genome of TIE-1 for the first two steps of n-butanol synthesis. Here, only 3-genes (phaJ, ter, and adhE2) were introduced on a plasmid under a constitutive promoter PaphII. The 5-gene cassette has all five genes (phaA, phaB, phaJ, ter, and adhE2) on the plasmid under a constitutive promoter Pₐₚₕₗₗ.

FIG. 2A is a box plot showing glycogen content of WT and Gly mutant grown with NH₄⁺ or N₂ as a nitrogen source. Data are from n=3 of independent experiments. 3-Hydroxybutyrate was used as a carbon source for all the growth conditions. WT: wild type; Gly: glycogen synthase knockout.

FIG. 2B-FIG. 2E is a series of TEM micrographs of polyhydroxybutyrate granulate (bright spots in the figures) in WT and Phb mutant. FIG. 2B depicts WT with N². FIG. 2C depicts WT with NH₄⁺. FIG. 2D depicts Phb with N₂. FIG. 2E depicts Phb mutant with NH₄⁺. 3-Hydroxybutyrate was used as a carbon source for all the growth conditions. Phb: 3-hydroxybutyrate polymerase knockout.

FIG. 3 is a schematic depicting the relationship between polyhydroxybutyrate, glycogen, and n-butanol biosynthesis pathways and Calvin-Benson-Bassham cycle. Pink: n-butanol biosynthesis pathway, Green: Calvin-Benson-Bassham cycle, Blue: glycogen and polyhydroxybutyrate biosynthesis.

FIG. 4 is a box plot showing the NADH/NAD⁺ ratio of nitrogenase knockout and wild-type strain. Data are from n=3 of independent experiments. WT: wild type, Nif: nitrogenase knockout, 3Hy: using 3-hydroxybutyrate as carbon and electron source, H₂: using hydrogen as an electron source, CO₂ as carbon source. All growth used NH₄⁺ as the nitrogen source.

FIG. 5A-FIG. 5B shows an exemplary embodiment of major metabolisms used for n-butanol production in Rhodopseudomonas palustris TIE-1 (TIE-1) in accordance with the present disclosure. FIG. 5A is a schematic depicting photoheterotrophy wherein TIE-1 uses organic acids as carbon and electron source, light as an energy source, and ammonium (NH₄⁺) or dinitrogen gas (N₂) as a nitrogen source. FIG. 5B is a schematic depicting photoautotrophy wherein TIE-1 uses carbon dioxide as carbon source, hydrogen (H₂), ferrous iron [Fe(II)], or poised electrode as an electron source, light as an energy source, and NH₄⁺ or N₂ as a nitrogen source.

FIG. 6 is a schematic depicting substrates and by-products of n-butanol biosynthesis. Black: substrates; Red: n-butanol synthesis intermediates; Green: in-cell by-products; Blue: secreted by-products.

FIG. 7A-FIG. 7D is an exemplary embodiment showing the nitrogenase double mutant (Nif) produced the highest amount of n-butanol in the presence of 3-hydroxybutyrate in accordance with the present disclosure. The concentration of n-butanol in mg/L was measured when TIE-1 was cultured with ammonium (NH₄⁺, red) or dinitrogen gas (N₂, blue) and various carbon and electron sources. FIG. 7A is a box plot showing n-butanol concentration using acetate (photoheterotrophy). FIG. 7B is a box plot showing n-butanol concentration using 3-hydroxybutyrate (photoheterotrophy). FIG. 7C is a box plot showing n-butanol concentration using hydrogen (H₂) (photoautotrophy). FIG. 7D is a box plot showing n-butanol concentration using ferrous iron [Fe(II)] (photoautotrophy). CO₂ was present in all conditions. Data are from n=3 of independent experiments. Boxes that only have two biological replicates are indicated by ‘*’. WT-3: wild type with 3-gene cassette; WT-5: wild type with 5-gene cassette; Nif-3: nitrogenase knockout with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette, n.d. (non-detectable).

FIG. 8A-FIG. 8D is an exemplary embodiment showing 3-Hydroxybutyrate resulted in the highest n-butanol productivity in accordance with the present disclosure. n-butanol productivity was measured when TIE-1 was cultured with ammonium (NH₄⁺, red) or dinitrogen gas (N₂, blue) and various carbon and electron sources. FIG. 8A is a box plot showing n-butanol productivity using acetate (photoheterotrophy). FIG. 8B is a box plot showing n-butanol productivity using 3-hydroxybutyrate (photoheterotrophy). FIG. 8C is a box plot showing n-butanol productivity using hydrogen (H₂) (photoautotrophy). FIG. 8D is a box plot showing n-butanol productivity using ferrous iron [Fe(II)] (photoautotrophy). CO₂ was present in all conditions. Data are from n=3 of independent experiments. Boxes with data from n=2 independent experiments are indicated by ‘*’. WT-3: wild type with 3-gene cassette; WT-5: wild type with 5-gene cassette; Nif-3: nitrogenase knockout with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette, n.d. (non-detectable).

FIG. 9A-FIG. 9D is an exemplary embodiment showing high n-butanol production correlates to low acetone production amongst TIE-1 mutants in accordance with the present disclosure. The concentration of acetone in mg/L was measured when TIE-1 was cultured with ammonium (NH₄⁺ red) or dinitrogen gas (N₂, blue) and various carbon and electron sources. FIG. 9A is a box plot showing acetone concentration using acetate (photoheterotrophy). FIG. 9B is a box plot showing acetone concentration using 3-hydroxybutyrate (photoheterotrophy). FIG. 9C is a box plot showing acetone concentration using hydrogen (H₂) (photoautotrophy). FIG. 9D is a box plot showing acetone concentration using ferrous iron [Fe(II)] (photoautotrophy). CO₂ was present in all conditions. Data are from n=3 of independent experiments. WT-3: wild type with 3-gene cassette; WT-5: wild type with 5-gene cassette; Nif-3: nitrogenase knockout with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette, n.d. (non-detectable).

FIG. 10A-FIG. 10B is a series of box plots showing electron donor consumption when TIE-1 was cultured with ammonium (NH₄⁺ red) or dinitrogen gas (N₂, blue) and various carbon and electron sources. FIG. 10A shows consumption of the electron donor acetate (photoheterotrophy). FIG. 10B shows consumption of the electron donor 3-hydroxybutyrate (photoheterotrophy). Data are from n=3 of independent experiments. Carbon dioxide was present in all conditions. CO₂: carbon dioxide; WT-3: wild type with 3-gene cassette; WT-5: wild type with 5-gene cassette; Nif-3: nitrogenase knockout t with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette.

FIG. 11A-FIG. 11H is an exemplary embodiment showing the nitrogenase double mutant (Nif) converts carbon to n-butanol more efficiently in accordance with the present disclosure. CO₂ consumption (positive value)/production (negative value) or carbon conversion efficiency to n-butanol was measured using various carbon and electron sources. FIG. 11A is a box plot showing CO₂ consumption using acetate (photoheterotrophy). FIG. 11B is a box plot showing CO₂ consumption using 3-hydroxybutyrate (photoheterotrophy). FIG. 11C is a box plot showing CO₂ consumption using hydrogen (H₂) (photoautotrophy). FIG. 11D is a box plot showing CO₂ consumption using ferrous iron [Fe(II)] (photoautotrophy). FIG. 11E is a box plot showing carbon conversion efficiency using acetate (photoheterotrophy). FIG. 11F is a box plot showing carbon conversion efficiency using 3-hydroxybutyrate (photoheterotrophy). FIG. 11G is a box plot showing carbon conversion efficiency using hydrogen (H₂) (photoautotrophy). FIG. 11H is a box plot showing carbon conversion efficiency using ferrous iron [Fe(II)] (photoautotrophy). CO₂ was present in all conditions. Data are from n=3 of independent experiments. WT-3: wild type with 3-gene cassette; WT-5: wild type with 5-gene cassette; Nif-3: nitrogenase knockout with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette, n.d. non-detectable.

FIG. 12A-FIG. 12C is an exemplary embodiment showing substrate consumption and hydrogen production under photoheterotrophic conditions in accordance with the present disclosure. Electron donor consumption was measured when TIE-1 was cultured with ammonium (NH₄⁺, red) or dinitrogen gas (N₂, blue) and various carbon and electron sources. FIG. 12A sis a b consumption of the electron donor hydrogen (H₂) (photoautotrophy). FIG. 12B shows consumption of the electron donor ferrous iron (Fe(II)) (photoautotrophy). Data are from n=3 of independent experiments. FIG. 12C shows H₂ production of WT-3/WT-5 and Gly-3/Gly-5 mutant under photoheterotrophic conditions. Data are from n=3 of independent experiments. Carbon dioxide was present in all conditions. CO₂: carbon dioxide; WT-3: wild type with 3-gene cassette; WT-5: wild type with 5-gene cassette; Nif-3: nitrogenase knockout t with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette.

FIG. 13A-FIG. 13D is an exemplary embodiment showing the nitrogenase double mutant (Nif) converts electrons to n-butanol more efficiently in accordance with the present disclosure. The electron conversion efficiency towards n-butanol (%) was measured when TIE-1 was cultured with ammonium (NH₄⁺ red) or dinitrogen gas (N₂, blue) and various carbon and electron sources. FIG. 13A is a box plot showing the electron conversion efficiency using acetate (photoheterotrophy). FIG. 13B is a box plot showing the electron conversion efficiency using 3-hydroxybutyrate (photoheterotrophy). FIG. 13C is a box plot showing the electron conversion efficiency using hydrogen (H₂) (photoautotrophy). FIG. 13D is a box plot showing the electron conversion efficiency using ferrous iron [Fe(II)] (photoautotrophy). CO₂ was present in all conditions. Data are from n=3 of independent experiments. Boxes that only have two biological replicates are indicated by ‘*’. WT-3: wild type with 3-gene cassette; WT-5: wild type with 5-gene cassette; Nif-3: nitrogenase knockout t with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette, n.d. (non-detectable).

FIG. 14A-FIG. 14I is an exemplary embodiment showing using hydrogen as an electron donor resulted in low electron flow in byproduct generation in accordance with the present disclosure. Electron consumption of n-butanol/acetone/H₂/CO₂/biomass synthesis was measured under various incubation conditions. FIG. 14A is a box plot showing electron consumption using acetate and NH₄⁺ (photoheterotrophy). FIG. 14B is a box plot showing electron consumption using acetate and N₂ (photoheterotrophy). FIG. 14C is a box plot showing electron consumption using 3-hydroxybutyrate and NH₄ (photoheterotrophy). FIG. 14D is a box plot showing electron consumption using 3-hydroxybutyrate and N₂ (photoheterotrophy). FIG. 14E is a box plot showing electron consumption using hydrogen (H₂) and NH₄⁺ (photoautotrophy). FIG. 14F is a box plot showing electron consumption using hydrogen (H₂) and N₂ (photoautotrophy). FIG. 14G is a box plot showing electron consumption using ferrous iron [Fe(II)] and NH₄⁺ (photoautotrophy). FIG. 14H is a box plot showing electron consumption using ferrous iron [Fe(II)] and N₂ (photoautotrophy). FIG. 14I is a table showing electron consumption of n-butanol synthesis under various incubation conditions. CO₂ was present in all conditions. Data are from n=3 of independent experiments. WT-3: wild type with 3-gene cassette; WT-5: wild type with 5-gene cassette; Nif-3: nitrogenase knockout t with 3-gene cassette; Nif-5: nitrogenase knockout with 5-gene cassette; Gly-3: glycogen synthase knockout with 3-gene cassette; Gly-5: glycogen synthase knockout with 5-gene cassette; Phb-3: hydroxybutyrate polymerase knockout with 3-gene cassette n.d. is non-detectable and n.a. is not available.

FIG. 15 is a schematic depicting setup of the three-electrodes configured sealed type bioelectrochemical cell (BEC) platform. Under photoelectroautotrophic conditions, TIE-1 gains electrons from a poised electrode, using light as an energy source and carbon dioxide as a carbon source. 1—electricity source 2-light source, 3—Purge inlet, 4—Reference electrode (Ag/AgCl in 3M KCl), 5—Counter electrode (Pt foil, 5 cm²), 6—Working electrode (Graphite rod, 3.2 cm²), 7—electrical wire DAQ—Data acquisition).

FIG. 16 is a box plot depicting electron uptake in an abiotic control in the presence of ammonium (NH₄⁺, red) or dinitrogen gas (N₂, blue) with various light and electricity sources. Data are from n=2 of independent experiments. SO: using electricity generated by a solar panel; HA: using halogen light as the light source. PO: using electricity from potentiostat as the electricity source; IR: using infrared light as the light source.

FIG. 17 is a box plot showing n-butanol production by the nitrogenase double mutant with the 5-gene cassette under photoelectroautotrophy. For all the platforms, either ammonium (NH₄⁺) or dinitrogen gas (N₂) was supplied. PO potentiostat, IR infrared light, HA halogen light, SO solar panel. Data are from n=2 of independent experiments.

FIG. 18A-FIG. 18B is an exemplary embodiment showing carbon consumption and electron uptake in each reactor setup in accordance with the present disclosure. FIG. 18A is a box plot showing carbon dioxide consumption and FIG. 18B is a box plot showing current uptake in the presence of ammonium (NH₄⁺ red) or dinitrogen gas (N₂, blue) with various light and electricity sources. For all the platforms, either ammonium (NH₄⁺) or dinitrogen gas (N₂) was supplied. Data are from n=2 of independent experiments. SO: using electricity generated by a solar panel; HA: using halogen light as the light source. PO: using electricity from potentiostat as the electricity source; IR: using infrared light as the light source.

FIG. 19A-FIG. 19C is an exemplary embodiment showing acetone production, carbon conversion efficiency, and electron conversion efficiency by the nitrogenase double mutant with the 5-gene cassette under photoelectroautotrophy in accordance with the present disclosure. FIG. 19A is a box plot showing acetone production. FIG. 19B is a box plot showing carbon conversion efficiency (CCE) towards n-butanol. FIG. 19C is a box plot showing electron conversion efficiency (ECE) towards n-butanol. For all the platforms, either ammonium (NH₄⁺) or dinitrogen gas (N₂) was supplied. PO potentiostat, IR infrared light, HA halogen light, SO solar panel. Data are from n=2 of independent experiments.

FIG. 20A-FIG. 20B is a series of box plots showing cell viability in each reactor setup. FIG. 20A shows the percentage of live cells and FIG. 20B shows the total number of live cells in the presence of ammonium (NH₄⁺ red) or dinitrogen gas (N₂, blue) with various light and electricity sources. Data are from n=2 of independent experiments. SO: using electricity generated by a solar panel; HA: using halogen light as the light source. PO: using electricity from potentiostat as the electricity source; IR: using infrared light as the light source.

FIG. 21 is a box plot showing electrical energy conversion efficiency (EECE) towards n-butanol by the nitrogenase double mutant with the 5-gene cassette under photoelectroautotrophy. For all the platforms, either ammonium (NH₄⁺) or dinitrogen gas (N₂) was supplied. PO potentiostat, IR infrared light, HA halogen light, SO solar panel. Data are from n=2 of independent experiments.

FIG. 22 is a box plot showing energy conversion efficiency from light. Data are from n=3 of independent experiments. SO: using electricity generated by a solar panel; HA: using halogen light as the light source. PO: using electricity from potentiostat as the electricity source; IR: using infrared light as the light source. NH₄⁺: ammonium; N₂: dinitrogen gas.

FIG. 23 is a box plot showing mRNA log₂ fold change of butanol synthesis genes (phaJ, ter, adhE2, phaA, and phaB). Fold change was calculated using Nif mutant with 5-gene on 3Hy-NH₄⁺ as the reference. The total expression level of each enzyme was measured. Accordingly, the different copies of the phaA and phaB genes were not distinguished. The fold changes were calculated using RT-qPCR with the comparative Cₜ method and clpX and recA were used as internal standards. Data are from n=3 of independent experiments. 3Hy-N₂: using 3-hydroxybutyrate as a major carbon/electron source and dinitrogen gas (N₂) as the nitrogen source. Fe—NH₄⁺: using carbon dioxide (CO₂) as a carbon source, ferrous iron as an electron source, and ammonium (NH₄⁺) as the nitrogen source. 3Hy-NH₄⁺: using 3-hydroxybutyrate as major carbon/electron source and NH₄⁺ as the nitrogen source. CO₂ was present in all conditions.

FIG. 24A-FIG. 24J shows an exemplary embodiment of PCR check for mutants in accordance with the present disclosure. FIG. 24A is a schematic depicting a view of two different primer sets. FIG. 24B is a table showing expected band sizes. FIG. 24C is an image showing the primer set 1 for nifA1 (Rpal_1624) to the left of the ladder and primer set 1 for nifA2 (Rpal_5113) to the right of the ladder for the Nif mutant. FIG. 24D is an image showing the primer set 2 for nifA1 (Rpal_1624) to the right of the ladder for the Nif mutant. FIG. 24E is an image showing primer set 2 for nifA2 (Rpal_5113) to the right of the ladder for the Nif mutant. FIG. 24F is an image showing primer set 1 for glgA (Rpal_0386) to the left of the ladder and primer set 2 for glgA (Rpal_0386) to the right of the ladder for the Gly mutant. FIG. 24G is an image showing primer set 1 for phaC1 (Rpal_2780) for the Phb mutant. FIG. 24H is an image showing primer set 1 for phaC2 (Rpal_4722) for the Phb mutant. FIG. 24I is an image showing primer set 2 for phaC1 (Rpal_2780) for the Phb mutant. FIG. 24J is an image showing primer set 2 for phaC2 (Rpal_4722) for the Phb mutant. Genomic DNA from WT or TIE-1 mutant was used as a PCR template. Depending on the primer set, either WT(W) or mutant (M) genomic DNA was used as the positive control (+). Autoclaved Mili Q water was used as negative control (−). L: Thermal Fisher 1 kb plus DNA ruler for FIG. 24C-FIG. 24F or GeneRuler 1 kb plus DNA ruler for FIG. 24G-FIG. 24J. M: mutant; W: Wild type; +: positive control; −: negative control.

FIG. 25A-FIG. 25H shows an exemplary embodiment of PCR checks of inoculum for n-butanol cassettes in accordance with the present disclosure. FIG. 25A is an image showing WT inoculum for photoheterotrophic and photoautotrophic conditions using hydrogen or ferrous iron as electron donor. S1: WT with 3-gene cassette, S2: Wild Type with 5-gene cassette. FIG. 25B is an image showing Nif mutant inoculum for photoheterotrophic and photoautotrophic conditions using hydrogen or ferrous iron as electron donor. S1: Nif mutant with 3-gene cassette; S2: Nif mutant with 5-gene cassette. FIG. 25C is an image showing Gly mutant inoculum for photoheterotrophic and photoautotrophic conditions using hydrogen or ferrous iron as electron donor. S1: Gly mutant with 3-gene cassette; S2: Gly mutant with 5-gene cassette. FIG. 25D is an image showing Phb mutant inoculum for photoheterotrophic and photoautotrophic conditions using hydrogen or ferrous iron as electron donor. S: Phb mutant with 3-gene cassette. FIG. 25E is an image showing inoculums of Nif mutant with 5-gene cassette inoculum for photoelectroautotrophy using electricity from the solar panel under halogen light. Lane S: Nif mutant with 5-gene cassette. FIG. 25F is an image showing inoculums of Nif mutant with 5-gene cassette inoculum for photoelectroautotrophy using electricity from the solar panel under infrared light. S: Nif mutant with 5-gene cassette. FIG. 25G is an image showing inoculums of Nif mutant with 5-gene cassette inoculum for photoelectroautotrophy using electricity from potentiostat under halogen light. S: Nif mutant with 5-gene cassette. FIG. 25H is an image showing inoculums of Nif mutant with 5-gene cassette inoculum for photoelectroautotrophy using electricity from potentiostat under infrared light. S: Nif mutant with 5-gene cassette. A 550 bp band is expected from both 3-gene and 5-gene cassettes. Miniprep from inoculum was used as a PCR template. A plasmid with either the 3-gene cassette or the 5-gene cassette was used as a positive control. Autoclaved Milli Q water was used as negative control. L:1 kb plus DNA ladder. S: sample; +: positive control; −: negative control.

FIG. 26A-FIG. 26B shows an exemplary embodiment of flow of two-step integration in accordance with the present disclosure. FIG. 26A is a schematic depicting the flow of generating a knockout mutant. FIG. 26B is a schematic depicting the flow of generating a knock-in mutant UP: upstream homologous arm, DN: down-stream homologous arm, Genr: gentamicin resistance, SacB: sucrose counter-selection marker, WT: wild type.

FIG. 27A-FIG. 27F is an exemplary embodiment of the phage integration system in accordance with the present disclosure. FIG. 27A is a schematic depicting the φC31 integrase mechanism. FIG. 27B is a schematic depicting the plasmid-based integration system. FIG. 27C is a schematic depicting the genome-based integration system. FIG. 27D is a graph showing the electroporation efficiency normalized by plasmid. FIG. 27E is a graph showing the CFU normalized by OD. FIG. 27F is a graph showing editing efficiency. P_{lac}: P_{lac} promoter, Pₐₚₕₗₗ, Pₐₚₕₗₗ promoter, mcherry: red fluorescent protein, gen^{R}: gentamicin resistance, p15A: the origin of replication, pBBR1: broad host origin of replication attP: attP site for φC31 integrase, attB: attB site for φC31 integrase, φC31: φC31 integrase, TE: transformation efficiency, PC: plasmid-based system with the constitutive promoter, PI: plasmid-based system with the inducible promoter, GI: genome-based system with the inducible promoter, CFU: colony forming units, OD: optical density.

### DETAILED DESCRIPTION

The present disclosure is based, at least in part, on the discovery that mutant TIE-1 can be used as a microbial chassis for carbon-neutral n-butanol bioproduction using sustainable, renewable, and abundant resources. As shown herein, a mutant lacking the nitrogen-fixing pathway produced the highest n-butanol synthesis. Coupled with novel hybrid bioelectrochemical platforms, this mutant produced n-butanol using CO₂, solar panel-generated electricity, and light, with high electrical energy conversion efficiency.

This disclosure provides for the use of mutants of TIE-1 (WT+3/VVT+5, Nif+3/Nif+5). Although the knockouts and the genes used in the cassettes described here have been previously used in other bacteria, it is presently believed that the combination of these has not been studied in TIE-1. It would not be trivial to introduce these modifications in an anoxygenic phototroph TIE-1. Furthermore, most of the organisms previously studied are chemoheterotrophs which would make the n-butanol production using these microbes carbon-positive, where the use of the TIE-1 in the present disclosure is carbon-neutral. Surprisingly, Nif+3/Nif+5 produced a higher amount of n-butanol than the WT+3/WT+5, even in the presence of NH₄⁺.

Also provided herein is a new genetic platform. As described herein tools have been developed for the genetic manipulation of TIE-1, which have not been genetically manipulated before.

The new platform described here was recently rated at a Technology Readiness Level of 4 which is the highest level of any of the organisms evaluated and ahead of older model organisms (Abel et al., bioRxiv preprint, doi: https://doi.org/10.1101/2020.12.07.414987, posted Dec. 9, 2020).

The disclosed process allows a conversion of electricity to biofuels with high electrical energy conversion efficiency. Thus, excess renewable (solar/wind, etc. generated) electricity can be stored as easily transported liquid fuel for later use. n-butanol is an advanced drop-in fuel that is compatable with the current engines and fuel system or the fuel distribution network.

Molecular Engineering

Genes of particular interest for engineering a microorganism to accumulate n-butanol or n-butanol derivative is the active phaA and phaB genes native to TIE-1, but these genes can also be introduced to enhance the enzymatic activity. Other gene of interest for engineering a microorganism to accumulate n-butanol are the phaJ, ter, and aghe2 genes. As shown herein, phaA and phaB are natively encoded on the Rhodopseudomonas palustris TIE-1 chromosome. Or the host can be engineered to carry more than one copy of the a non-natively expressed phaA or phaB gene.

In some embodiments, an enzyme encoding nucleotide sequence is cloned from its native source and inserted into a host microorganism. In some embodiments, a transformed host microorganism comprises a phaJ, ter, and adhE2, and optionally additional copies of phaA or phaB polynucleotide.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

The term transfection refers to the process of introducing nucleic acids into cells by non-viral methods.

The term transduction refers to the process whereby foreign DNA is introduced into another cell via a viral vector.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to transgenic microorganisms or reagents for genetically engineering a transgenic microorganism. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: N-Butanol Production by Rhodopseudomonas palustris TIE-1

The following example describes n-butanol production by Rhodopseudomonas palustris TIE-1 (TIE-1).

Abstract

Anthropogenic carbon dioxide (CO₂) release in the atmosphere from fossil fuel combustion has inspired scientists to study CO₂ to biofuel conversion. Oxygenic phototrophs such as cyanobacteria have been used to produce biofuels using CO₂. However, oxygen generation during oxygenic photosynthesis adversely affects biofuel production efficiency. To produce n-butanol (biofuel) from CO₂, herein is described an n-butanol biosynthesis pathway into an anoxygenic (non-oxygen evolving) photoautotroph, Rhodopseudomonas palustris TIE-1 (TIE-1). Using different carbon, nitrogen, and electron sources, n-butanol production was achieved in wild-type TIE-1 and mutants lacking electron-consuming (nitrogen-fixing) or acetyl-CoA-consuming (polyhydroxybutyrate and glycogen synthesis) pathways. The mutant lacking the nitrogen-fixing pathway produce the highest n-butanol. Coupled with novel hybrid bioelectrochemical platforms, this mutant produced n-butanol using CO₂, solar panel-generated electricity, and light with high electrical energy conversion efficiency. Overall, this approach showcases TIE-1 as an attractive microbial chassis for carbon-neutral n-butanol bioproduction using sustainable, renewable, and abundant resources.

### INTRODUCTION

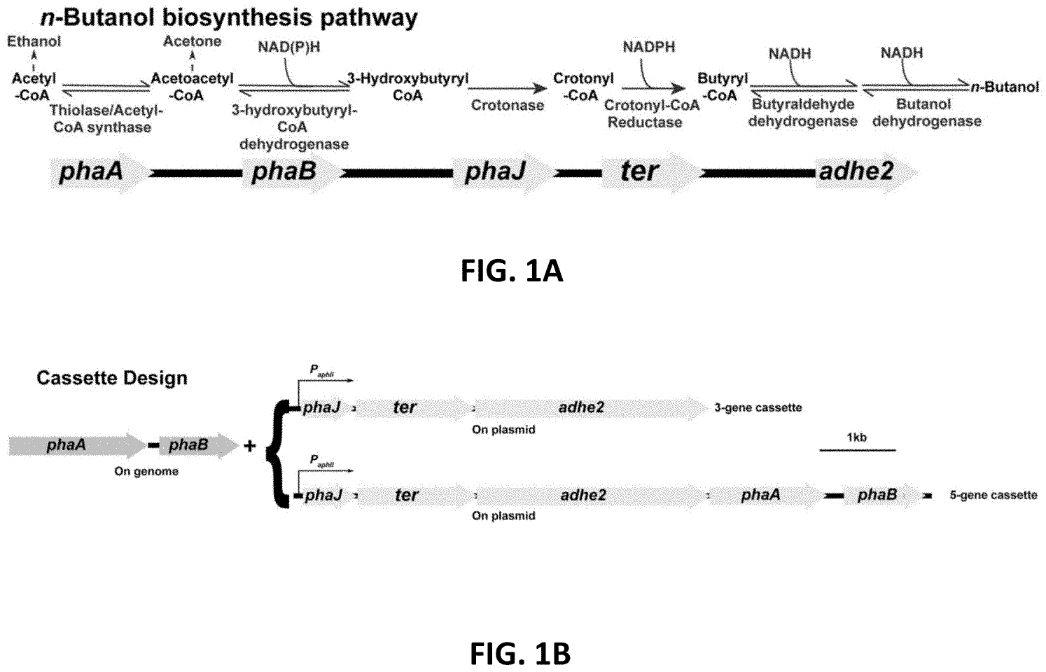

The rapid consumption of fossil fuels has increased carbon dioxide (CO₂) levels in the atmosphere raising concerns about global warming. This has spurred research initiatives aiming to develop carbon-neutral biofuels that, when burned, will not result in net CO₂ release. Among the various biofuels, n-butanol has received greater attention due to its higher energy content, lower volatility, and reduced hydrophilicity compared to ethanol. Currently, most n-butanol is synthesized via chemical processes. However, these processes use propylene or ethanol as feedstocks, making these methods carbon-positive. Another well-known strategy for n-butanol production is the acetone-butanol-ethanol (ABE) fermentation using Clostridium species. The n-butanol biosynthesis pathway (see e.g., FIG. 1A) from Clostridium acetobutylicum has been introduced into several organisms, such as Escherichia coli, Saccharomyces cerevisiae, Pseudomonas putida, and Bacillus subtilis for n-butanol production. However, most of these organisms are chemoheterotrophs. Thus, the n-butanol production using these microbes is also carbon-positive.

To date, only a handful of studies have produced n-butanol autotrophically using CO₂ as a carbon source. Using a microbial electrosynthesis approach, chemoautotroph Clostridium spp. produced 135 mg/L of n-butanol at an applied potential (Eₐₚₚₗ) of 0.8 V using CO₂ in 35 days. This prolonged period was required for acid accumulation for n-butanol production using Clostridium spp. Autotrophic n-butanol production was also demonstrated by an oxygenic photoautotroph Synechococcus elongatus PCC 7942 using water as an electron donor and sunlight as the energy source. Because the n-butanol was generated using solar energy, this product is called a solar fuel. With the n-butanol biosynthesis pathway, S. elongatus produced 2.2 mg/L n-butanol when incubated anaerobically under illumination. In contrast, aerobic incubation did not generate any n-butanol. Furthermore, a dark anaerobic incubation of dense cultures (where cells were not actively growing or evolving oxygen) produced 14 mg/L of n-butanol. These results suggest that oxygen (O₂) is detrimental to n-butanol production. The ability of cyanobacteria to produce n-butanol was later improved by several modifications such as (1) using cofactor as a driving force, (2) replacing the oxygen-sensitive enzyme involved in the n-butanol producing pathway, and (3) using intensive genetic engineering to optimize the pathway in a multi-level modular manner. These engineered cyanobacterial strains produced 29.9 mg/L, 404 mg/L, and 4.8 g/L of n-butanol. However, the low energy conversion efficiency (<3%) of natural photosynthesis makes the use of cyanobacteria not ideal for n-butanol production.

To enhance energy conversion efficiency for biofuel production, artificial photosynthesis, where photo-generated electrons were used to drive chemical reactions, was developed. However, due to catalyst limitations, hydrogen (H₂) was produced as the main product. Although H₂ can be used as a fuel, using such an explosive gas requires significant modifications to the current gasoline-based infrastructures. To avoid this, an H₂-consuming chemoautotrophic bacterium Ralstonia eutropha was used for producing carbon-based liquid fuels using a hybrid water-splitting biosynthetic system. In this system, H₂ and 02 were produced from water splitting (powered by electricity from a potentiostat) using a cobalt phosphorus catalyst with an applied electrical potential (Eₐₚₚₗ) of 2.0 V. The H2 was then fed to the engineered R. eutropha to synthesize C3-C5 alcohol or polyhydroxybutyrate (PHB) from CO₂. This hybrid biosynthetic system reached an electrical energy conversion efficiency (EECE) up to ˜20% using air (consists of 400 ppm CO₂) toward biomass. These values far exceeded the energy conversion efficiency of natural photosynthesis. Also, using genetically modified R. eutropha, the system reached an EECE of 16±2% towards C₄-C₅ alcohol using pure CO₂.

Also, coupling a solar panel with biosynthetic system resulted in an energy conversion efficiency of 6% towards biomass using pure CO₂. These studies indeed provided a platform for indirect solar fuel production from CO₂. However, this technology may not be an efficient and economical method for biofuel synthesis because, (1) it produces O₂, which is detrimental to many biofuel synthesis processes; (2) it uses H₂ as an electron donor, which due to its low solubility limits electron transfer efficiency and, finally; (3) this system requires electrical potentials higher than 1.23 V, making it an expensive method on the market. Therefore, it is critical to look for organisms that can overcome these limitations to advance carbon-neutral biofuel production.

One such organism is the anoxygenic photoautotroph Rhodopseudomonas palustris TIE-1 (TIE-1). TIE-1 can use various carbon sources, such as atmospheric CO₂ and organic acids that can be easily obtained from organic wastes. TIE-1 can also fix dinitrogen gas (N₂) and use a wide range of electron sources. These include H₂, which is a byproduct of many industries, ferrous iron [Fe(II)], which is a naturally abundant element. Most importantly, TIE-1 can also use electrons from poised electrodes (i.e., photoelectroautotrophy), that can be generated sustainably, for its photosynthetic growth. This wide electron donor selection enables TIE-1 to perform photosynthesis while avoiding O₂ generation, a harmful component for biofuel synthesis. TIE-1's ability to perform photoelectroautotrophy is advantageous for biofuel production, because the direct electron uptake by TIE-1 from a poised electrode avoids the need of an indirect electron donor such as H₂. TIE-1 has a low Eₐₚₚₗ (0.1 V) requirement, which lowers cost and electrochemical O₂ generation. TIE-1's Eₐₚₚₗ requirement is ˜90% lower than that needed for water-splitting, which allows the use of low-cost solar panels to build novel biohybrid systems for solar fuel synthesis. Overall, TIE-1 is a superlative biocatalyst that allows us to use extant CO₂, N₂, solar energy, and electrons generated by renewable electricity for bioproduction. This process enables excess electricity to be stored as a usable fuel or product for later use.

In a previous study, R. palustris CGA009 (CGA009), a strain closely related to TIE-1, was engineered to produce n-butanol from n-butyrate. In that study, the gene encoding the alcohol/aldehyde dehydrogenases (AdhE2) from R. palustris Bisb was codon-optimized and introduced into CGA00933. When cultured in the absence of CO₂, the modified CGA009 was forced to reduce n-butyrate into n-butanol to maintain the redox balance. Although this study used a phototroph for n-butanol production, the use of an organic substrate makes this approach carbon-positive.

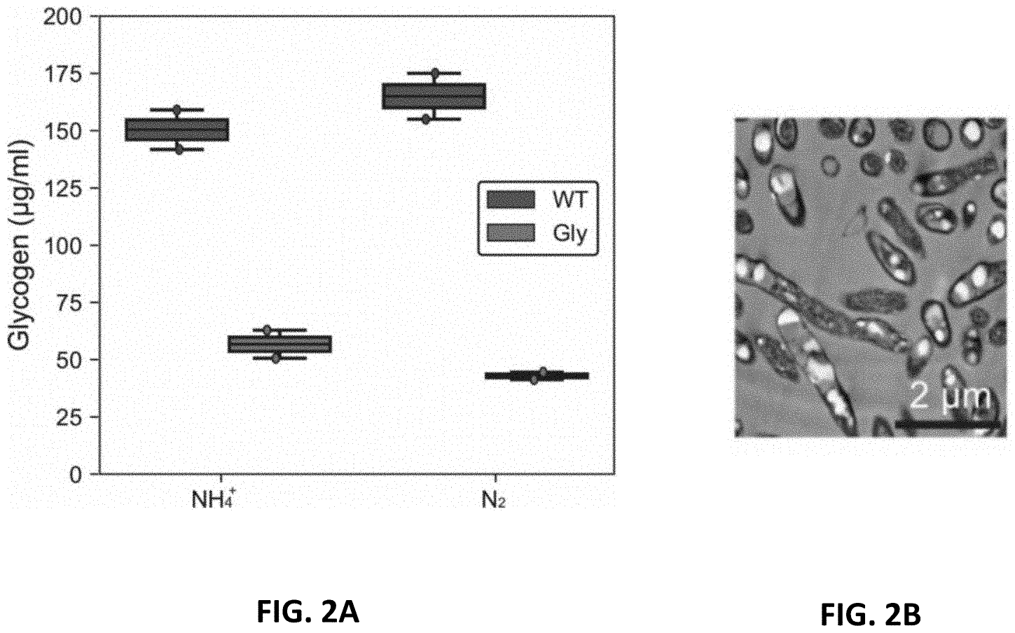

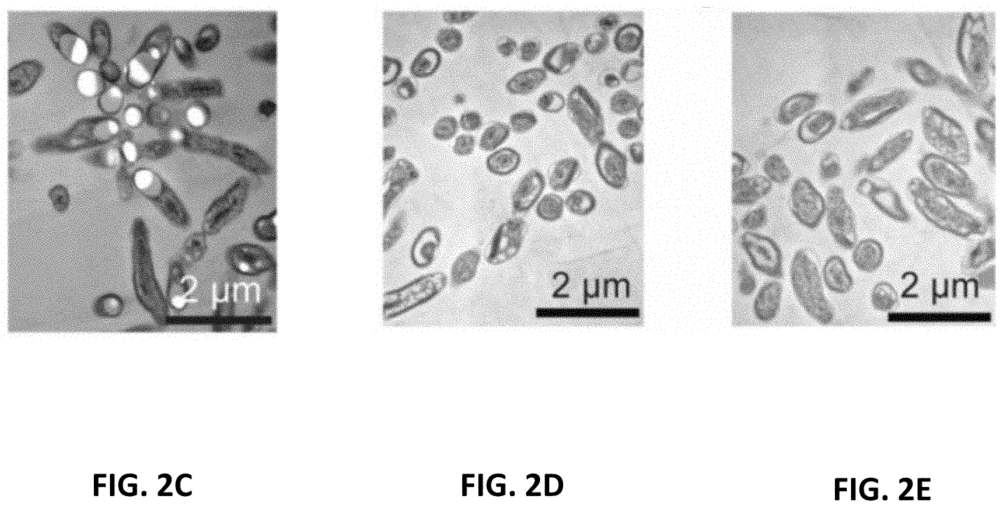

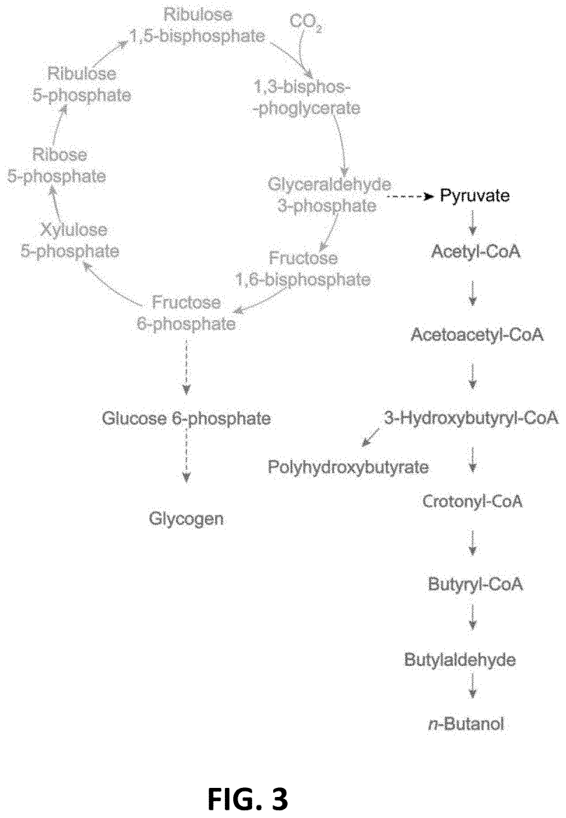

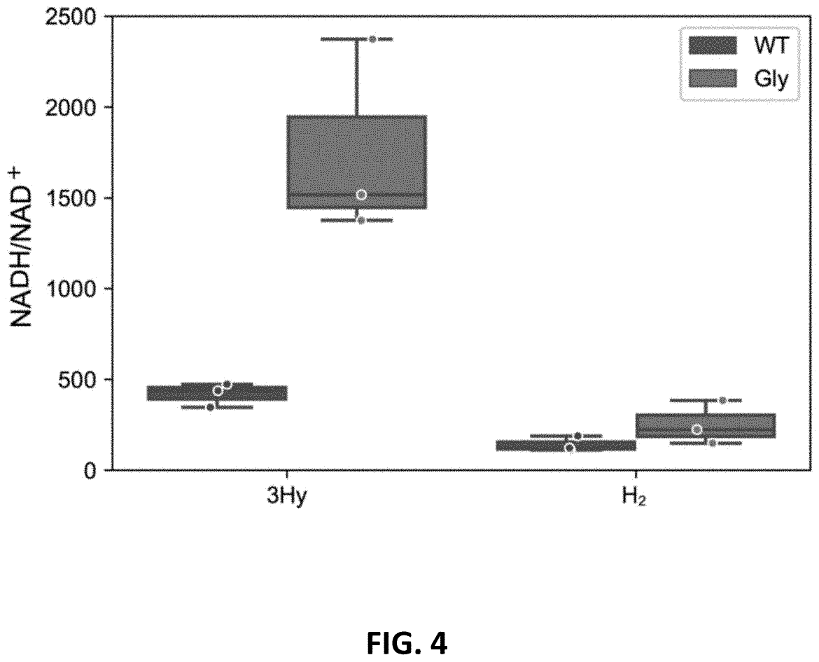

To produce n-butanol in a sustainable and carbon-neutral manner, described herein is the introduction of an efficient, codon-optimized n-butanol biosynthesis pathway into TIE-1. This pathway was assembled using irreversible and efficient enzymes and produced 4.6 g/L n-butanol in E. coli. The pathway contains five genes (phaA, phaB, phaJ, ter, adhE2). Because TIE-1 possesses homologs for the first two genes (phaA and phaB), two different cassettes were designed (see e.g., FIG. 1B), containing either the whole (5-gene cassette) or a partial n-butanol biosynthesis pathway (3-gene cassette). As shown in FIG. 1A, carbon (acetyl-CoA) and reducing equivalents (NADH) are two major substrates for n-butanol biosynthesis. Previous studies in cyanobacteria have shown that a PHB synthase deletion mutant produces more butanol, and a glycogen synthase deletion mutant showed higher carbon conversion efficiency (CCE) towards iso-butanol. Therefore, TIE-1 knockout mutants lacking hydroxybutyrate polymerase (phaC1, Rpal_2780 and phaC2, Rpal_4722) or glycogen synthase (glgA, Rpal_0386) were constructed. As expected, these mutants accumulated significantly less PHB and glycogen compared to wild-type (WT) (see e.g., FIG. 2A-FIG. 2E). The detailed carbon flow between n-butanol synthesis, PHB synthesis, and glycogen synthesis is shown in FIG. 3. Previous studies suggested that nitrogenase deletion mutants possess a more reduced intracellular environment in Rhodobacter capsulatus and CGA00937-39. As this may be true in TIE-1 as well, a double mutant (Nif mutant) was created by deleting nifA1 (Rpal_5113) and nifA2 (Rpal_1624), which are the regulators that potentially activate their cognate clusters of nitrogenase genes, namely the putative Molybdenum-dependent nitrogenase and the putative Iron-dependent nitrogenase. The increased NADH/NAD+ ratio observed in the Nif mutant compared to WT, when using 3-hydroxybutyrate as electron donor (FIG. 4) indicated a more reduced intracellular environment. In addition, the inability of the Nif mutants to grow under nitrogen-fixing conditions (TABLE 1) further indicated the loss of nitrogen-fixing capability of the Nif mutant.

TABLE 1
Doubling time of ΔnifA1ΔnifA2 using different carbon and
nitrogen source.

| Incubation Conditions | Doubling time (hr) | Doubling time (hr) |
|---|---|---|
| 3-Hydroxybutyrate + | 9.4 ± 2.5 | 11.2 ± 1.8 |
| NH₄⁺ | | |
| 3-Hydroxybutyrate + N₂ | No growth | 13.4 ± 1.2 |
| H₂ + NH₄ | 218.5 ± 65.0 | 143.9 ± 63.7 |
| H₂ + N₂ | No growth | 100.4 ± 3.2 |

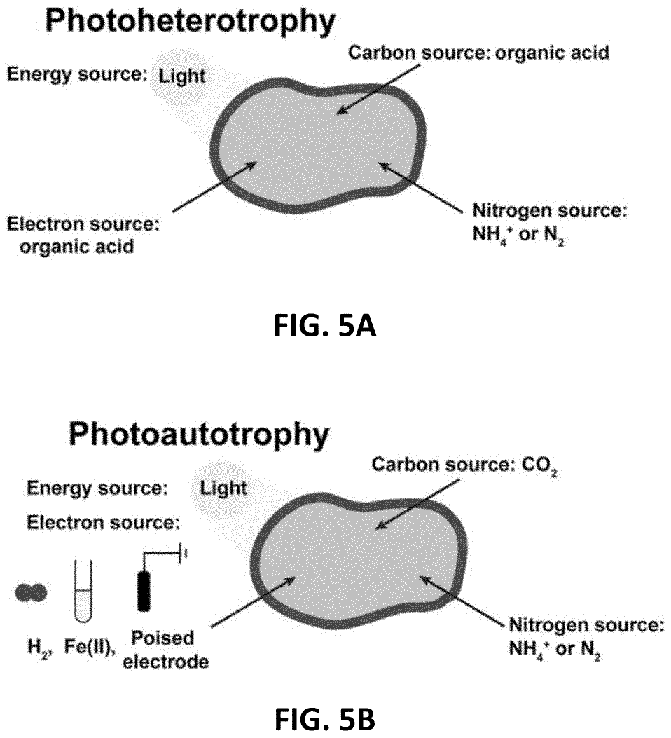

After introducing the 3-gene cassette/5-gene cassette into the TIE-1 WT and mutant strains, n-butanol production was tested under both photoheterotrophic (see e.g., FIG. 5A) and photoautotrophic (see e.g., FIG. 5B) conditions. Under photoelectroautotrophy, a novel hybrid bioelectrochemical cell (BEC) platform powered by electricity supplied from either potentiostat or a solar panel was used.

The results show that the anoxygenic phototroph TIE-1 can produce n-butanol sustainably using organic acids or CO₂ as a carbon source, light as an energy source, and H₂, Fe(II), or electrons from renewably generated electricity as an electron source. Described herein is the first attempt for biofuel production using a solar panel-powered microbial electrosynthesis platform, where CO₂ is directly converted to liquid fuel. Overall, these results show that TIE-1 may be an attractive future microbial chassis for producing carbon-neutral biofuels via synthetic biology and metabolic engineering.

Results

Deleting an Electron-Consuming Pathway or Low Cell Growth Enhances n-Butanol Production.

N-butanol production by WT with 3-gene cassette (WT-3), WT with 5-gene cassette (WT-5), and TIE-1 mutants with either 3-gene (Nif-3, Gly-3, Phb-3) or 5-gene cassette (Nif-5, Gly-5), under various photoheterotrophic and photoautotrophic conditions (substrate combinations, incubation time and final optical density listed in TABLE 2-TABLE 4) to identify the most productive strains and conditions.

TABLE 2
Incubation combinations used in this study (except for
photoelectroautotrophy).

| Incubation | | | Electron | |
|---|---|---|---|---|
| condition | | Nitrogen Sources | Sources | Carbon Sources |
| 1 | Ac—NH₄⁺ | Ammonium | Acetate (Ac) | Acetate |
| | | (NH₄⁺) | | |
| 2 | Ac—N₂ | Dinitrogen gas | | |
| | | (N₂) | | |
| 3 | 3Hy—NH₄⁺ | Ammonium | 3-hydroxybutyrate | 3-hydroxybutyrate |
| 4 | 3Hy—N₂ | Dinitrogen gas | (3Hy) | |
| 5 | H₂—NH₄⁺ | Ammonium | Hydrogen (H₂) | Carbon Dioxide |
| 6 | H₂—N₂ | Dinitrogen gas | | (CO₂) |
| 7 | Fe(II)—NH₄⁺ | Ammonium | Ferrous Iron | |
| 8 | Fe(II)—N₂ | Dinitrogen gas | (Fe(II)) | |

TABLE 3
Incubation time of all cultures.

| | Ac—NH₄⁺ | Ac—N₂ | 3Hy-NH₄⁺ | 3Hy-N₂ | H₂—NH₄⁺ | H₂—N₂ | Fe—NH₄⁺ | Fe—N₂ | EU |
|---|---|---|---|---|---|---|---|---|---|
| WT + 3 | 10 | | | | 13 | | | | n.a. |
| WT + 5 | days | | | | days | | | | |
| Nif + 3 | 13 | 16 | 13 | 16 | 16 | 19 | 16 | 19 | |
| Nif + 5 | days | days | days | days | days | days | days | days | 10 |
| | | | | | | | | | days |

| | | | |
|---|---|---|---|
| Gly + 3 | 10 | 13 | n.a. |
| Gly + 5 | days | days | |
| Phb + 3 | | | |

Ac: acetate;
3Hy: 3-hydroxybutyrate;
H2: hydrogen;
Fe(II): ferrous iron;
CO₂: carbon dioxide;
NH₄⁺: ammonium;
N₂: dinitrogen gas
EU: photoelectroautotrophy
WT-3: WT with 3-gene cassette;
WT-5: WT with 5-gene cassette;
Nif-3: ΔnifA1ΔnifA2 with 3-gene cassette;
Nif-5: ΔnifA1ΔnifA2 with 5-gene cassette;
Gly-3: ΔglgA with 3-gene cassette;
Gly-5: ΔglgA with 5-gene cassette;
Phb-3: ΔphaC1ΔphaC2 with 3-gene cassette;
n.d.: non-detectable;
n.a.: not applicable

TABLE 4
Final optical density (OD660) of all the construct under n-butanol producing conditions.

| | Ac—NH₄⁺ | | Ac—N₂ | | 3Hy-NH₄⁺ | | 3Hy-N₂ | | H₂—NH4⁺ | | H₂—N₂ | | Fe—NH₄⁺ | | Fe—N₂ | |
|---|---|---|---|---|---|---|---|---|---|---|---|---|---|---|---|---|
| | ave | std | ave | std | ave | std | ave | std | ave | std | ave | std | ave | std | ave | std |
| WT + 3 | 4.13 | 0.85 | 3.98 | 0.25 | 2.71 | 0.32 | 2.62 | 0.52 | 1.23 | 0.09 | 0.99 | 0.16 | 1.22 | 0.11 | 1.14 | 0.28 |
| WT + 5 | 5.14 | 0.49 | 1.52 | 0.32 | 3.61 | 0.19 | 1.44 | 0.08 | 1.67 | 0.16 | 1.53 | 0.02 | 1.89 | 0.05 | 1.73 | 0.10 |
| Nif + 3 | 4.33 | 0.21 | 2.20 | 0.04 | 2.46 | 0.21 | 0.86 | 0.03 | 1.72 | 0.15 | 1.78 | 0.10 | 1.17 | 0.31 | 1.15 | 0.07 |
| Nif + 5 | 3.30 | 0.44 | 1.12 | 0.02 | 2.92 | 0.17 | 1.05 | 0.03 | 1.85 | 0.05 | 1.20 | 0.19 | 1.73 | 0.06 | 0.74 | 0.05 |
| Gly + 3 | 7.46 | 1.72 | 3.96 | 0.87 | 7.24 | 0.97 | 4.04 | 0.72 | 1.04 | 0.32 | 0.78 | 0.11 | 0.83 | 0.09 | 0.77 | 0.05 |
| Gly + 5 | 4.86 | 0.60 | 2.47 | 0.31 | 3.26 | 0.18 | 1.86 | 0.08 | 1.41 | 0.07 | 1.54 | 0.27 | 1.56 | 0.11 | 1.44 | 0.08 |
| Phb + 3 | 3.93 | 0.45 | 1.99 | 0.68 | 5.78 | 0.37 | 1.68 | 0.45 | 2.02 | 0.09 | 2.00 | 0.45 | 1.40 | 0.06 | 2.05 | 0.15 |

ave: average,
std: standard deviation
Ac: acetate;
3Hy: 3-hydroxybutyrate;
H₂: hydrogen;
Fe(II): ferrous iron;
CO₂: carbon dioxide;
NH₄⁺: ammonium;
N₂: dinitrogen gas
EU: photoelectroautotrophy
WT-3: WT with 3-gene cassette;
WT-5: WT with 5-gene cassette;
Nif-3: ΔnifA1ΔnifA2 with 3-gene cassette;
Nif-5: ΔnifA1ΔnifA2 with 5-gene cassette;
Gly-3: ΔglgA with 3-gene cassette;
Gly-5: ΔglgA with 5-gene cassette;
Phb-3: ΔphaC1ΔphaC2 with 3-gene cassette;

Because the adaptation phase performed for slow growth conditions such as nitrogen-fixing conditions resulted in plasmid loss, all the experiments were performed by incubating dense aerobically pre-grown cells (OD660=1). These cells were washed and used as inoculum for the anaerobic conditions using various electron and carbon sources. The Nif mutants incubated under nitrogen-fixing conditions are non-growing. For all the other mutants and incubation conditions, the final optical density is listed in TABLE 4.

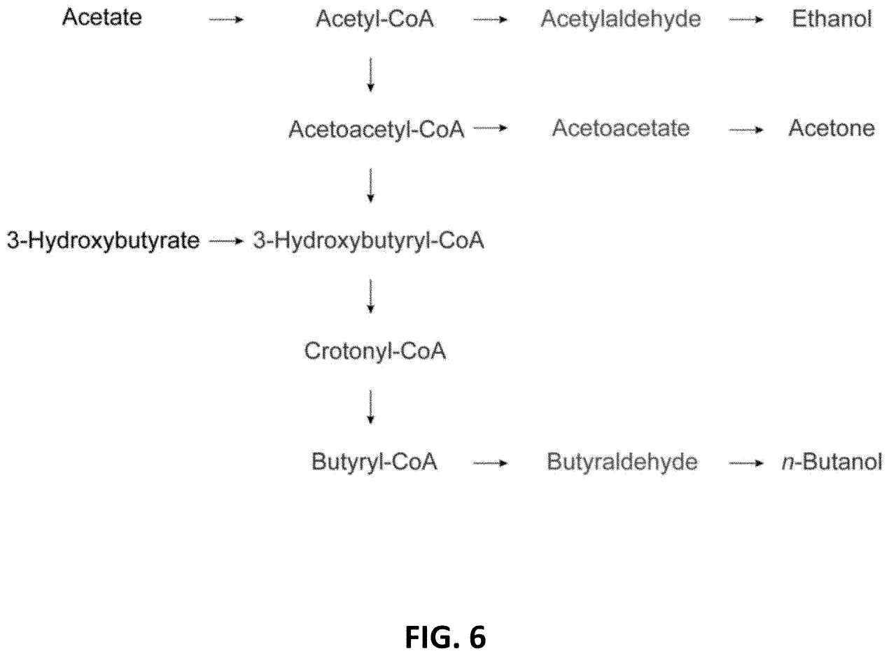

For photoheterotrophic conditions, acetate (Ac) or 3-hydroxybutyrate (3Hy) was chosen as carbon and electron sources because both substrates enter the n-butanol biosynthesis pathway directly as their CoA derivatives (see e.g., FIG. 6). For photoautotrophic conditions, either H₂ or Fe(II) was used as an electron donor. CO₂ was supplied in all conditions to maintain the pH of the medium and for redox balance in the cell. N₂ or ammonia (NH₄⁺) was provided as the nitrogen source.

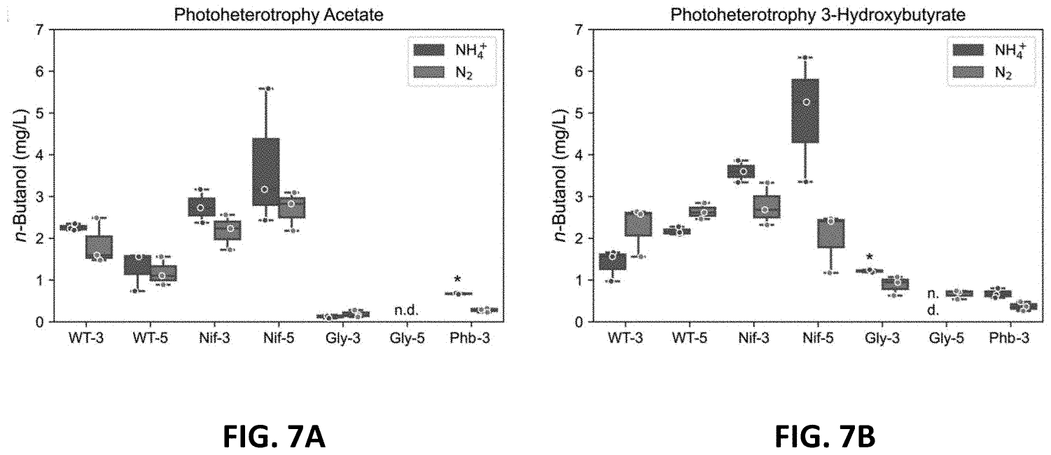

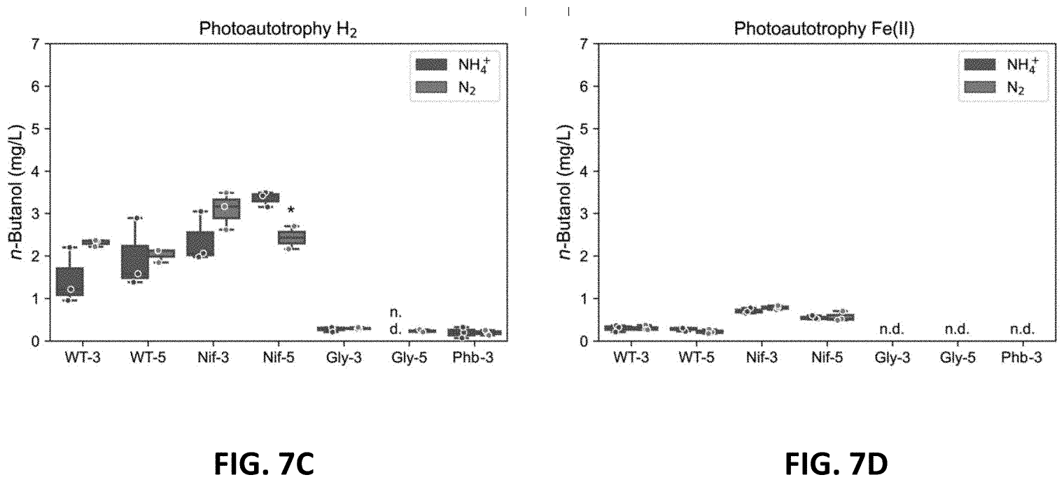

Depending on the carbon and electron source, the same construct produced variable amounts of n-butanol. n-Butanol production was the highest in the presence of 3Hy, followed by H₂, Ac, and Fe(II) (see e.g., FIG. 7A-FIG. 7D). Nif-5 was the most efficient n-butanol producer with the highest production of 4.98±0.87 mg/L under the photoheterotrophic conditions with NH₄⁺ (see e.g., FIG. 7B). The same construct, however, produced ˜10-fold lower n-butanol when incubated with Fe(II) (0.55±0.03 mg/L) (see e.g., FIG. 7D).

Compared to WT-3/WT-5, Nif-3/Nif-5 produced similar or more n-butanol depending on the substrates, whereas Gly-3/Gly-5 and Phb-3 produced less n-butanol regardless of the substrate type (see e.g., FIG. 7A-FIG. 7D). The presence of NH₄⁺ in the media has been reported to repress the nitrogenase genes. Therefore, in its presence, WT-3/WT-5 and Nif-3/Nif-5 may produce similar amounts of n-butanol. Surprisingly, in most cases, Nif-3/Nif-5 produced a higher amount of n-butanol than the WT-3/WT-5, even in the presence of NH₄⁺ (see e.g., FIG. 7A-FIG. 7D). Overall, deleting an electron-consuming pathway (Nif) was beneficial, whereas deleting an acetyl-CoA-consuming pathway (Gly and Phb) was detrimental to n-butanol production. However, the increased n-butanol production in Nif-3/Nif-5 may also be a result of low cell growth (see e.g., TABLE 4).

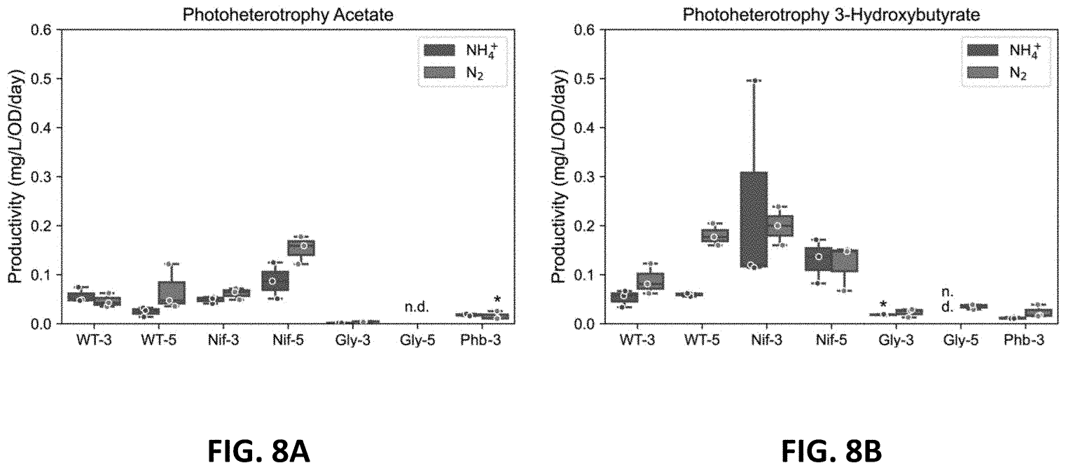

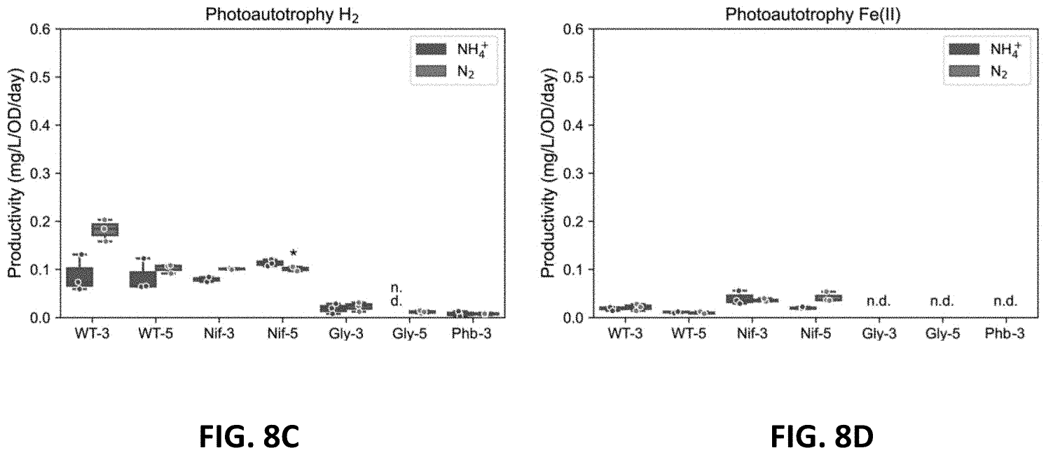

Because the multiple electron donor choices resulted in different incubation times, and final optical density, the productivity (n-butanol mg/L/day/OD660) by each strain under each incubation condition was calculated to make a meaningful comparison. For most strain constructs, incubation with 3Hy showed the highest productivity, followed by H₂, Ac, and Fe(II) (see e.g., FIG. 8A-FIG. 8D). Among all the strain constructs, Nif-3/Nif-5 showed the highest productivity under most incubation conditions.

No n-butanol was detected from WT with an empty vector using 3Hy as a carbon source and NH₄⁺ as a nitrogen source. To ensure the n-butanol production is not toxic to TIE-17, a toxicity assay was performed. The lowest inhibitory concentration of n-butanol for TIE-1 is 4050 mg/L (see e.g., TABLE 5), which is about 1000-fold higher than the highest n-butanol produced in the experiment (4.98±0.87 mg/L).

TABLE 5
Doubling time of WT with pAB423 in media supplied with different
amounts of n-butanol.

| | Concentration of n-Butanol (mg/L) | | | | |
|---|---|---|---|---|---|
| | 0 | 2025 | 4050 | 8100 | 16200 |
| Doubling time | 15.00 | 20.091 | No growth | No growth | No growth |
| (hr) | | | | | |

Hence, the n-butanol produced during this study does not limit the growth of TIE-1.

Deleting Acetyl-CoA-Consuming Pathways Diverts Carbon to Acetone Production.

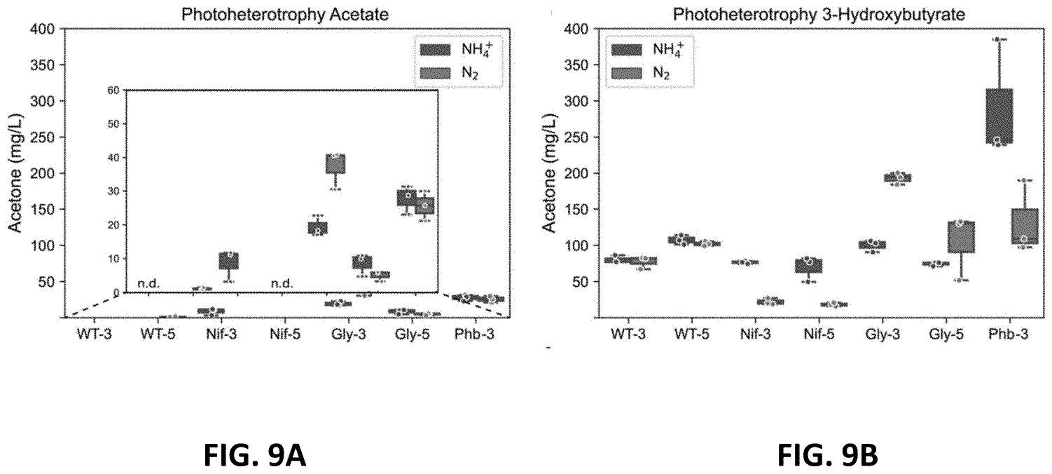

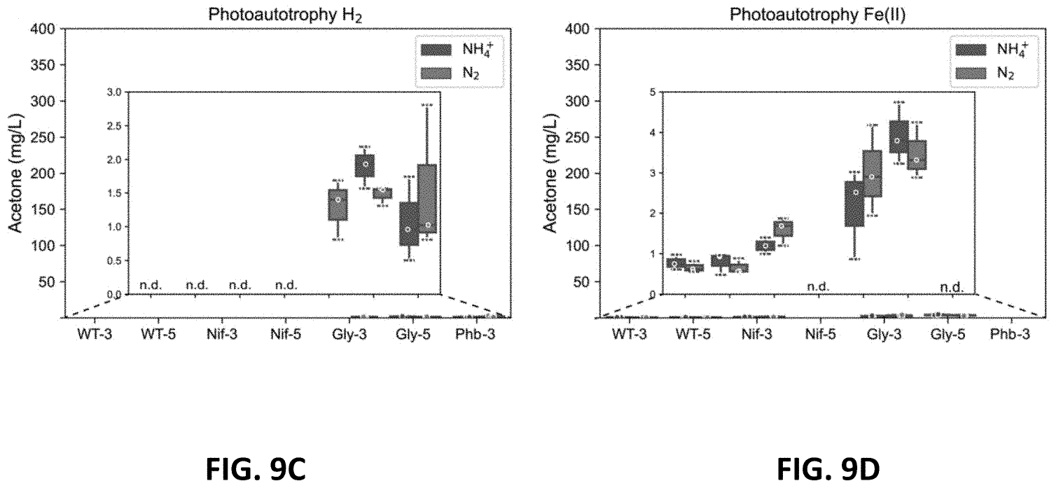

Acetone is a major byproduct of n-butanol biosynthesis which is produced by the accumulation of acetoacetyl-CoA, an intermediate product in n-butanol biosynthesis (see e.g., FIG. 1A and FIG. 6). The highest acetone production was observed by Phb-3 (0.00 to 290.01±47.51 mg/L) followed by Gly-3/Gly-5 (1.47±0.08 mg/L to 192.84±4.82 mg/L), WT-3/WT-5 (0.00 mg/L to 107.39±3.74 mg/L), and Nif-3/Nif-5 (0.00 mg/L to 76.44±1.12 mg/L) (see e.g., FIG. 9A-FIG. 9D). This acetone production trend is in the reverse trend of n-butanol production, i.e., Nif-3/Nif-5 produced the highest, and the Phb-3 produced the lowest amount of n-butanol (see e.g., FIG. 7A-FIG. 7D). These results indicate that acetone biosynthesis likely competes for acetyl-CoA with n-butanol biosynthesis. Using either Phb or Gly, acetyl-CoA that would have otherwise been directed toward PHB or glycogen synthesis was diverted to acetone biosynthesis.

Compared to using Ac as a substrate, which produced 0.00 to 37.29±3.40 mg/L of acetone, all constructs produced ˜10-100-fold more acetone when supplied with 3Hy (18.15±1.41 to 290.10±38.80 mg/L (see e.g., FIG. 9A-FIG. 9B). However, when the same strain was used, the acetone production under photoautotrophic conditions was lowered by ˜25-125-fold compared to photoheterotrophic conditions with only 0.00 to 3.92±0.44 mg/L (see e.g., FIG. 9C-FIG. 9D). These results indicate that under photoheterotrophic conditions, particularly with 3Hy, TIE-1 accumulates more acetyl-CoA, which is eventually converted into acetone. The high acetone production suggests that acetyl-CoA is not limiting n-butanol production. The acetone toxicity in TIE-1 was also tested, and the amount of acetone produced does not limit TIE-1's growth (see e.g., FIG. 9A-FIG. 9D and TABLE 6).

TABLE 6
WT with pAB423 in media supplied with deferent amounts of
acetone.

| | Concentration of Acetone (mg/L) | | | | |
|---|---|---|---|---|---|
| | 0 | 1960 | 3920 | 7840 | 15680 |
| Doubling time | 7.30 | 6.74 | 5.82 | 5.56 | 4.88 |
| (hr) | | | | | |

More Reducing Equivalents or Low Cell Growth Enhances Carbon Conversion Efficiency (CCE) to n-Butanol.

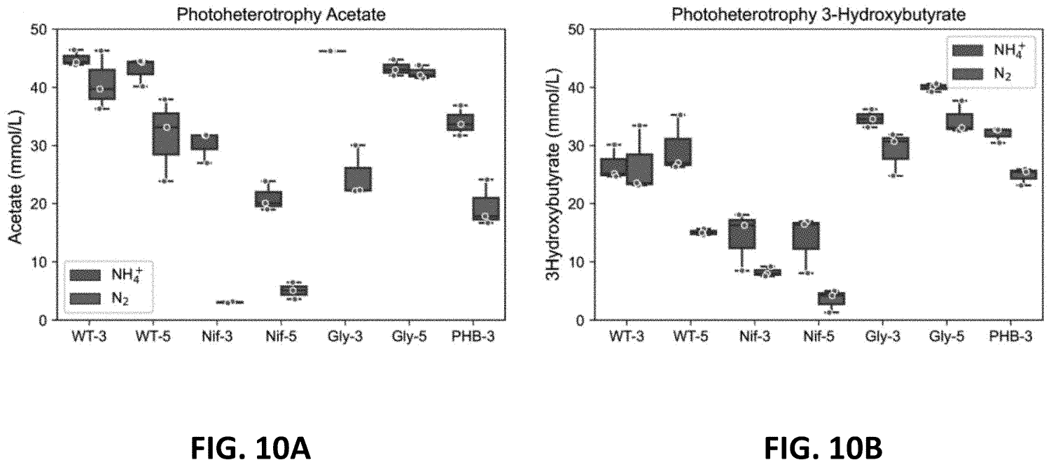

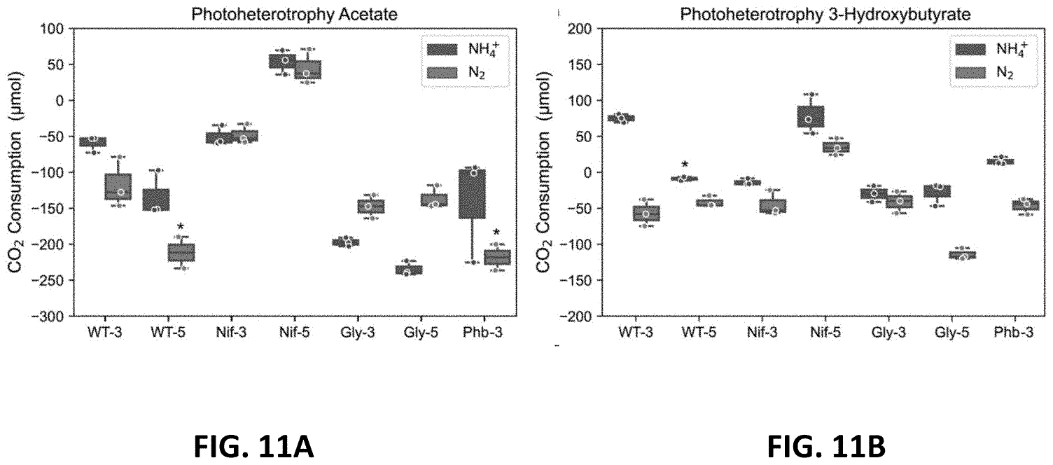

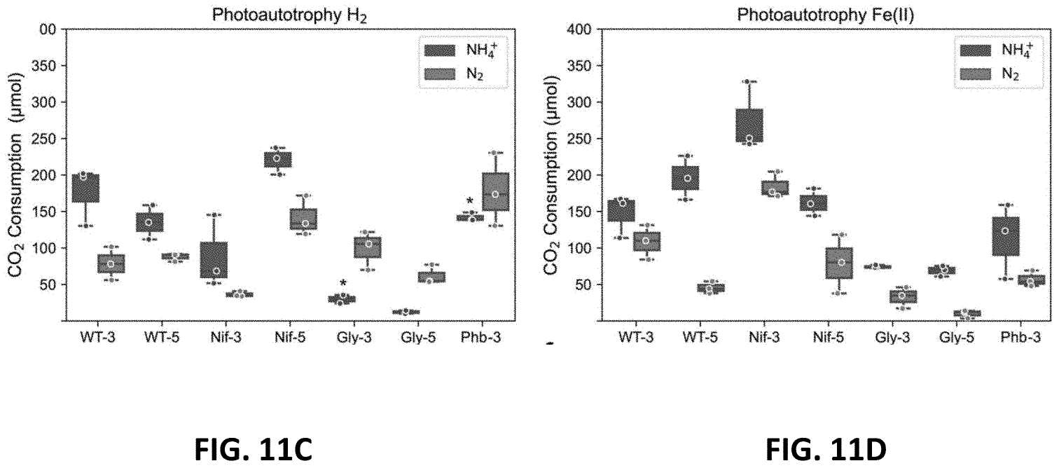

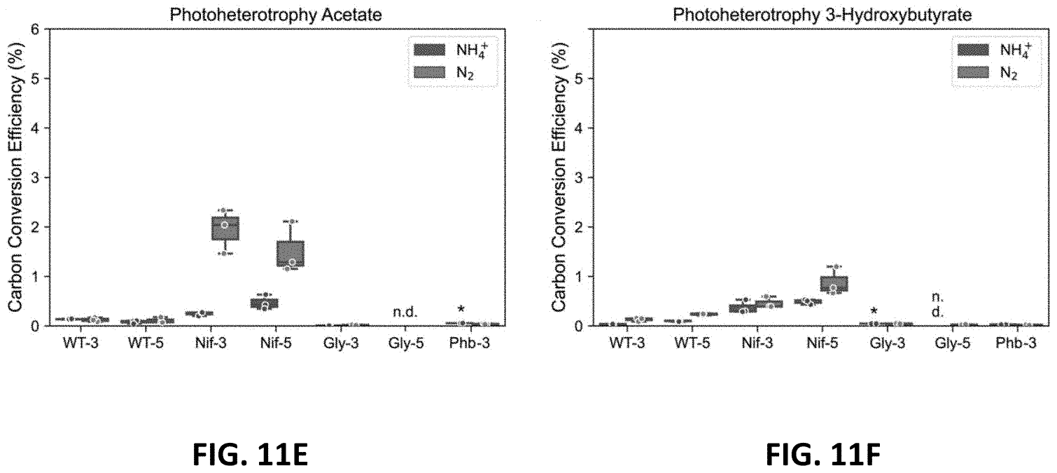

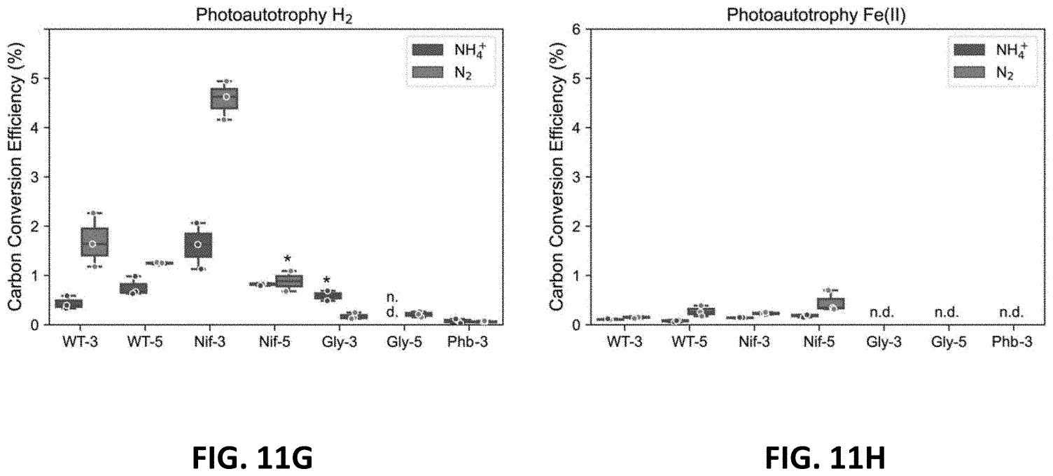

To further identify the most efficient strain and substrate for n-butanol production with respect to carbon, carbon consumption (see e.g., FIG. 10A-FIG. 10B and FIG. 11A-FIG. 11D) and CCE (see e.g., FIG. 11E-FIG. 11H) towards n-butanol was determined for each construct under all conditions.

Carbon consumption. It was recently shown that TIE-1 can fix CO₂ during photoheterotrophic growth. Therefore, CO₂ consumption and generation by all constructs was also calculated. Under photoheterotrophy, all TIE-1 constructs consumed more (or generated less, represented by smaller negative value) CO₂ with 3Hy (up to −114.23±4.52 to 78.67±15.86 μmol) than Ac (up to −243.67±5.79 to 53.79±9.77 μmol) (see e.g., FIG. 11A-FIG. 11B). With either 3Hy or Ac, Nif-3/Nif-5 consumed more CO₂ (or generated less) (CO₂ generation: −50.53±8.01 to 78.67±15.86 μmol) (see e.g., FIG. 11A-FIG. 11B). These results are consistent with a previous study where the use of a more reduced substrate (such as 3Hy) resulted in more carbon consumption than the use of a more oxidized substrate (such as acetate) for redox balance.

Similarly, under photoautotrophic conditions, Nif-3/Nif-5 consumed the highest amount of CO₂ (36.41±2.17 to 273.76±27.25 μmol), except for Nif-3 incubated with H₂ and NH₄⁺ (see e.g., FIG. 11C-FIG. 11D). This observation is likely due to the higher CO₂ fixation required to achieve redox balance in the absence of N₂-fixation. Gly-3/Gly-5 consumed the lowest amount of CO₂, ranging from −234.67±5.79 to 99.04±15.32 μmol (see e.g., FIG. 11C-FIG. 11D). A previous study using glycogen mutants has been reported to fix less CO₂ compared to WT in cyanobacteria. This observation corroborates with the finding that Gly-3/Gly-5 produces low n-butanol under photoautotrophic conditions (see e.g., FIG. 7C-FIG. 7D).

CCE. CCE is defined as moles of carbon in n-butanol divided by moles of carbon from substrates. (see method section for detailed calculations). Similar to the trend for n-butanol production (see e.g., FIG. 7A-FIG. 7D), Nif-3/Nif-5 showed the highest CCE towards n-butanol (0.12±0.03 to 4.58±0.23%), followed by WT-3/WT-5 (0.03±0.01 to 1.70±0.32%), Gly-3/Gly-5 (0.00 to 0.59±0.10%), and Phb-3 (0.00 to 0.16±0.04%) (see e.g., FIG. 11E-FIG. 11H). These results suggest that excess reducing equivalents enhanced n-butanol production and facilitated CCE to n-butanol. Similar to the results from n-butanol production, this may also be due to low cell growth. In contrast, lack of the PHB or glycogen biosynthesis decreased overall CCE to n-butanol. All strains had the highest CCE when incubated with H₂ (0.00 to 4.58±0.23%), except for Phb (see e.g., FIG. 11G), which was unable to produce n-butanol using any substrate (see e.g., FIG. 7C). This high CCE in the presence of H₂ (see e.g., FIG. 11G) may be due to low acetone production (see e.g., FIG. 9C) and the lower cell growth compared to the other conditions. Higher CCE towards n-butanol (1- to 7-fold) was observed when Nif-3/Nif-5 was supplied with N₂ compared to NH₄⁺. For example, in the presence of NH₄⁺, Nif-3/Nif-5 showed CCE of 0.14±0.01 to 1.61±0.27%, which increased to 0.23±0.01 to 4.58±0.23% when N₂ was provided (see e.g., FIG. 11E-FIG. 11H). This result also indicated that low cell growth might lead to higher CCE towards n-butanol biosynthesis by TIE-1.

More Reducing Equivalents and Low Cell Growth Enhances Electron Conversion Efficiency to n-Butanol.

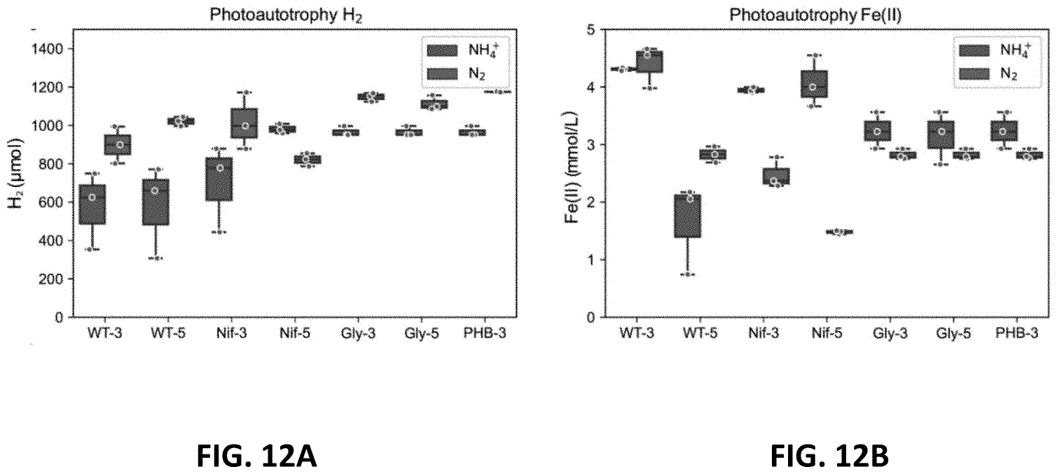

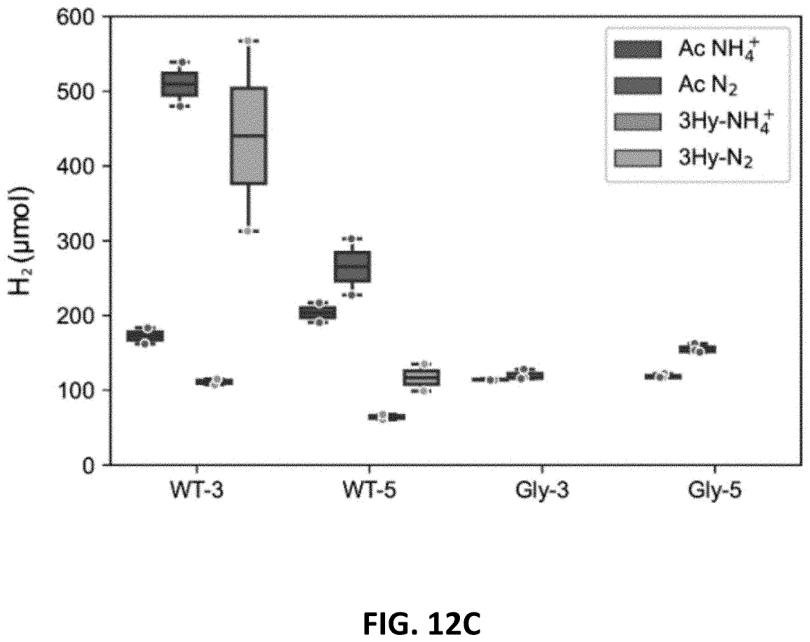

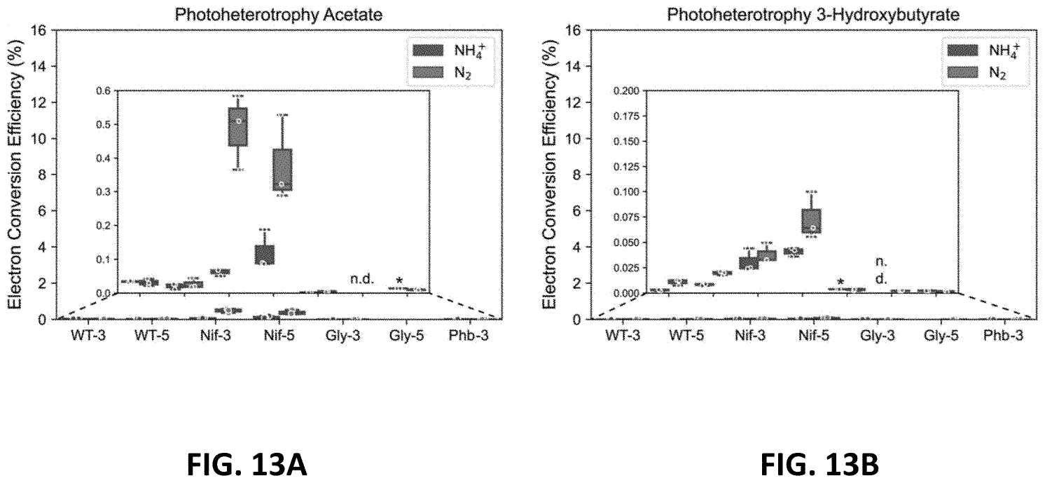

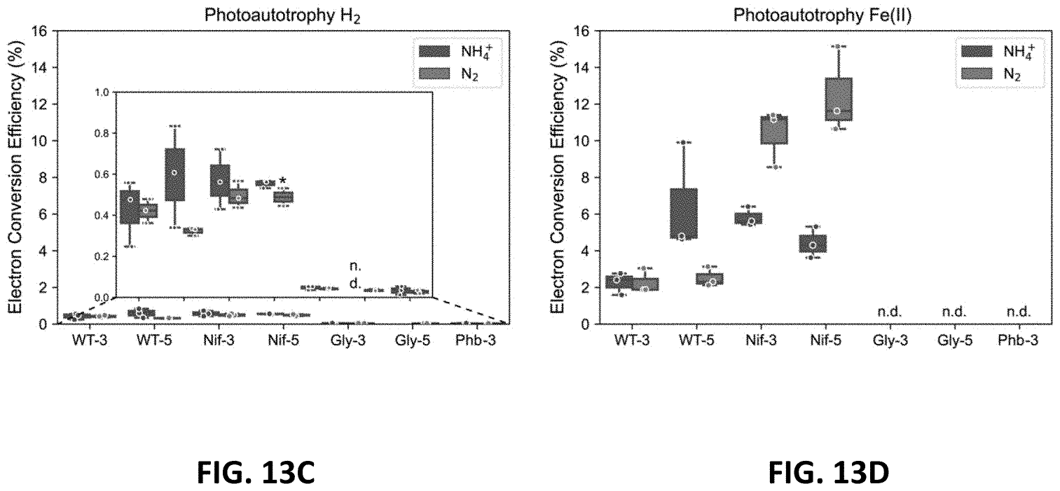

To further identify the most productive strain and substrate toward n-butanol production with respect to electron availability, each construct's electron conversion efficiency (ECE) towards n-butanol (electron donor consumption data shown in FIG. 10A-FIG. 10B and FIG. 12A-FIG. 12C) was calculated. ECE is defined as the moles of the electron in n-butanol divided by the moles of the electron from substrates (see method section for detailed calculations). Photoautotrophic conditions reached higher ECE towards n-butanol than photoheterotrophic conditions. With an ECE of 0.00 to 12.47±1.37%, Fe(II) was the most favorable electron donor followed by H₂ (0.00 to 0.59±0.14%), Ac (0.00 to 0.49±0.06%), and 3Hy (0.00 to 0.07±0.01%) (see e.g., FIG. 13A-FIG. 13D). The highest ECE towards bioplastic in the presence of Fe(II) has also been observed previously.

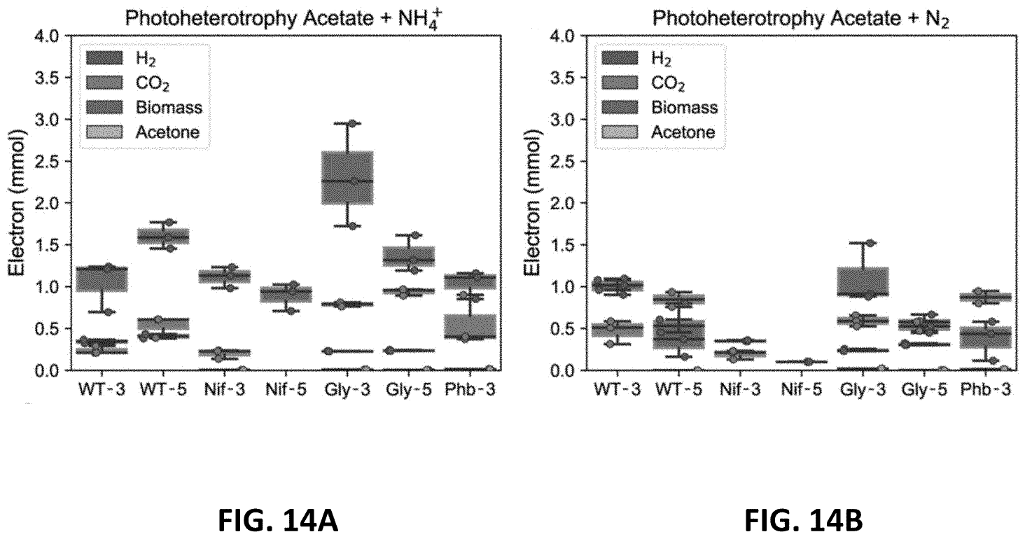

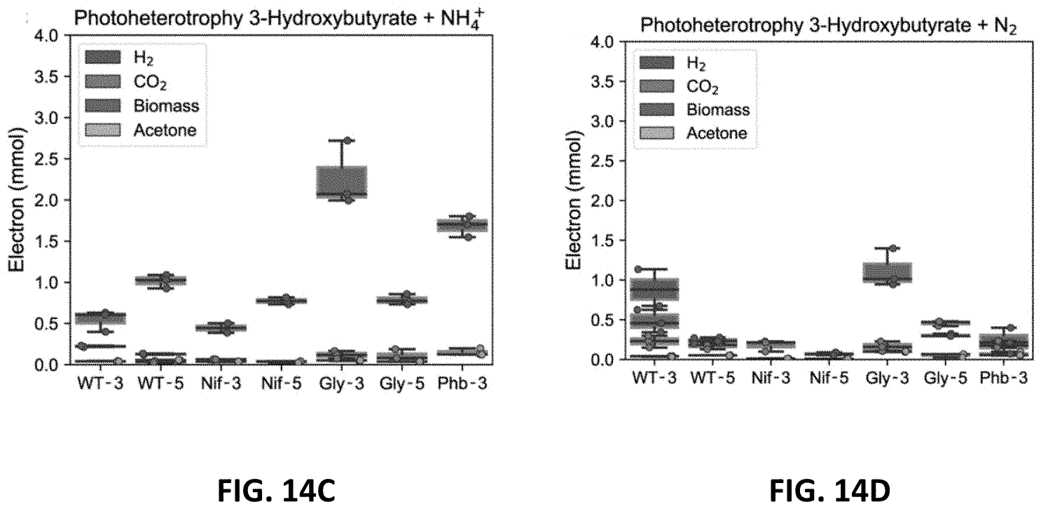

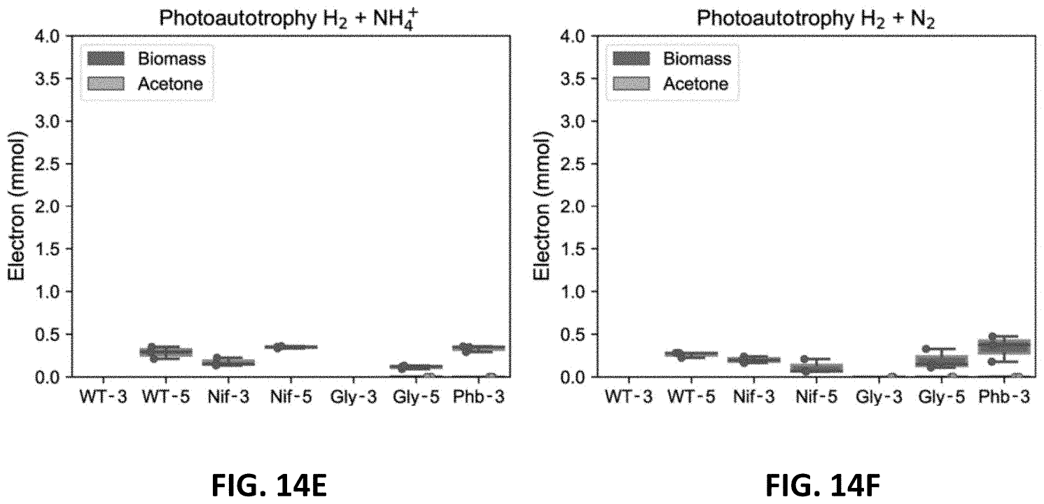

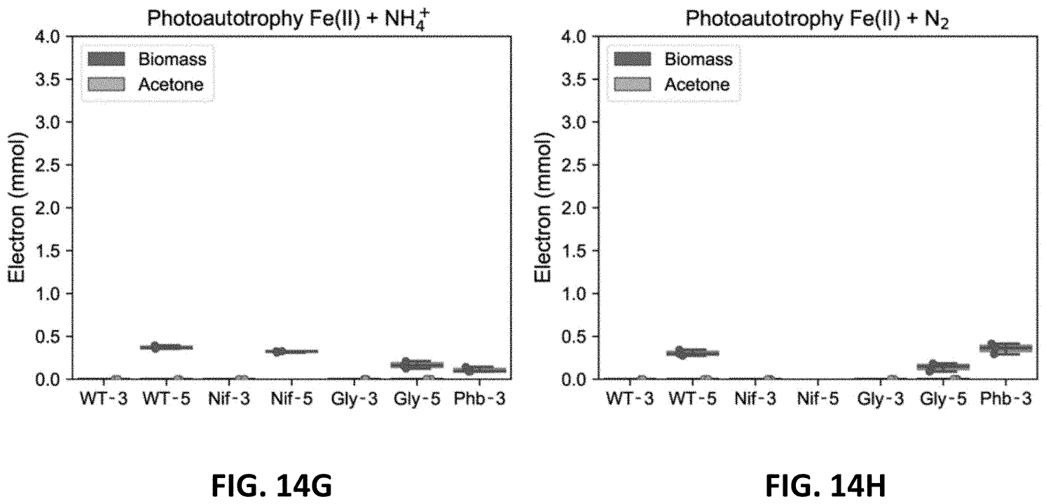

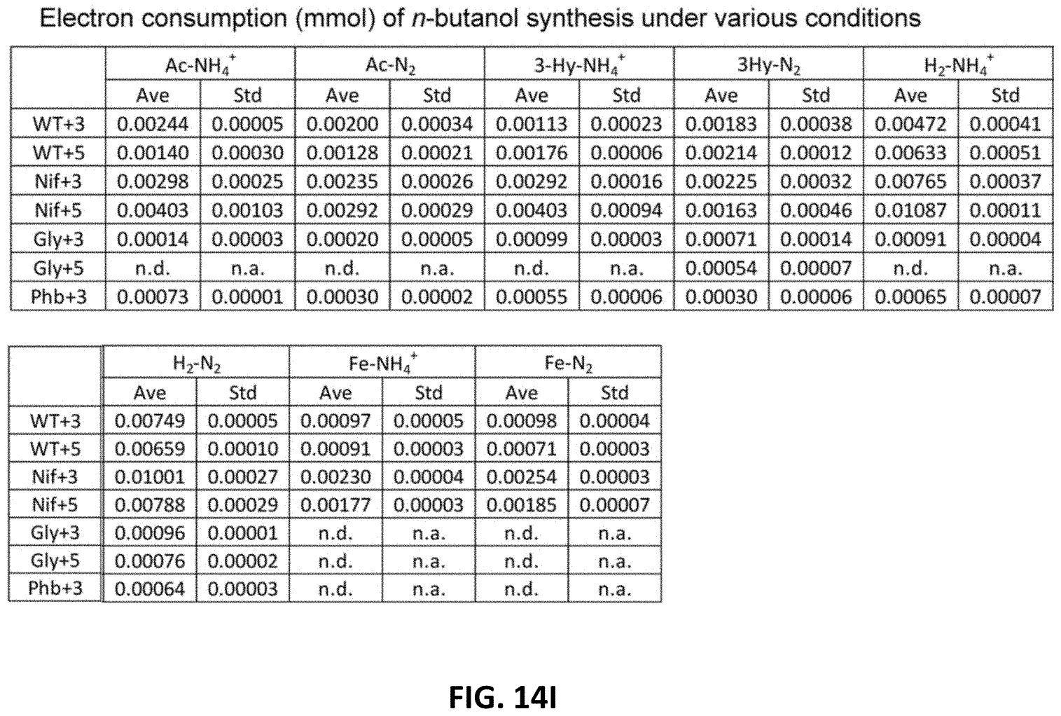

To better understand the distributions of electrons in acetone/CO₂/H₂/biomass synthesis, the electrons consumed by n-butanol biosynthesis, acetone biosynthesis, biomass, CO₂ generation, and H₂ generation were also calculated. For all incubation conditions, biomass is the most significant electron sink (see e.g., FIG. 14A-FIG. 14I). However, under heterotrophic conditions (see e.g., FIG. 14A-FIG. 14D), CO₂ and H₂ generation also act as major electron sinks for most of the strains. The electron consumed by acetone/n-butanol biosynthesis is relatively insignificant compared to biomass, CO₂, and H₂ generation. Under autotrophic conditions (see e.g., FIG. 14E-FIG. 14H), CO₂ was not generated but consumed, hence the electron consumption in generating CO₂ was not considered. Similarly, when H₂ was used as an electron donor, H₂ was consumed, consequently, the electron consumption in generating H₂ was not considered either. Furthermore, no H₂ generation was observed when Fe(II) was used as the electron donor.

Using the same carbon and electron source, the highest ECE was achieved by Nif-3/Nif-5 (0.04±0.01 to 12.47±1.37%), followed by WT-3/WT-5 (0.00 to 6.45±1.73%), Gly-3/Gly-5 (0.00 to 0.05±0.00%), and Phb +3 (0.00 to 0.03±0.01%, see e.g., FIG. 13A-FIG. 13D). In summary, the availability of reducing equivalents due to deletion of an electron-consuming pathway and low cell growth (Nif-3/Nif-5) leads to higher ECE for n-butanol biosynthesis in TIE-1.

n-Butanol Bioproduction can be Achieved with Light, Electricity, and CO₂.

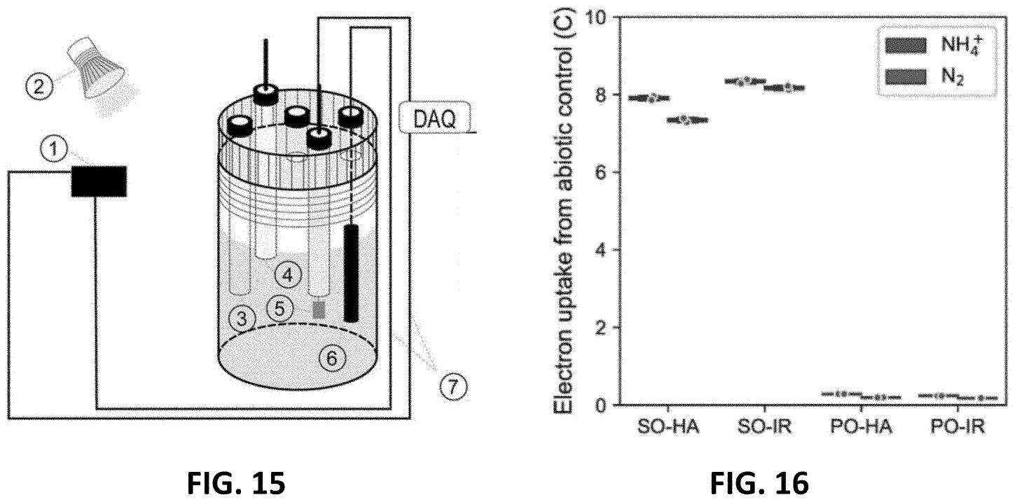

It was recently demonstrated that the photoelectroautotrophic growth of TIE-1 leads to a highly reduced intracellular environment compared to other growth conditions. Under photoelectroautotrophy, TIE-1 can attach to the poised electrode to form a biofilm, and can gain electrons via direct extracellular electron uptake. This process is performed by a complex formed by a single periplasmic decaheme cytochrome c, PioA, an outer membrane porin, PioB, that allows electron transfer across the outer membrane, and PioC a periplasmic electron shuttle. N-butanol production by TIE-1 was investigated under photoelectroautotrophy using a three-electrode sealed BEC (see e.g., FIG. 15). For this experiment, Nif-5 was used as it was the most efficient n-butanol producer under most of the tested conditions (see e.g., FIG. 7C, FIG. 11C, and FIG. 13D).

Four distinct biofuel production BEC platforms were created by combining two different electricity sources (grid-powered potentiostat or a solar panel) with two light sources (infrared or halogen light). In the case of BECs powered by potentiostat, the bioreactors use a three-electrodes system, wherein a reference electrode was used to control the poised potential towards the working electrode steadily at Eₐₚₚₗ 0.5 V. In contrast, for the BECs powered by solar panel, the bioreactors use a two-electrode system, wherein the poised potential was controlled by the existing voltage of the solar panel (Eₐₚₚₗ 0.5 V) from its positive and negative terminals (like voltage obtained in a battery). The different electrode configuration and control modes could lead to significant discrepancies in the reactor performance. For example, using the abiotic control, the BECs powered by solar panels resulted in nearly 25-fold higher electron uptake than the BEC powered by the potentiostat (see e.g., FIG. 16). Further, the potentiostat approach represents conventional electrical sources, while the solar panel approach allows us to leverage renewably generated electricity. Infrared light is only a small portion of the solar spectrum that specifically excites the photosystem of TIE-1. Halogen light mimics natural sunlight that represents the solar spectrum. So, it can excite the photosystem of TIE-1 and support electricity generation by a solar panel simultaneously. BEC platform 1 used solar panel generated electrons and halogen light; BEC platform 2 used solar panel generated electrons and infrared light; BEC platform 3 used potentiostat and halogen light; BEC platform 4 used potentiostat and infrared light. Either N2 or NH₄⁺ was supplied as the nitrogen source. TABLE 7 lists detailed platform setups.

TABLE 7
Incubation combinations used under photoelectroautotrophy.

| | | | Light | Electricity |
|---|---|---|---|---|
| Platforms | | Nitrogen Sources | Sources | Sources |
| 1 | SO—HA—NH₄⁺ | Ammonium | Halogen | Solar panel |
| | | (NH₄⁺) | light | (SO) |
| | SO—HA—N₂ | Dinitrogen gas | (HA) | |
| | | (N₂) | | |
| 2 | SO—IR—NH₄⁺ | Ammonium | Infrared light | |
| | SO—IR—N₂ | Dinitrogen gas | (IR) | |
| 3 | PO—HA—NH₄⁺ | Ammonium | Halogen light | Potentiostat |
| | PO—HA—N₂ | Dinitrogen gas | | (PO) |
| 4 | PO—IR—NH₄⁺ | Ammonium | Infrared light | |
| | PO—IR—N₂ | Dinitrogen gas | | |

Acetone and n-butanol production were measured, and CCE and ECE (measured as coulombic efficiency) were calculated towards n-butanol for each platform. The EECE towards n-butanol was also calculated by dividing the combustion heat of the produced n-butanol by the electrical energy input.

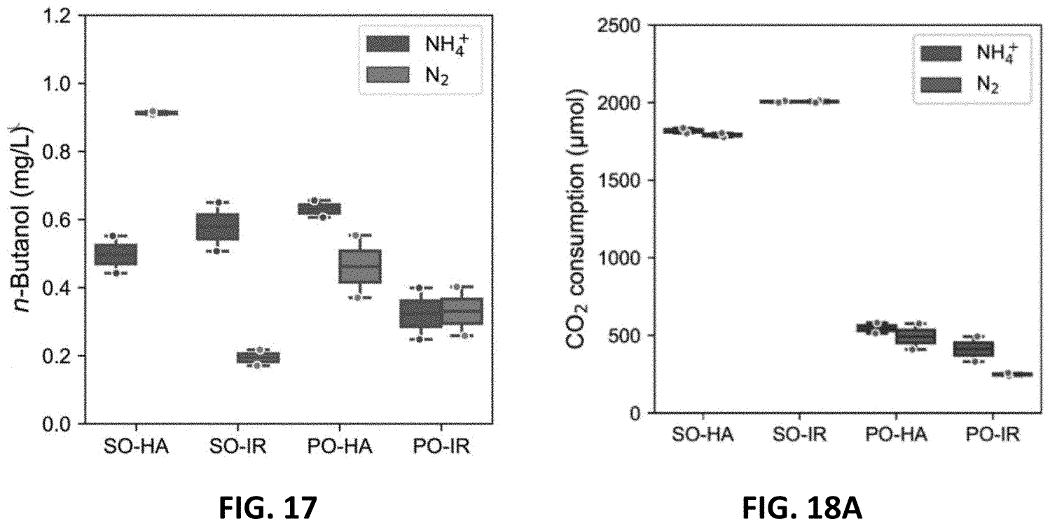

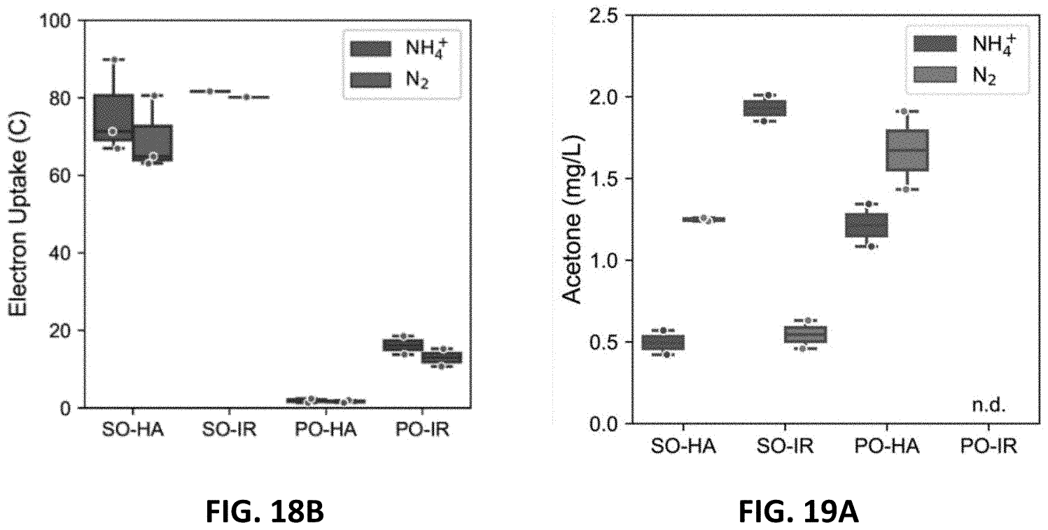

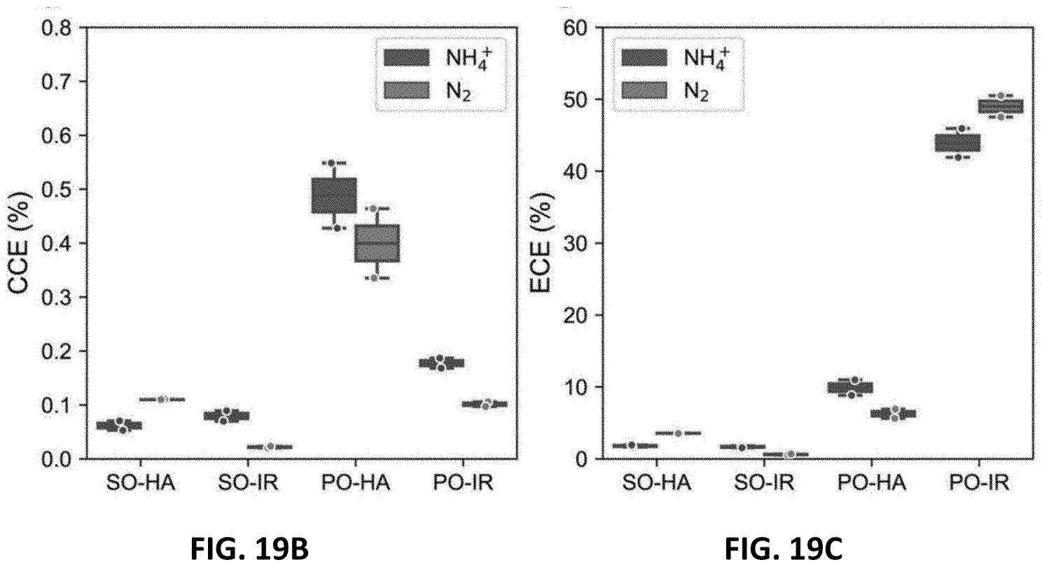

The highest (0.91±0.07 mg/L) and the lowest (0.19±0.02 mg/L) n-butanol production was achieved when N₂ was supplied as a nitrogen source in BEC platform 1 and BEC platform 2, respectively (see e.g., FIG. 17). The BEC platforms powered by solar panels showed 3-8-fold higher CO₂ consumption (see e.g., FIG. 18A) and 5 to 40-fold higher electron uptake (see e.g., FIG. 18B) compared to the BEC platforms powered by the grid-powered potentiostat. Similar to the other autotrophic conditions (see e.g., FIG. 9C-FIG. 9D), little or no acetone was produced (see e.g., FIG. 19A) from the BEC platforms. BEC platform 4 achieved the highest CCE towards n-butanol (0.49±0.06%, see e.g., FIG. 19B) compared to the other three BEC platforms. Although BEC platforms powered by grid-powered potentiostat achieved much lower electron uptake (see e.g., FIG. 18B), they reached a much higher ECE (6-25-folds) than the platforms powered by a solar panel (see e.g., FIG. 19C).

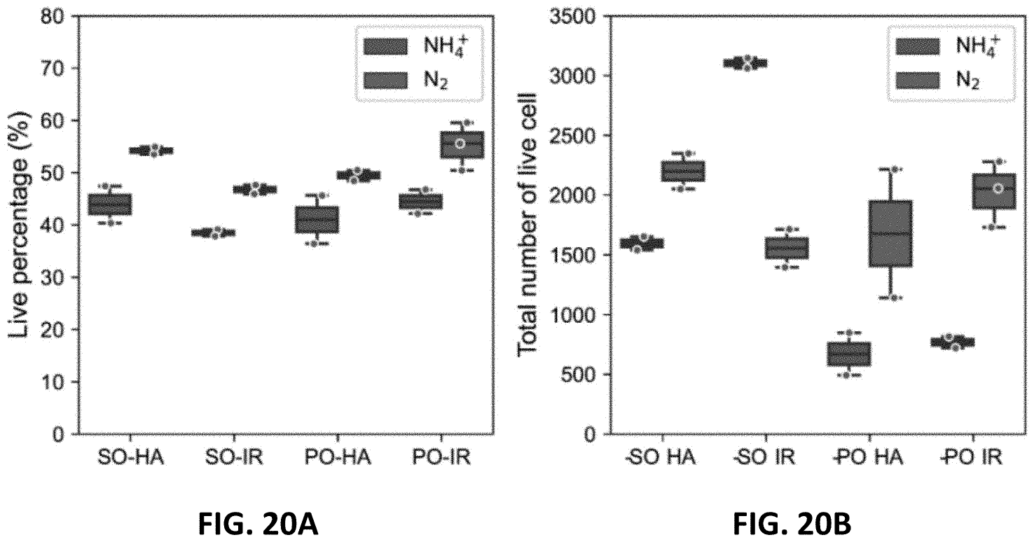

BEC platforms under halogen light achieved higher ECE (4 to 8-fold, except using solar panel incubated with NH₄⁺, see e.g., FIG. 19C) compared to the BEC platforms using infrared light. However, the BEC platforms illuminated by halogen light (platforms 1 and 3) had much lower (20-90%) electron uptake, particularly when using solar panels as an electricity source (see e.g., FIG. 18B). To ensure that a lower number of attached cells did not reduce electron uptake from the platforms using halogen light, a live-dead viability assay was performed. The percentage of live cells attached to the electrodes was similar in all the BEC platforms (40-50%) (see e.g., FIG. 20A-FIG. 20B). This indicates that halogen light is not the ideal light source for TIE-1 with respect to electron uptake.

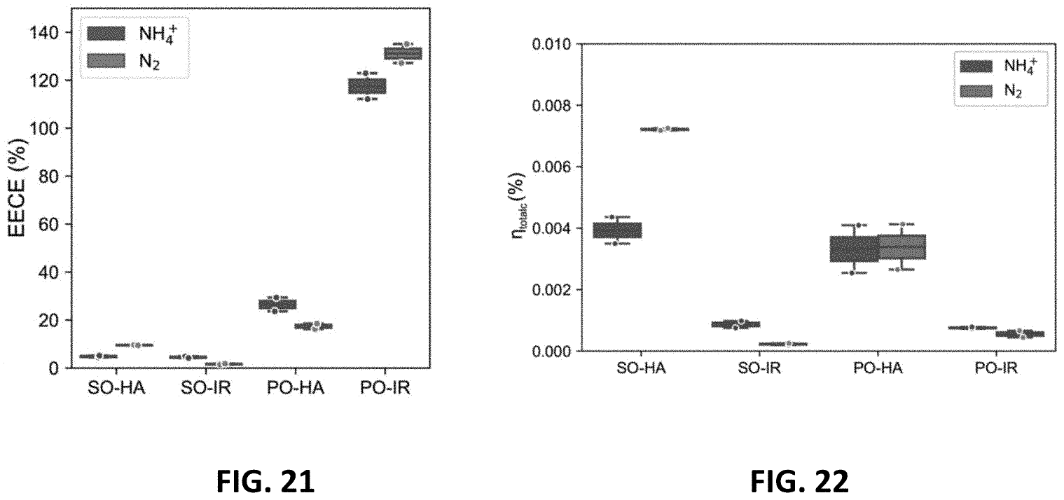

The EECE towards n-butanol was further compared between the two electricity sources. The BEC platforms powered by solar panel showed lower EECE (1.62±0.20 to 9.55±0.34%) than the BEC platforms using a potentiostat (16.62±1.01 to 131.13±3.97%) when the same nitrogen source (either N₂ or NH₄) was supplied (see e.g., FIG. 21). With respect to the light source, platforms using halogen light resulted in higher EECE (4.80±0.38 to 131.14±3.97%) than platforms using infrared light (1.62±0.20 to 26.52±2.87%) when the same nitrogen source was supplied (see e.g., FIG. 21). Halogen light represents the solar spectrum, and several wavelengths from this light source can be absorbed by TIE-1 via the light-harvesting complexes and, eventually, the photosystem. This would lead to higher ATP synthesis via cyclic photosynthesis by TIE-1, perhaps explaining the >100% EECE.

The energy conversion efficiency (ηtotal_{c}) towards n-butanol from light was also calculated for all four systems (see e.g., FIG. 22). Comparing ηtotal_{c} between the two electricity sources (i.e., platforms 1 and 2 vs. 3 and 4), when supplied with the same nitrogen source (either N₂ or NH₄⁺), platforms 2 reached lower ηtotal_{c} than platforms (see e.g., FIG. 22). No significant difference was noticed for ηtotal_{c} between platforms 1 and 3 when incubated with NH₄⁺. However, when incubated with N₂, platform 1 had higher ηtotal_{c} compared to platform 3. As for the light sources, in general, when the same nitrogen source was used, halogen light resulted in lower ηtotal_{c} than infrared light [except using solar panel as an electricity source and incubated with N₂ (see e.g., FIG. 22). These results suggest that TIE-1 prefers infrared light over halogen light.

In summary, BEC platform 1 showed higher n-butanol production, BEC platform 4 showed the highest CCE towards n-butanol, and BEC platform 3 showed the ECE and EECE towards n-butanol. Although BEC platform 1 resulted in moderate conversion efficiencies, the highest n-butanol production (up to five-fold) with the use of sustainable resources (electricity from solar panels and energy from halogen light) make this platform particularly promising for further development as a sustainable and carbon-neutral process for n-butanol production.

### DISCUSSION

In recent years, n-butanol has been proposed as a superior biofuel due to its higher energy content, lower volatility, and reduced hydrophilicity. Herein is described the production of n-butanol by introducing an artificial n-butanol biosynthesis pathway into an anoxygenic photoautotroph Rhodopseudomonas palustris TIE-1. Using metabolic engineering and novel hybrid bioelectrochemical platforms, demonstrated herein is that TIE-1 can produce n-butanol using different carbon sources (organic acids, CO₂), electron sources [H₂, Fe(II), a poised electrode], and nitrogen sources (NH₄+, N2). Interestingly, TIE-1's ability to produce n-butanol under photoelectroautotrophy using light, electricity, and CO₂ may be a stepping-stone for future sustainable solar fuel production.

After introducing a codon-optimized n-butanol biosynthesis pathway in TIE-1 and its mutants (Nif, Gly, and Phb), n-butanol production, acetone production, CCE, and electron conversion efficiency (ECE) towards n-butanol of these constructs was determined under both photoheterotrophic and photoautotrophic conditions. Mutants lacking the nitrogen-fixing pathway (Nif-3/Nif-5) (known to affect redox balance in the cell by consuming reducing equivalents) exhibited a more reduced intracellular environment (indicated by higher CO₂ fixation) and produced more n-butanol compared to WT-3/WT-5. In contrast, deleting acetyl-CoA-consuming pathways (Gly-3/Gly-5 and Phb-3) led to lower n-butanol production. These results show that higher reducing equivalent rather than increased acetyl-CoA availability enhances n-butanol production by TIE-1. These results also agree with previous works where redox balance or reducing equivalent availability plays a vital role in n-butanol production. A closely related strain R. palustris CGA009 has been shown to produce n-butanol when its biosynthesis was the obligate route for maintaining redox balance during photoheterotrophic growth on n-butyrate. Similarly, in E. coli, n-butanol production increased when its biosynthesis acted as an electron-sink to rescue cells from redox imbalance. In addition to the presence of higher reducing equivalents, low cell growth was observed as a factor that may have led to higher titer, efficiency, and productivity. These results agree with a previous study where biomass competes for carbon and electrons with the biosynthesis of n-butanol.

A consistent trend between the 3-gene cassette and 5-gene cassette for n-butanol production was not observed. In a previous study on PHB production by TIE-1, although the expression of phaA and phaB from the TIE-1 genome did not show significant differences, PHB production was different under various growth conditions. This may be due to post-transcriptional differences that likely result in different levels of 3-hydroxybutyryl CoA in the cell. Because 3-hydroxybutyryl-CoA is an intermediate of the n-butanol synthesis, the amount of 3-hydroxybutyryl-CoA may affect n-butanol production. This would likely make it hard to observe any consistent difference between the −3 and −5 strains.

The presence of NH₄⁺ was expected to inhibit the expression of nitrogenase, so nitrogen fixation would not occur, and H₂ would not be produced. However, WT-3/WT-5 and Gly-3/Gly-5 produced H₂ (likely via nitrogenase) despite the presence of NH₄⁺ (see e.g., FIG. 12C). This was in contrast to the Nif-3/Nif-5, which did not produce H₂ under any condition, confirming that the observed H₂ production in the WT and Gly strains is due to nitrogenase activity. The production of H₂ by nitrogenase is well known in CGA00938. This unexpected nitrogenase activity may have been initiated by the lower NH₄⁺ concentrations toward the end of the experiment, which may lead to the induction of nitrogenase gene expression. The Nif mutant does not fix N₂ or produce H₂ via the nitrogenase under both non-nitrogen (i.e., with NH₄⁺) and nitrogen-fixing conditions. This potentially relieves reducing equivalents (NADH) for n-butanol production when compared to the WT and the Gly mutants. In addition, the Nif mutant, when incubated under nitrogen-fixing conditions, is a non-growing strain, and this may also account for the higher n-butanol production in this mutant under these conditions.

By feeding intermediates of n-butanol biosynthesis pathway such as 3Hy as a carbon source, TIE-1 produces more n-butanol (see e.g., FIG. 7A-FIG. 7D). However, despite high n-butanol production, there was a low CCE and low ECE towards n-butanol (see e.g., FIG. 11A-FIG. 11H and FIG. 13A-FIG. 13D), possibly due to higher acetone production (see e.g., FIG. 9A-FIG. 9D). This high acetone production is likely due to the accumulation of acetoacetyl-CoA, converted from 3Hy through 3-hydroxybutyryl-CoA (see e.g., FIG. 6). This acetone production, along with 3Hy being an expensive feedstock compared to CO₂ for bioproduction may be undesirable for economic n-butanol production.

In general, higher n-butanol production, CCE, and ECE towards n-butanol was achieved when acetone production was lower. This is in line with the previous studies where an increase in n-butanol production accompanies a decrease in acetone production. Although using highly reduced substrates, such as glycerol, can increase the ratio of n-butanol to acetone, a significant amount of acetone is always detected while using the n-butanol biosynthesis pathway from C. acetobutylicum. This study addressed this issue by using slow or non-growing cells that produced n-butanol without the production of acetone.

BEC platforms powered by the potentiostat resulted in higher EECE and ECE towards n-butanol. This difference is likely due to the different electron uptake control processes of potentiostat vs. solar panel system. When potentiostat was used, the potential between the cathode and anode was steadily controlled throughout the experiment with respect to the reference electrode. In a solar panel system, the voltage may not be steady, and the current uptake varies with various factors (light intensity, nature of component in the solar system by the manufacture) in addition to the microbial environment. As shown in FIG. 16 and FIG. 18B, the two electricity sources resulted in huge differences in the total posed current on each system. In addition, the electrical or optical losses associated with the solar panel during photoelectron generation could also affect the efficiency. The electrical loss could be due to the limited energy efficiency of the solar panel, which is determined by the diode characteristics and series resistances in the solar panel. And optical loss can be in the form of poor light absorbance or light reflection from the solar cell surfaces or material defects. The platforms with halogen as the light source were found to have higher EECE towards n-butanol (˜8-fold) regardless of the electricity source.

To contextualize the results described herein, CCE, EECE, Eₐₚₚₗ, and n-butanol production were compared with the previous related studies.

EECE. Using solar panel-generated electricity, TIE-1 achieved an EECE towards n-butanol up to 9.54%, which increased by over 13-fold (up to 131.13%) when grid-based electricity was used (see e.g., FIG. 21). In a previous study using a hybrid water-splitting system, R. eutropha achieved an EECE of 16% towards C₄+C₅ alcohol using grid-based electricity. These data suggest that TIE-1 can also achieve higher EECE using grid-based electricity.

Eappl and power requirement. TIE-1 can gain electrons directly from an electrode, which requires lower Eₐₚₚₗ for photoautotrophic growth and n-butanol biosynthesis (Eₐₚₚₗ=0.1-0.5 V). In contrast, the hybrid water-splitting system used to synthesize C3-C5 alcohol or PHB by R. eutropha used an Eₐₚₚₗ of 2.0 V. Similarly, n-butanol synthesis by Clostridium sp. using MES used an Eₐₚₚₗ of 0.8 V. Assuming that all the reactors use 1 mA of current, the power would be 5×10⁻⁴ W for n-butanol bioproduction by TIE-1. In contrast, R. eutropha would require 2×10⁻³ W for water-splitting, and Clostridium sp. would require 8×10⁻⁴ W. Therefore, TIE-1 uses four times less power than R. eutropha and 1.6 times less power than Clostridium sp. This implies that even low-efficiency solar panel-based platforms and low sunlight conditions can be easily used for bioproduction using TIE-1.

n-Butanol production. Under photoelectroautotrophy, TIE-1 produced 0.91±0.07 mg/L of n-butanol in 10 days (see e.g., FIG. 17). Clostridium sp. produced 135 mg/L n-butanol in 35 days. Compared to R. eutropha and Clostridium sp., the platform described herein produced lower n-butanol. Under photoautotrophic conditions, TIE-1 produced a maximum of 3.09±0.25 mg/L of n-butanol in batch culture (see e.g., FIG. 7C). Initial studies in cyanobacteria resulted in 2.2 mg/L. Recently, using a modular engineering method, cyanobacteria produced 4.8 g/L of n-butanol, which is 2000-fold higher than the initial n-butanol production described herein. With future engineering efforts, the n-butanol production efficiency of TIE-1 may be increased.

CCE. Thus far, no autotrophic n-butanol production study has reported CO₂ consumption. Herein TIE-1's CCE towards n-butanol was compared with that reported for heterotrophic production. Although most heterotrophic growth media use yeast extract (an undefined carbon source), for simplicity, the CCE calculations considered the total amount of sugar as the only carbon source for E. coli and yeast from the previous studies. The early trials in E. coli and S. cerevisiae reached CCEs of 0.11 and 0.02%. As yeast extract also provides carbon, the real CCEs from these studies should be even lower. With intensive metabolic engineering, the CCE towards n-butanol reached up to 45.92% in E. coli and 11.52% in S. cerevisiae (calculated from the reported g/g yield). The results herein show that TIE-1 may have the CCE (mol/mol) of up to 4.58±0.21% and 1.95±0.26% under photoautotrophic and photoheterotrophic conditions, respectively (see e.g., FIG. 11A-FIG. 11H). This is 20 and 200 times higher than that of initial studies in E. coli and S. cerevisiae. Photoautotrophic bioproduction is superior due to the low cost of CO₂ compared to heterotrophic substrates. Thus, developing TIE-1 further via metabolic engineering, synthetic biology, and bioprocess engineering may make it an economically viable bioproduction platform.

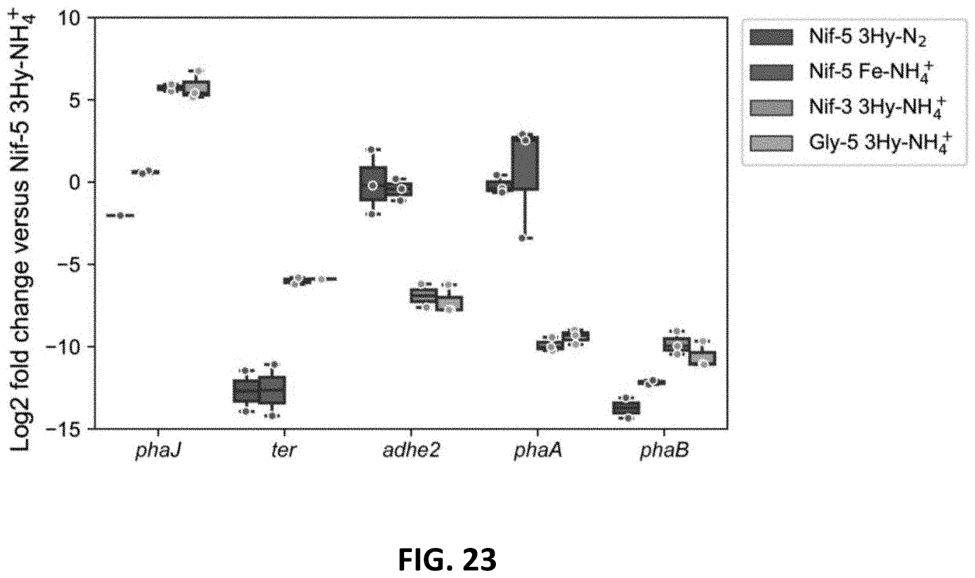

In summary, TIE-1 can achieve high EECE and CCE towards n-butanol with lower power input while producing an amount of n-butanol comparable to the initial studies in established bioproduction chassis organisms like E. coli and S. cerevisiae. This study represents the initial effort of producing carbon-neutral fuels using TIE-1. Although the production is relatively low compared to other model organisms, a number of modifications may be made to improve the n-butanol titer. For example, an increased expression of genes in the n-butanol biosynthesis pathway from Nif-5 incubated with 3Hy (the strain and condition that resulted in the highest n-butanol production) was observed (see e.g., FIG. 23). Therefore, increasing gene expression by driving each gene in the n-butanol biosynthesis pathway with its own promoter may increase n-butanol production. Because only a small portion of electrons go toward n-butanol synthesis, deleting more pathways that consume electrons may make n-butanol a more significant electron sink. Also, increasing intracellular iron may lead to higher cytochrome production, which would increase electron uptake. Furthermore, creating a BEC platform with built-in solar conversion to electricity capability may reduce electrical energy loss. Finally, higher electron uptake, which should be beneficial for n-butanol synthesis, may be achieved by using nanoparticle-modified electrodes. Taken together, TIE-1 offers a sustainable route for carbon-neutral n-butanol biosynthesis and other value-added products. As CO₂ concentrations are rising in the atmosphere, such bioproduction strategies need immediate attention and support.

Methods

Bacterial Strains, Media, and Growth Conditions.

All strains used in this study are listed in TABLE 8.

TABLE 8
Strains used in this study.

| | Relevant genotypes of | |
|---|---|---|
| Strains | R. palustris TIE-1 | Plasmid |
| AB437 | Wild type (WT) | None |
| AB647 | ΔphaC1ΔphaC2 (Phb) | None |
| AB133 | ΔnifA1ΔnifA2 (Nif) | None |
| AB145 | ΔglgA (Gly) | None |
| AB147 | Wild type (WT) | pAB675 |
| AB153 | ΔphaC1ΔphaC2 (Phb) | pAB675 |
| AB149 | ΔnifA1ΔnifA2 (Nif) | pAB675 |
| AB151 | ΔglgA (Gly) | pAB675 |
| AB148 | Wild type (WT) | pAB744 |
| AB150 | ΔnifA1ΔnifA2 (Nif) | pAB744 |
| AB152 | ΔglgA (Gly) | pAB744 |

E. coli strains were grown in lysogeny broth (LB; pH 7.0) at 37° C. For aerobic growth, Rhodopseudomonas palustris TIE-1 was grown at 30° C. in YP medium (3 g/L yeast extract, 3 g/L peptone) supplemented with 10 mM MOPS [3-N (morpholino) propanesulphonic acid] (pH 7.0) and 10 mM succinate (YPSMOPS) under the illumination of an infrared LED (880 nm). For growth on a solid medium, YPSMOPS or LB was supplemented with 15 g/L agar. For anaerobic phototrophic growth, TIE-1 was grown in anoxic bicarbonate buffered freshwater (FW) medium. All FW media was prepared under a flow of 34.5 kPa N₂+CO₂ (80%, 20%) and dispensed into sterile anaerobic Balch tubes. The cultures were incubated at 30° C. in an environmental chamber fitted with an infrared LED (880 nm). For photoheterotrophic growth, the FW medium was supplemented with 50 mM MOPS at pH 7.0 and sodium 3-hydroxybutyrate or sodium acetate at pH 7.0, to a final concentration of 50 mM. For photoautotrophic growth on iron, anoxic sterile stocks of FeCl₂ and nitrilotriacetic acid (NTA) were added to reach final concentrations of 5 mM and 10 mM, respectively. For photoautotrophic growth on H₂, TIE-1 was grown in FW medium at pH 7.0 and 12 psi of 80% H₂/20% CO₂. For all carbon and electron sources, either ammonium chloride (5.61 mM) or dinitrogen gas (8 psi) was supplied as nitrogen source. All sample manipulations were performed inside an anaerobic chamber with a mixed gas environment of 5% H₂/75% N₂/20% CO₂ (Coy Laboratory, Grass Lake, Mich.). When needed, 400 μg/mL kanamycin was added for TIE-1, and 50 μg/mL kanamycin was added for E. coli.

R. palustris TIE-1 Deletion Mutant Construction.

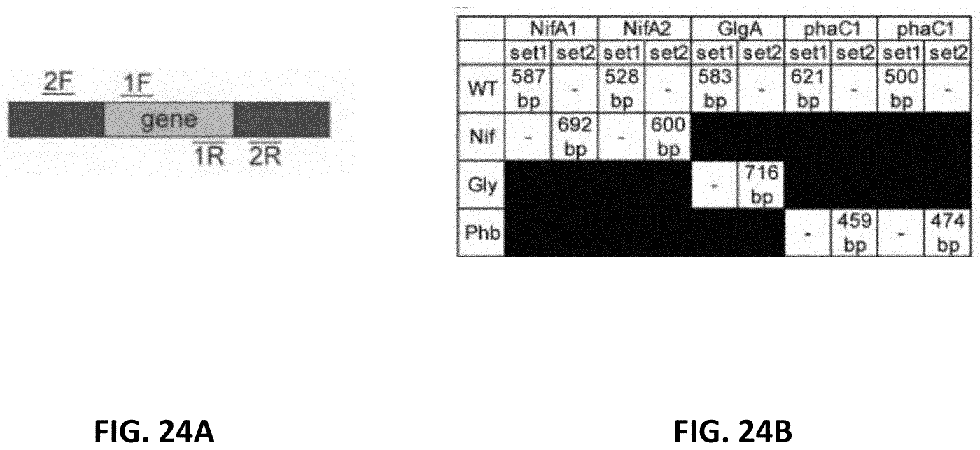

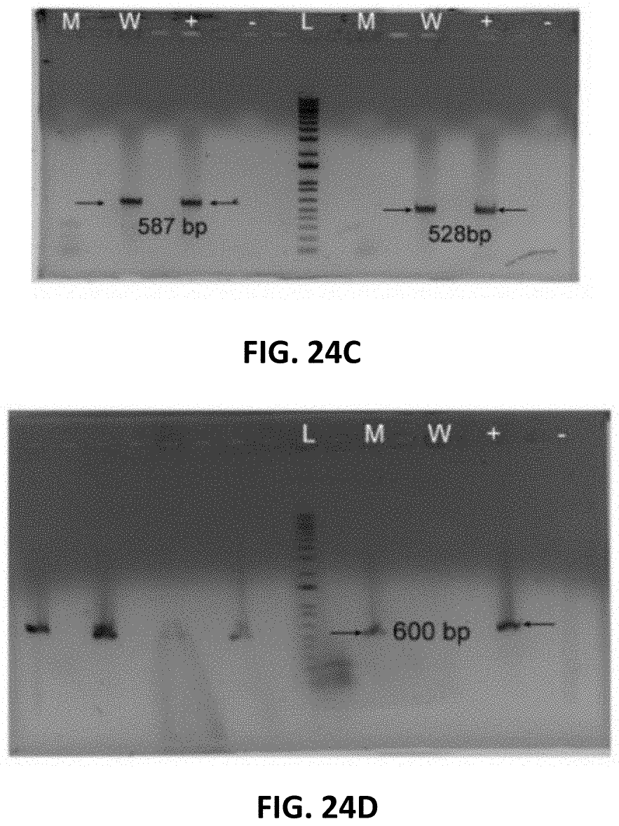

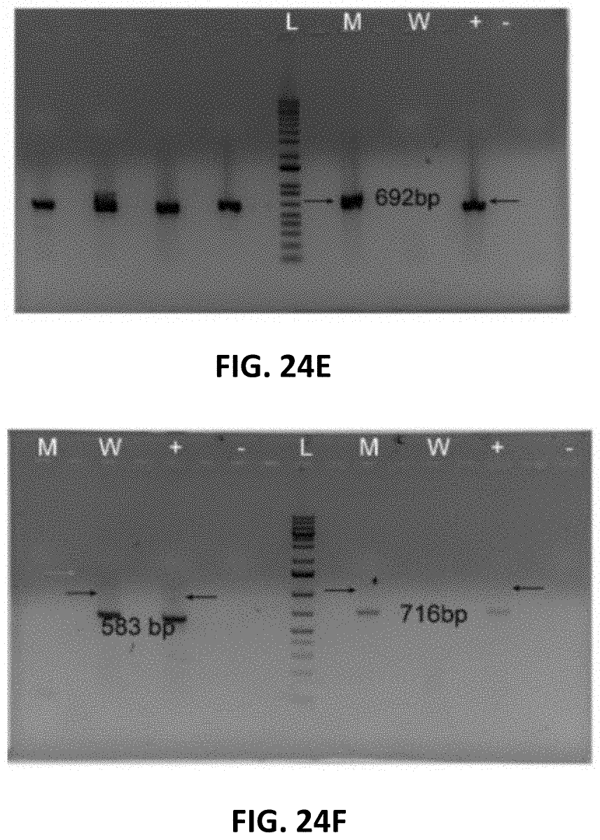

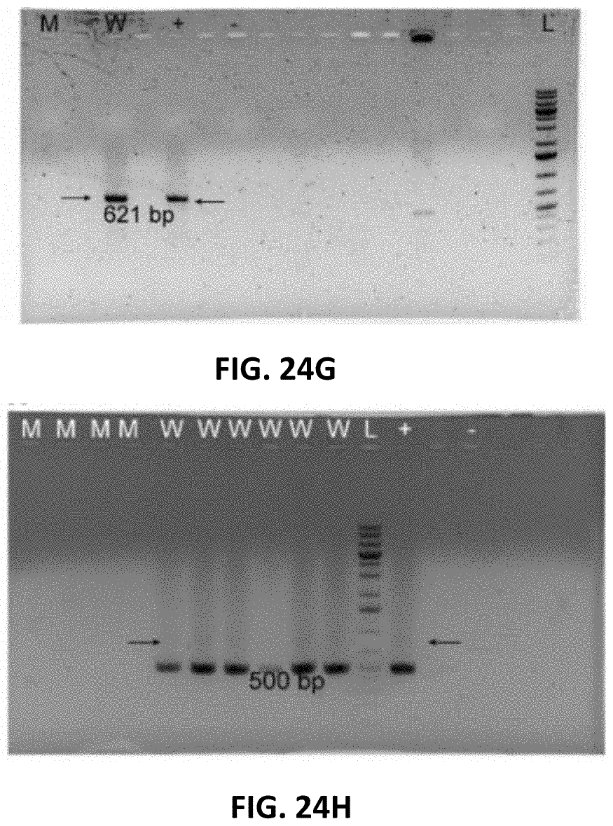

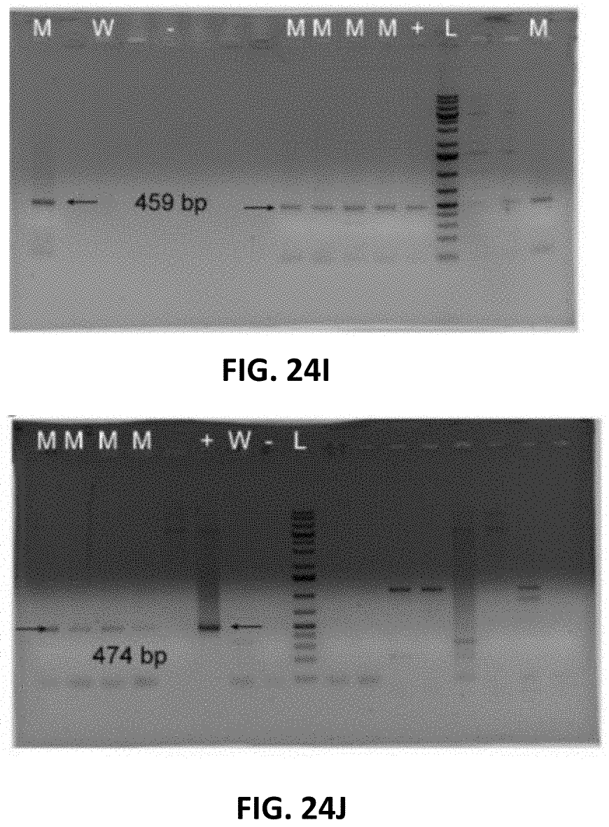

Three mutants were constructed, two of which were double mutants using the method described in a previous study. Respectively, Glycogen synthase knockout was created by deleting Rpal_0386 (glgA), nitrogenase knockout was created by deleting Rpal_5113 (nifA1), and Rpal_1624 (nifA2), and hydroxybutyrate polymerase knockout was created by deleting Rpal_2780 (phaC1) and Rpal_4722 (phaC2). Briefly, the 1 kb upstream and 1 kb downstream regions of the gene were PR amplified from the R. palustris TIE-1 genome, then the two homology arms of the same gene were cloned into pJQ200KS plasmid. The resulting vector was then electroporated into E. coli and then conjugated to R. palustris TIE-1, using the mating strain E. coli S17-1/λ. After two sequential homologous recombination events, mutants were screened by PCR, as shown in FIG. 24A-FIG. 24J. The primers used for mutant construction and verification are listed in TABLE 9 and TABLE 10.

TABLE 9
Primers for constructing and sequencing the
plasmids used for generating mutants.

| Primer Name | Primer Sequence |
|---|---|
| RpaI_51131 kbDnFwXbaI | CATACTCTAGAAGCAGATCATCGTGGTGTCTTG |
| | (SEQ ID NO: 1) |
| RpaI_51131 kbDnBamHIRev | ATCAGGATCCCGCGGTCTCGGTGACCAGCTC |
| | (SEQ ID NO: 2) |
| RpaI_51131 kbUpFwSacI | TCATGAGCTCCAGAAGACGCTGGTGCTGAC |
| | (SEQ ID NO: 3) |
| RpaI_51131 kbupRevXbaI | GACTCTAGACATAGCTGGTCTCCATCGCTC |
| | (SEQ ID NO: 4) |
| RpaI_1624 1 Kb UpSpeI | TAGACTACTAGTCGTTTACAGCTCCGATCCGAATG |
| Fw NifA | (SEQ ID NO: 5) |
| RpaI_1624 1 Kb Up NifA | ATACTAGGATCCGTCGGCTTCAGGACATGGTCG |
| BamHI Rev | (SEQ ID NO: 6) |
| RpaI_16241 kbDnXbaI Fw | CAGCTCTAGAATCATCAGACAAGGCGCGAC |
| | (SEQ ID NO: 7) |
| RpaI_16241 kbDnBamHIIRev | TCATGGATCCATTGGCAAGCGCATCACCCGGACC |
| | (SEQ ID NO: 8) |
| RpaI_2780 1 Kb Up Fw | CATATGACTAGTGAGTGTCCTTCAGCTTCTCCAGGA |
| | (SEQ ID NO: 9) |
| RpaI_2780 1 Kb Up Rev | CATATGGGATCCGAATCAACACTACAGTCCGGT |
| | (SEQ ID NO: 10) |
| RpaI_2780 1 Kb Dn Fw | CATATGGGATCCTGAACGACGCGCGGCGGCGAAGC |
| | (SEQ ID NO: 11) |
| RpaI_2780 1 Kb Dn Rev | CATATGCCCGGGACGGTGAGCACCGAATTCGCCTG |
| | (SEQ ID NO: 12) |
| RpaI_4722 1 Kb Up Fw | CATATGGCGGCCGCCGCGTGTCGTCTCAGCATTGCG |
| | (SEQ ID NO: 13) |
| RpaI_4722 1 Kb Up Rev | CATATGGGATCCCATCACCTCGTCGCGGCCGTC |
| | (SEQ ID NO: 14) |
| RpaI_4722 1 Kb Dn Fw | CATATGGGATCCTGAGCGGTCTGCGGCAACGCCGC |
| | (SEQ ID NO: 15) |
| RpaI_4722 1 Kb Dn Rev | CATATGCTGCAGTGGCCGACGACACCAACGAGCT |
| | (SEQ ID NO: 16) |
| Gly (RpaI_0386) UP XbaI F | GCTATATCTAGAAGCGCAACGAGAGCTTCGACATTCTGCC |
| | (SEQ ID NO: 17) |
| Gly (RpaI0386) UP BamHI R | ATATATGGATCCGCGTCGAGCTTCTCGATCATCGC |
| | (SEQ ID NO: 18) |
| Gly (RpaI_0386) DN BamHI F | ATATATGGATCCCAAGCTGCGCACATCATCCCA |
| | (SEQ ID NO: 19) |
| Gly (RpaI_0386) DN XhoI R | GCATATCTCGAGTCTGATCATGGAGCCGGCTTG |
| | (SEQ ID NO: 20) |
| Seq gly (RpaI_0386) F | GAGAACACTGTGGTGCT |
| | (SEQ ID NO: 21) |

TABLE 10
Primers used for checking the mutants.

| Primer Name | Primer Sequence |
|---|---|
| PCR check RpaI_5113 | GAGTGCTGACCTGAGCGAATAG |
| set 1 F | (SEQ ID NO: 22) |
| PCR check RpaI_5113 | CACTTGGTCGCGTCGATCACATAG |
| set 1R | (SEQ ID NO: 23) |
| PCR check RpaI_5113 | CGTTCGCACTTCCGGATGGAC |
| set 2 F | (SEQ ID NO: 24) |
| PCR check RpaI_5113 | CTTGATGGTCTGGTTGCCGCCGAC |
| set 2 R | (SEQ ID NO: 25) |
| PCR check RpaI_1624 | GAACCAGCTCGCGATCCATCTCAG |
| set 1 F | (SEQ ID NO: 26) |
| PCR check RpaI_1624 | GCCACGATGTAGACTTCCTGTGCCTTG |
| set 1R | (SEQ ID NO: 27) |
| PCR check RpaI_1624 | GAGCACCTTCTGGGCGAGCACGATC |
| set 2 F | (SEQ ID NO: 28) |
| PCR check RpaI_1624 | ATGCCGATGGCCCAAAATTCCCG |
| set 2 R | (SEQ ID NO: 29) |
| PCR check RpaI_2780 | CTCGCAACAATCGTCGCACTC |
| set 1 F | (SEQ ID NO: 30) |
| PCR check RpaI_2780 | AAGGATTGGCCTATACCG |
| set 1R | (SEQ ID NO: 31) |
| PCR check RpaI_2780 | CTTCCAGAACGAAATCATGCAGCTC |
| set 2 F | (SEQ ID NO: 32) |
| PCR check RpaI_2780 | GGTGGCGTCGGAATTCCAGT |
| set 2 R | (SEQ ID NO: 33) |
| PCR check RpaI_4722 | ATCCGCGGCTTGAGCAAGG |
| set 1 F | (SEQ ID NO: 34) |
| PCR check RpaI_4722 | TTGGCATCCCATTCCACG |
| set 1R | (SEQ ID NO: 35) |
| PCR check RpaI_4722 | GTCAAACTTCGCCCTCACCAATC |
| set 2 F | (SEQ ID NO: 36) |
| PCR check RpaI_4722 | CGAGGCAATAGCCGACCG |
| set 2 R | (SEQ ID NO: 37) |
| PCR check RpaI_0386 | TCACTTCGACAAGTCGTGCC |
| set 1 F | (SEQ ID NO: 38) |
| PCR check RpaI_0386 | TAATCGTGACGATGGTCACC |
| set 1 R | (SEQ ID NO: 39) |
| PCR check RpaI_0386 | AGGTCCACAGCTTCAACGAG |
| set 2 F | (SEQ ID NO: 40) |
| PCR check RpaI_0386 | GTGTCGATGCCGTTGAGGAT |
| set 2 R | (SEQ ID NO: 41) |

Plasmid Construction.

All plasmids used in this study are listed in TABLE 11.

TABLE 11
Plasmids used in this study.

| Plasmid | Construct |
|---|---|
| pAB423 | Empty Vector |
| pAB675 | phaJ, ter, adhE2 in pAB423 |
| pAB744 | phaJ, ter, adhE2, phaA, phaB in |
| | pAB423 |

There are five genes involved in the n-butanol biosynthesis: phaJ, ter, adhE2, phaA, and phaB (see e.g., FIG. 1A). Among these five genes, TIE-1 has homologs of the first two (phaA and phaB). Hence, two different cassettes were designed, namely, a 3-gene cassette (3-gene), which has phaJ, ter, adhE2, and a 5-gene cassette (5-gene), which has the 3-gene plus a copy of the phaA-phaB operon from TIE-1. phaJ, ter, and adhE2 sequences were obtained from published studies. The phaJ gene, isolated from Aeromonas caviae, was chosen because it codes for an enzyme that has a higher specificity for its substrate. The ter gene isolated from Euflena gracilis was selected because it is unable to catalyze the reverse oxidation of butyryl-CoA. The adhE2 gene isolated from C. acetobutylicum is chosen because the enzyme it encodes for specifically catalyzes the reduction of the butyryl-CoA. All three foreign genes (phaJ, ter, and adhE2) were codon-optimized by Integrated DNA Technology (IDT) for TIE-1. The cassette was synthesized as G-blocks by IDT, which were then stitched together by overlap extension and restriction cloning. The phaJ-ter-adhE2 cassette was then inserted into plasmid pRhokS-2, resulting in pAB675. PhaA and phaB were amplified as an operon from the R. palustris TIE-1 genome. The phaA-phaB cassette was then cloned into pAB675 to obtain pAB744. Upon obtaining mutants and plasmids, either the 3-gene or the 5-gene was conjugated into WT TIE-1 or the mutants, using mating the strain E. coli S17-1/λ. The primers used for cassette construction are listed in TABLE 12. The primers used for cassette sequencing are listed in TABLE 13.

TABLE 12
Primers for constructing the 3-gene
and the 5-gene cassette.

| Primer | |
|---|---|
| Name | Primer Sequence |
| 1F | ATCGAATTCCGCTAGCTTCACGCTGCCGCA |
| | (SEQ ID NO: 42) |
| 12F | GACCCGGCGTTTGCGGCGACCACGGCGTTC |
| | (SEQ ID NO: 43) |
| 23F | GTCGCGCATCACCGCCGCCTTCGGCTACGG |
| | (SEQ ID NO: 44) |
| 34F | ACTCGTATATCGGCCCGGAAGCGACCCAGG |
| | (SEQ ID NO: 45) |
| 45F | GACCGCATCTAAATGCATGCAGGATGAGGA |
| | (SEQ ID NO: 46) |
| 56F | GGACGCCGCCGTGAAGGCCGGCGCGCCGAA |
| | (SEQ ID NO: 47) |
| 67F | TGCAGTCCGTCGAGAAGTCGGAGCTGTTCA |
| | (SEQ ID NO: 48) |
| 78F | CTGTTCAAGCTGGGCTACGTCAACAAGATC |
| | (SEQ ID NO: 49) |
| 89F | CCACGCGATCGAGGCGTATGTGTCGGTGAT |
| | (SEQ ID NO: 50) |
| 12R | GAACGCCGTGGTCGCCGCAAACGCCGGGTC |
| | (SEQ ID NO: 51) |
| 23R | CCGTAGCCGAAGGCGGCGGTGATGCGCGAC |
| | (SEQ ID NO: 52) |
| 34R | CCTGGGTCGCTTCCGGGCCGATATACGAGT |
| | (SEQ ID NO: 53) |
| 45R | TCCTCATCCTGCATGCATTTAGATGCGGTC |
| | (SEQ ID NO: 54) |
| 56R | TTCGGCGCGCCGGCCTTCACGGCGGCGTCC |
| | (SEQ ID NO: 55) |
| 67R | TGAACAGCTCCGACTTCTCGACGGACTGCA |
| | (SEQ ID NO: 56) |
| 78R | GATCTTGTTGACGTAGCCCAGCTTGAACAG |
| | (SEQ ID NO: 57) |
| 89R | ATCACCGACACATACGCCTCGATCGCGTGG |
| | (SEQ ID NO: 58) |
| 10R | CGATCGATCGATCGATCTGCAGCTCCAAAA |
| | (SEQ ID NO: 59) |
| 910F | TCTACAACACCCTCGACAAGATGAGCGAGC |
| | (SEQ ID NO: 60) |
| 910R | CGATCGATCGATCGATCTGCAGCTCCAAAA |
| | (SEQ ID NO: 61) |
| But XbaI | CCGAGTCTAGACGTTTCATATGTCCGC |
| NdeI F | (SEQ ID NO: 62) |
| But EcoRI | GCGCGAATTCTTAGATGCGGTCGAAGC |
| R | (SEQ ID NO: 63) |
| But EcoRI | GCATGAATTCAGGATGAGGATCGTTTCGCATGAAGGT |
| F | (SEQ ID NO: 64) |
| But KpnI R | GTTAGGTACCGATCGATCGATCCATCTGCAGCTCC |
| | (SEQ ID NO: 65) |
| phaA | ATCGGCAAGCTTCTAACCAGGAGATGTCCATGTCGGA |
| HindIII F | (SEQ ID NO: 66) |
| phaB PstI | ATATCTGCAGTCAAACCATGTATTGGCCGCCGTTGAT |
| R | GGTGA |
| | (SEQ ID NO: 67) |

TABLE 13
Primers for sequencing the 3-gene
and the 5-gene cassette.

| Primer Name | Primer Sequence |
|---|---|
| But seq 2 | GCGAGGACAAGCCAATCGCCACCCTCACCACCC |
| | GCATC |
| | (SEQ ID NO: 68) |
| But seq 3 | GCCTACTCGTATATCGGCCCGGAAGCGACCCAG |
| | GCCCTC |
| | (SEQ ID NO: 69) |
| But seq new 2 | AGTCACGGCCGAGGTGGAAG |
| | (SEQ ID NO: 70) |
| But Seq new 3 | GCGTCCTGAAGCCGTTCG |
| | (SEQ ID NO: 71) |
| But Seq 4 | CGGCATCATCGACCACGACGACAGCCTCGGCAT |
| | CACCAAG |
| | (SEQ ID NO: 72) |
| But Seq 5 | CGGGCCATACCTCGTCGCTGTATATCGACAGCC |
| | AGAAC |
| | (SEQ ID NO: 73) |
| phaA phaB Seq2 | TGGTGCTGATGACCGCCAA |
| | (SEQ ID NO: 74) |
| phaA phaB seq3 | TGGGACGTGAGTTCGTTCGA |
| | (SEQ ID NO: 75) |

Substrate Measurement.

Substrate concentrations at the beginning (T₀) and the end (T_{f}) were measured to calculate carbon and electron conversion efficiency to n-butanol. The incubation time of each experiment can be found in TABLE 3

CO₂ and H₂ Analysis by Gas Chromatography.

CO₂ and H₂ were analyzed using a method described in a previous study. Gas samples were analyzed using gas chromatography (Shimadzu BID 2010-plus, equipped with Rt®-Silica BOND PLOT Column, 30m×0.32 mm; Restek, USA) with helium as a carrier gas. To measure the CO₂ content of the liquid phase, 1 mL of the cell-free liquid phase was added to 15 mL helium-flushed septum-capped glass vials (Exetainer, Labco, Houston) containing 1 mL 85% phosphoric acid. Then 40 μL of the resulting gas from the Balch tube was injected into the Shimazu GC-BID, using a Hamilton™ gas-tight syringe. To measure the CO₂ and H₂ contents of the gas phase, either 40 μL of the gas phase was directly injected into the Shimadzu GC-BID, or 5 mL of the gas phase was injected into a 15 mL helium-flushed septum-capped glass vial (Exetainer, Labco, Houston), using a Hamilton™ gas-tight syringe. Then 50 μL of the diluted gas sample was injected into the Shimazu GC-BID, using a Hamilton™ gas-tight syringe. A standard curve was generated by the injection of 10 μL, 25 μL, and 50 μL of H₂+CO₂ (80%, 20%). The total moles of CO₂ in the reactors were calculated using the ideal gas law (PV=nRT).

Organic Acid Analysis by Ion Chromatography.

For measuring organic acid concentration, after 1:50 dilution, the acetate and 3-hydroxybutyrate concentrations at the starting and endpoint of culture for each sample were quantified using an Ion Chromatography Metrohm 881 Compact Pro with a Metrosep organic acid column (250 mm length). Eluent (0.5 mM H₂SO₄ with 15% acetone) was used at a flow rate of 0.4 mL min⁻¹ with suppression (10 mM LiCl regenerant).

Ferrous Iron [Fe(II)] Analysis by Ferrozine Assay.

The Fe(II) concentration measurement was done using 10 μL of culture mixed with 90 μL 1 M HCl in a 96-well plate inside the anaerobic chamber (filled with 5% H₂/75% N₂/20% CO₂, Coy Laboratory, Grass Lake, Mich.). After the plate was removed from the anaerobic chamber, 100 μL of ferrozine (0.1% (w/v) ferrozine in 50% ammonium acetate) was added to the sample. Then the 96-well plate was covered with foil and incubated at room temperature for 10 min. before the absorbance was measured at 562 nm. The absorbance was then converted to Fe(II) concentration based on a standard curve generated by measuring the absorbance from 0 mM, 1 mM, 2.5 mM and, 5 mM Fe(II).

In Vivo Production of n-Butanol.

The plasmids with the n-butanol pathway were unstable when adapting the strain to the nitrogen-fixing or photoautotrophic conditions. To avoid this problem, a twice-washed heavy inoculum from YPSMOPS was used under all conditions.

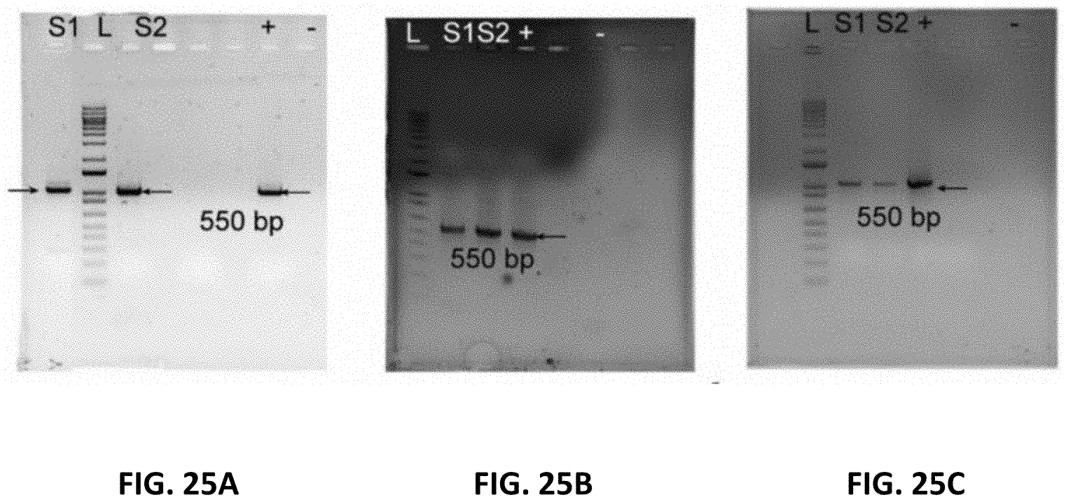

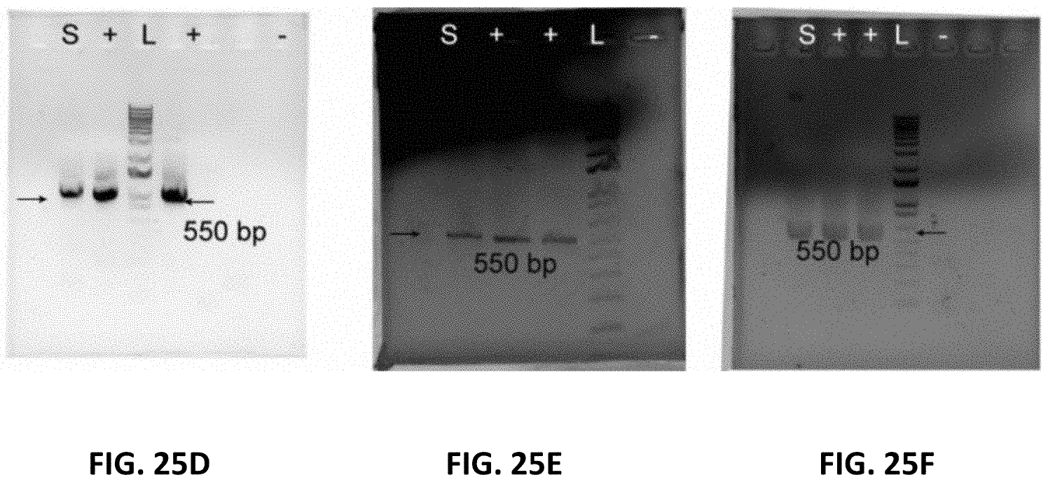

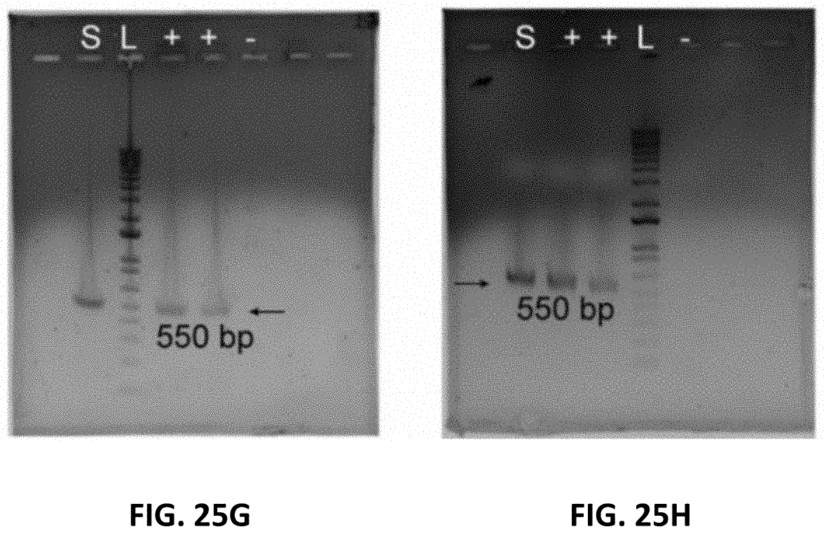

All strains were inoculated in 50 mL of YPSMOPS with kanamycin with a 1:50 dilution from a pre-grown culture. When the OD660 reached 0.6-0.8, the culture was inoculated into 300 mL of YPSMOPS with kanamycin. When the OD660 reached 0.8˜1, 10 mL of culture was saved for a PCR check (see e.g., FIG. 25A-FIG. 25H). The rest of the culture was washed twice with ammonium-free FW medium and resuspended using anoxic ammonium-free FW medium inside the anaerobic chamber. Finally, the culture was inoculated into the medium containing different carbon sources and electron donors (acetate, 3-hydroxybutyrate, H₂, Fe(II), or electrode) in either a sealed Balch tube (initial OD660˜1) or a BEC (initial OD660˜0.7). The tubes and the reactors were sealed throughout the process, and samples were taken after the cultures reached the stationary phase (incubation time listed in TABLE 3), using sterile syringes.

Extraction and Quantification of n-Butanol and Acetone.

After the culture entered the late stationary phase, 1 mL of culture was removed from the culture tube using a syringe and centrifuged at 21,100×g for 3 min. The supernatant was then filtered using a syringe filter, and the filtrate or the standard was extracted with an equal volume of toluene (containing 8.1 mg/L iso-butanol as an internal standard) and mixed using a Digital Vortex Mixer (Fisher) for 5 min. followed by centrifugation at 21,100×g for 5 min. After centrifugation, 250 μL of the organic layer was added to an autosampler vial with an insert. The organic layer was then quantified with GC-MS (Shimazu GCMS-QP2010 Ultra), using the Rxi®−1 ms column. The oven was held at 40° C. for 3 min, ramped to 165° C. at 20° C./min, then held at 165° C. for 1 min. Samples were quantified relative to a standard curve for 0, 0.2025, 0.405, 0.81, 2.025, 4.05, and 8.1 mg/L of n-butanol and 0, 0.784, 3.92, 7.84, 39.2, 78.4, and 392 mg/L of acetone. An autosampler was used to reduce the variance of injection volumes.

Bioelectrochemical Platforms and Growth Conditions.

A three-electrode sealed-type bioelectrochemical cell (BEC, C001 Seal Electrolytic Cell, Xi'an Yima Optoelectrical Technology Com., Ltd, China) containing 80 mL of FW medium was used for testing n-butanol production. The three electrodes were configured as a working electrode (a graphite rod, 3.2 cm²), a reference electrode (Ag/AgCl in 3.5M KCl), and a counter electrode (Pt foil, 5 cm²). FW medium (76 mL) was dispensed into sterile, sealed, three-electrode BECs, which were bubbled for 60 min. with N₂+CO₂ (80%/20%) to remove oxygen and pressurized to ˜7 psi. Four BECs were operated simultaneously (n=3 biological replicates) with one no-cell control. All photoelectroautotrophic experiments were performed at 26° C. under continuous infrared light (880 nm) or halogen light. The electrical potential of 0.5 V (Eₐₚₚₗ=0.5 V) was constantly applied (240 h) to the working electrode with respect to the reference electrode (Ag/AgCl in 3.5M KCl) and counter electrode using a grid powered potentiostat (Interface 1000E, Gamry Multichannel potentiostat, USA). The solar panel (Uxcell 0.5 V 100 mA Poly Mini Solar Cell Panel Module) with the output voltage 0.5 V (Eₐₚₚₗ=0.5 V) was directly connected to the bioreactors for 240 h and the resulting current uptake/electron uptake to the bioreactor was measured with the resistor using ohm's law of electrical current. Electron uptake was collected every 1 min using the Gamry Echem Analyst™ (Gamry Instruments, Warmister, Pa.) software package. At the end of the bioelectrochemical experiment, the samples were immediately collected from the BEC reactors. n-butanol, acetone, and substrates were measured as described above.

Calculations of CCE, Electron Conversion Efficiency, Electrical Energy Conversion Efficiency, and Electron Consumption of n-Butanol, Acetone, CO₂, H₂, and Biomass Biosynthesis.

CCE, electron conversion efficiency, and EECE were calculated by dividing the total carbon/electrons/electrical-energy consumption by the final carbon/electrons/energy content in n-butanol, respectively. To determine carbon consumption, acetate, 3-hydroxybutyrate, or CO₂ consumption was calculated by subtracting the amount in the sample at the end of the experiment from the amount at the beginning of the experiment. Then all the carbon substrate consumptions were converted to moles of carbon, using Eq. (1). The amount of carbon converted to n-butanol was calculated based on the n-butanol production, using Eq. (2). The CCE was calculated using Eqs. (1), (2), and (3) below.

$$C mol substrate = consumed substrate ( \frac{mol}{L} ) * mol of C in 1 mol substrate \qquad Equation 1$$

$$C mol n \text{-butanol} = \frac{n \text{-butanol} ( \frac{g}{L} ) * mol of C in 1 mol n \text{-butanol}}{molecular weight of n \text{-butanol}} \qquad Equation 2$$

$$Carbon conversion efficiency = \frac{C mol n \text{-butanol}}{C mol substrate} * 100 \% \qquad Equation 3$$

The theoretical total number of electrons available from each consumed electron donor was calculated as described below (Eq. (4)). The total available electrons from the complete oxidation of each organic acid were calculated with the assumption that the final oxidation product was CO₂. The inorganic electron donors such as Fe(II) and H₂ release 1 mole e⁻ and 2 moles e⁻ per mole, respectively. Electrons supplied for the photoelectroautotrophy condition were calculated directly from BEC-based experiments wherein the total current uptake was integrated over the operational time. The total electron uptake was used to calculate the electron conversion efficiency to n-butanol because the electrode is the direct electron donor under this growth condition. The number of electrons required for n-butanol production was calculated from the oxidation state of the carbon in each carbon source and n-butanol. TABLE 14 lists the specific oxidation state, and the number of electrons required per mole of n-butanol is listed for all studied sources and n-butanol.

TABLE 14
Specific oxidation state and number of electrons required per
mole of n-butanol.

| | Oxidation | | | |
|---|---|---|---|---|
| | state of | Oxidation | Mole carbon | Electron |
| | carbon in | state of | needed per | needed per |
| | carbon | carbon in n- | mole n- | mole n- |
| Carbon source | source | butanol | butanol | butanol |
| Acetate | 0 | −2 | 4 | 8 |
| 3- | −0.5 | −2 | 4 | 6 |
| hydroxybutyrate | | | | |
| Carbon dioxide | 4 | −2 | 4 | 24 |

To calculate the total available electrons from each substrate, the amount of consumed substrate (in moles) was multiplied by the theoretical total available electrons per mole of the substrate when fully oxidized to CO₂ (Eq. (4)). For photoelectroautotrophy, the total available electron was calculated based on data collected from a data acquisition system (DAQ, Picolog Datalogger). To obtain the electrons required for n-butanol production, the n-butanol production (in moles) was multiplied by the theoretical number of electrons required per mole (Eq. (5)). The conversion efficiency was calculated by dividing the moles of electrons required for n-butanol production by the theoretical total available electrons (Eq. (6)).

$${e}^{-} mol substrate = consumed substrate \text{} ( mol ) * total available electrons in the substrate \qquad Equation 4$$

$${e}^{-} mol n \text{-butanol} = n \text{-butanol} ( mol ) * electrons required to synthesize 1 mol n \text{-butanol} \qquad Equation 5$$

$$Electron conversion efficiency = \frac{{e}^{-} mol n \text{-butanol}}{{e}^{-} mol substrate} * 100 \% \qquad Equation 6$$

Calculation of the EECE to n-butanol was adapted from a previous study. The EECE was calculated by Eq. (7). The charge supplied to the bioelectrochemical platforms was calculated from data collected by DAQ.

$$EECE = \frac{{Δ}_{r} {G}^{0} gain from {CO}_{2} to n \text{-butanol}}{ charge passed through ( C ) * applied voltage ( V )} * 100 \% \qquad Equation 7$$

The Gibbs free energy gains (ΔrG⁰) for n-butanol was calculated similarly with a previous study by reaction 8 and Eq. (9).

$${C}_{4} {H}_{10} O ( l ) + 6 {O}_{2} \to 4 {CO}_{2} ( g ) + 5 {H}_{2} O ( l ) \qquad Equation 8$$

$${Δ}_{r} {{G}^{0}}_{( {C}_{4} {H}_{10} O )} = {Δ}_{f} {H}_{( {C}_{4} {H}_{10} O )}^{0} - 5 * {Δ}_{f} {H}_{( {H}_{2} O )}^{0} - 4 * {Δ}_{f} {H}_{( {O}_{2} )}^{0} - 6 * {Δ}_{f} {H}_{( {O}_{2} )}^{0} \qquad Equation 9$$

$${Δ}_{f} {{H}^{0}}_{( {C}_{4} {H}_{10} O )} = \text{-77.4} \frac{kJ}{mol , Δ {{G}^{0}}_{( {O}_{2} )}} = \text{-394.39} \frac{kJ}{mol , Δ {{G}^{0}}_{( {H}_{2} O )}} = \text{-273.14} \frac{kJ}{mol , Δ {{G}^{0}}_{( {O}_{2} )}} = 0 kJ \text{/mol}$$

Electron consumption of n-butanol, acetone, CO₂, H₂, and biomass biosynthesis, were calculated by the mole of production multiplied by the electrons required for each mole of product. The molar production of n-butanol and acetone was determined by the titer divided by molecular weight. The molar production of CO₂, H₂, was measured by GC-BID. The molar production of biomass was calculated by the OD660 change between T₀ and T_{f} by Eq. (10).

$$Molar production of biomass = \frac{( {OD}_{Tf} - {OD}_{T 0} ) * ( \frac{cell number}{ml} ) * cell weight}{Molecular weight of biomass} \qquad Equation 10$$

Cell number/ml: 8×10⁸ cell/ml/OD, cell weight: 10⁻¹² g/cell, Molecular weight of biomass: 22.426 g/mol.

Determination of Glycogen Content.

TIE-1 cells were grown in freshwater medium with NH₄Cl or under nitrogen-fixing condition (with N₂) supplemented with 10 mM 3-hydroxybutyrate to and OD660 of 1.8 mL of bacterial culture was pelleted and washed three times with ultrapure water and resuspended in 30% (w/v) KOH with for glycogen extraction. Samples were then incubated at 95° C. for 2 h. Glycogen was precipitated by the addition of ice-cold ethanol to a final concentration of 75%. Samples were put on ice for 2 h followed by 10 min. centrifugation 10,000×g at 4° C. The precipitated glycogen was then washed twice with pure ethanol and dried for 20 min. at 60° C. Glycogen samples were resuspended in 250 μL of 100 mM sodium acetate (pH 4.5) and digested with 2 mg/ml amyloglucosidase (Sigma Aldrich A7420) for 2 h at 60° C. Samples were added with infinity glucose hexokinase liquid reagent (Thermo scientific TR1542) at a ratio of 1:150 according to the manufacturer's recommendation and absorbance reading was done at 340 nm.

RNA Extraction, cDNA Synthesis, and RT-qPCR.

To extract RNA for cDNA synthesis and eventually perform RT-qPCR for analyzing the expression level of the individual genes, culture samples (2.5 ml to 15 ml depending on OD660) were taken at the late exponential (Tₘ) or stationary phase (T_{f}). Samples were immediately stabilized with an equal volume of RNAlater (Qiagen, USA). After incubation at room temperature for 10 min, samples were centrifuged at 21,100×g for 3 min. After the supernatant was removed, the pellet was stored at −80° C. before RNA extraction using the Qiagen RNeasy Mini kit (Qiagen, USA), following the manufacturer's protocol. DNA was removed using a Turbo DNA-free Treatment and Removal Kit (Ambion, USA). DNA contamination was ruled out by PCR using the primers listed in TABLE 12 and TABLE 13. Purified RNA samples were then used for cDNA synthesis by an iScript™ cDNA Synthesis Kit (Biorad, USA). The same mass of RNA was added to each cDNA synthesis reaction. The synthesized cDNA was used for RT-qPCR. RTqPCR was performed using the Biorad CFX connect Real-Time System Model #Optics Module A with the following thermal cycling conditions: 95 OC for 3 min, then 30 three-step cycles of 95 OC for 3 s, 60° C. for 3 min, and 65 OC for 5 s, according to the manufacturer's manual. The reaction buffer was iTaq SYBR Green Supermix with ROX (Bio-Rad). The primers used for RT-PCR (listed in TABLE 15) were designed using primer3 software.

TABLE 15
Primers for RT-qPCR.

| Primer Name | Primer Sequence |
|---|---|
| qPCR phaJ F | CCTTTAAGCTGCCGGTGTTC |
| | (SEQ ID NO: 76) |
| qPCR phaJ R | GTGGTGAGGGTGGCGATT |
| | (SEQ ID NO: 77) |
| qPCR ter F | GCGCAACAATATCTGCCTGA |
| | (SEQ ID NO: 78) |
| qPCR ter R | TGATGCGCTTCTTGGTGTAC |
| | (SEQ ID NO: 79) |
| qPCR adhe2 F | ACCTGCTGTACGAGTATCCG |
| | (SEQ ID NO: 80) |
| qPCR adhe2 R | CAGCTTCGGGAAATTGCAGA |
| | (SEQ ID NO: 81) |
| qPCR phaA F | GGATGCCTTCAACGGTTACC |
| | (SEQ ID NO: 82) |
| qPCR phaA R | CGCGAATTCATCCTGCTGAT |
| | (SEQ ID NO: 83) |
| qPCR phaB F | TTTGAATTTCTCCGCTGCCG |
| | (SEQ ID NO: 84) |
| qPCR phaB R | GGTATCGGTGCAGCAATCAG |
| | (SEQ ID NO: 85) |
| TIE-1recAqRT-PCRFor | ATCGGCCAGATCAAGGAAC |
| | (SEQ ID NO: 86) |
| TIE-1recAqRT-PCRRev | GAATTCGACCTGCTTGAACG |
| | (SEQ ID NO: 87) |
| TIE-1clpXqRT-PCRFor | GGAGATCTGCAAGGTTCTCG |
| | (SEQ ID NO: 88) |
| TIE-1clpXqRT-PCRRev | CCGCTTGTAGTGATTGTGGA |
| | (SEQ ID NO: 89) |
| Km qPCR_F | CTCGTCCTGCAGTTCATTCA |
| | (SEQ ID NO: 90) |
| Km qPCR_R | AGACAATCGGCTGCTCTGAT |
| | (SEQ ID NO: 91) |

The primer efficiencies were determined by performing RT-qPCR using different DNA template concentrations. The genes clpX and recA, which have been previously validated as internal standards, were used. The gene code for kanamycin resistance was also used as an internal standard for the plasmid. After RT-qPCR, the data were analyzed using the ΔΔCT method.

NADH/NAD⁺ Measurement.

Wild-type TIE-1 and Nif mutants were grown with either 3-hydroxybutyrate or H₂ and using NH₄Cl as a nitrogen source. Briefly, 1.8 mL cell cultures from the stationary phase (same phase at which samples were taken for measuring n-butanol) was spinned at 21,000×g for 1 min. inside an anaerobic chamber. Then the pellet was resuspended in either 300 μL 0.2M sodium hydroxide (for NADH extraction) or 300 μL 0.2M hydrochloric acid (for NAD⁺ extraction). The resuspension was then incubated at 50° C. for 10 min. and cooled to below 20° C. on an ice block. While vortexing on medium speed, Equal volume 0.1M acid or base was added to neutralize the sample. After spinning at 21,000×g for 5 min, the supernatant was stored in freezer for the following assays. The enzyme cycling assays were then performed on a BioTek Synergy™ HTX 96-well plate reader measuring absorbance at 570. The amount of NADH/NAD⁺ was quantified relative to a standard curve ranging from 0 to 5 μM.

Transmission Electron Microscopy (TEM).

Wild-type TIE-1 and Phb mutants grown with 3-hydroxybutyrate with either N₂ or NH₄Cl as a nitrogen source was used as representative samples for TEM. Briefly, 5 mL planktonic cell suspensions were centrifuged at 6000×g for 5 min. followed by primary fixation by resuspending the cell pellets in 2% formaldehyde and 2.5% glutaraldehyde in 0.05M sodium cacodylate buffer (pH 7.2) for ˜45 min. at room temperature. Cell pellets were agar encapsulated followed by primary fixation for ˜20 min. Polymerized agar was cut into small cubes and were subjected to secondary fixation for ˜5 h followed by acetone dehydration and resin infiltration. Ultrathin sections (˜70 nm) were cut on a Reichert Ultracut UCT ultramicrotome (Leica, Buffalo Grove, Ill., USA), mounted on copper grids (FCFT300-CU-50, Electron Microscopy Sciences, Hatfield, Pa., USA), and counterstained with lead citrate for 8 min. The sample was imaged with a LEO 912 AB Energy Filter Transmission Electron Microscope (Zeiss, Oberkochen, Germany). Images were acquired with iTEM software (ver. 5.2) (Olympus Soft Imaging Solutions GmbH, Germany) with a TRS 2048×2048k slow-scan charge-coupled device (CCD) camera (TRONDLE Restlichtverstarkersysteme, Germany). Each TEM image was acquired at ×10,000 magnification and 1.37 nm pixel resolution.

Viability Analysis of TIE-1 Under Photoelectroautotrophy.

WT TIE-1 was inoculated into the bioelectrochemical reactors described above, with a starting OD of ˜0.3. After 72 h of incubation, the viability of the biofilm attached to the electrode was characterized by imaging the electrode after staining with the LIVE/DEAD® (L7012, Life Technologies) kit. The attached cells were quantified using NISElements AR Analysis 5.11.01 64-bit software. For imaging of the electrode, prior to cutting a piece of the spent electrode, the electrode from the reactor was washed three times with 1×phosphate-buffered saline (PBS) to remove unattached cells. A piece of the spent electrode was then submerged in 1×PBS in a sterile microfuge tube. Prior to imaging, the electrode piece was immersed in LIVE/DEAD® stain (10 μM SYTO9 and 60 μM propidium iodide) kit and incubated for 30 min. in the dark. The electrode sample was then placed in a glass-bottom Petri dish (MatTek Corporation, Ashland, Mass.) containing enough PBS to submerge the sample. Further, it was imaged on a confocal microscope (Nikon A1 inverted confocal microscope), using 555 and 488 nm lasers and a ×100 objective lens (Washington University in St. Louis Biology Department Imaging Facility). Electrode attached cells were quantified by Elements Analysis software using the protocol described below: Briefly, for each reactor, three images were processed. Z-stacks of each image were split into two channels (one for live cells, one for dead cells), the MaxlP was acquired for the combined z-stacks. After GaussLaplace, local contrast and smoothing, and thresholding, and Object Count was performed for each channel based on a defined radius (0.8-5 μm). Then the percentage of live (or dead) cells was calculated by

$$Live ( or Dead ) cell percentage = \frac{number of Live ( or Dead ) cells}{number of total cells} * 100 \%$$

Toxicity Study. WT TIE-1 with an empty vector (pRhokS-2) was used to test the tolerance of TIE-1 for acetone and n-butanol. To test the tolerance, 0, 0.25, 0.5, 1, or 2% n-butanol (v/v), or 0, 0.1, 0.25, 0.5, 1, or 2% acetone (v/v), was added to FW media with acetate (10 mM). Growth was monitored by recording OD660 overtime.

Statistics.

All statistical analyses (two tails Student's t-test) were performed with Python. p-value <0.05 was considered to be significant. For most of the experiments data are from n=3 of biologically independent samples, from each biologically independent samples n=3 technical replications were performed, For photoelectroautotrophy measurements, data are from n=2 of biologically independent samples, from each biologically independent samples n=3 technical replications were performed. For RT-qPCR for photoelectroautotrophy which data are from n=2 of biologically independent samples, from each biologically independent sample n=2 technical replications were performed.

### Example 2: Synthetic Biology and Metabolic Engineering Improves Bioplastic Production by Rhodopseudomonas Palustris Tie-1

The following example describes methods used to generate TIE-1 mutants. Also described herein is that bioplastic production in modified strains are enhanced several fold.

Abstract

The rapid development of genetic tools has enabled complex synthetic biology, which led to improved bioproduction and a deeper understanding of the metabolic pathways in model organisms. However, non-model microorganisms preserve less studied pathways, which might be beneficial for certain bioproduction. This study developed genetic tools for integrating the target gene into the Rhodopseudomonas palustris TIE-1 (TIE-1) genome. This metabolically versatile strain is an excellent host for investigating multiple bioproductions. Among these bioproductions, bioplastics have been widely studied due to their biocompatibility and biodegradability while preserving the advantages of the properties of petroleum-based plastics. To increase the bioplastic production in TIE-1, genes were introduced that could increase bioplastic production. A phage integration system was built to rapidly and efficiently integrate the target gene into the TIE-1 genome. By integrating an extra copy of RuBisCo, the genetically modified TIE-1 showed higher polyhydroxybutyrate production under various conditions. These genetic tools open the door of performing complex synthetic biology in TIE-1 and its closely related organisms.

### INTRODUCTION

The recent improvement of genetic engineering tools has enabled scientists to systematically engineer multiple organisms to produce various value-added chemicals, including biofuels, therapeutic products, food, and bioplastics. In the early years, most of these engineering efforts were focused on the widely used model organisms, such as Escherichia coli and Saccharomyces cerevisiae. This focus is because a wide range of tools is available for their genetic engineering, which led to profoundly investigated physiologies. However, both E. coli and S. cerevisiae are heterotrophs, so they can only use organic carbon as a carbon source carbon. Besides, significant engineering effort is often needed for these model organisms to produce comparable tiers and yield to the native organism. Recent studies have shown numerous advantages of using other microbial chassis beside these model organisms for bioproduction. Thus, more work has focused on expanding the host choice for synthetic biology into the non-model organisms. One of the microbes that has drawn attention during the past decades is Rhodopseudomonas palustris TIE-1 (TIE-1). TIE-1 is a purple non-sulfur bacterium known for its versatile metabolisms, making it a great host for different bioproductions and pathway studies. TIE-1 has four primary metabolisms, chemoautotrophy, photoautotrophy, chemoheterotrophy, and photoheterotrophy. These different metabolisms enable TIE-1 to use a wide variety of carbon sources [carbon dioxide (CO₂) and many organic acids], nitrogen source [ammonium and dinitrogen gas (N₂)] and electron donors [hydrogen (H₂), ferrous iron Fe(II), poised electrode, etc.]. This wide substrate selection enables TIE-1 to produce value-added chemicals, including bioplastic and biofuel, from naturally abundant resources, such as sunlight, CO₂, Fe(II), and N₂⁴. One of the most appealing features of TIE-1 is its ability to gain electrons directly from a poised electrode, which enables the combination of synthetic biology with microbial electrosynthesis (MES). Using a solar panel powered MES, TIE-1 produced n-butanol with just CO₂, N₂, and light (see e.g., Example 1). Thus the production is sustainable and carbon-neutral.

Besides its ability to utilize various substrates for bioproduction, the versatile metabolism also makes TIE-1 an extraordinary host for pathway investigation. The chemoheterotrophic metabolism allows mutant construction, and the versatile metabolism enables the analysis of the specific pathway under specific growth conditions. For example, use of RuBisCo mutants to study the role of the Calvin cycle in electron transport under various growth conditions, pioABC mutants to investigate the mechanism of electron transport. Not only do these studies offer a further understanding of TIE-1's metabolism, but they also open doors for a deeper understanding of other closely related purple non-sulfur bacteria. However, compared to the model organisms, available genetic tools for TIE-1 are quite limited.

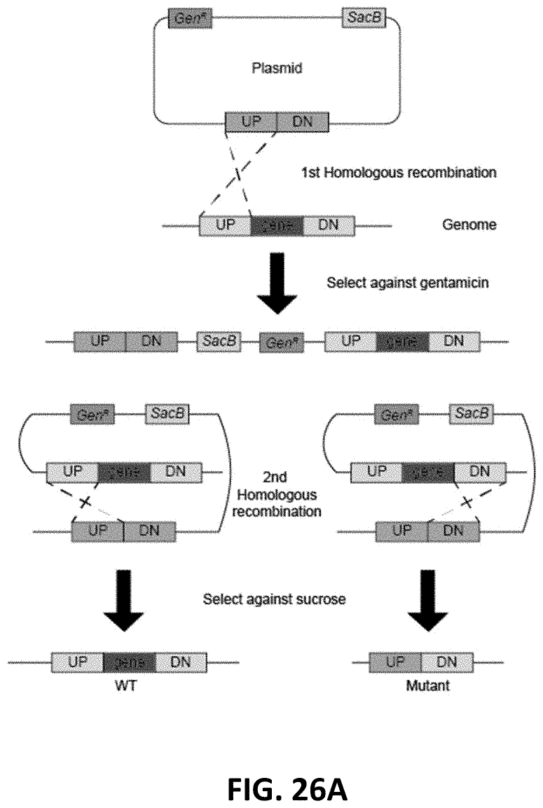

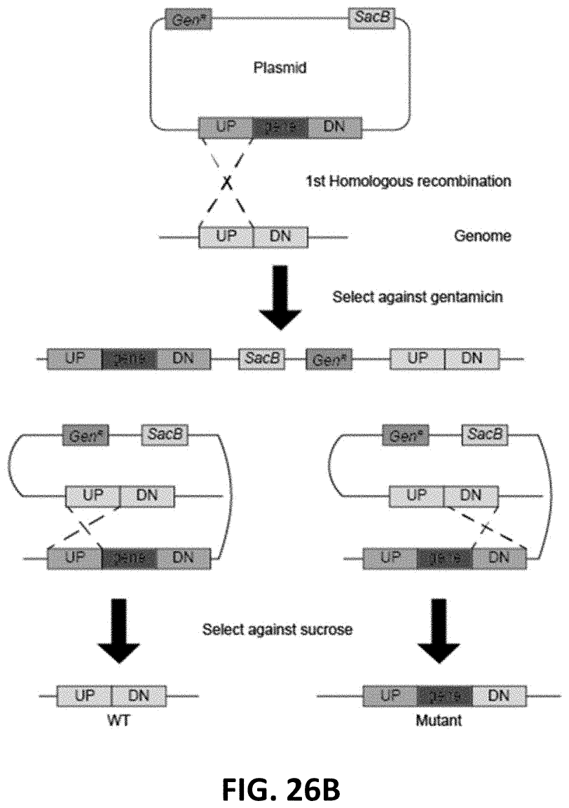

Up to now, most of the genome modification in TIE-1 was accomplished by homologous recombination. As shown in FIG. 26A-FIG. 26B, a suicide plasmid with two 1 kb homologous arms are conjugated to TIE-1. Following the first integration, the integrants are selected against the antibiotic. The integrants are then confirmed by PCR. After the second integration, the mutants are selected against sucrose. Then final PCR steps are used to screen the wildtype (WT) and the integrant. Although this strategy is effective, the homologous recombination takes about two to three weeks, requires three different media, and only has a theoretical efficiency of 50%. Thus, a more rapid, effective genome editing method is needed.

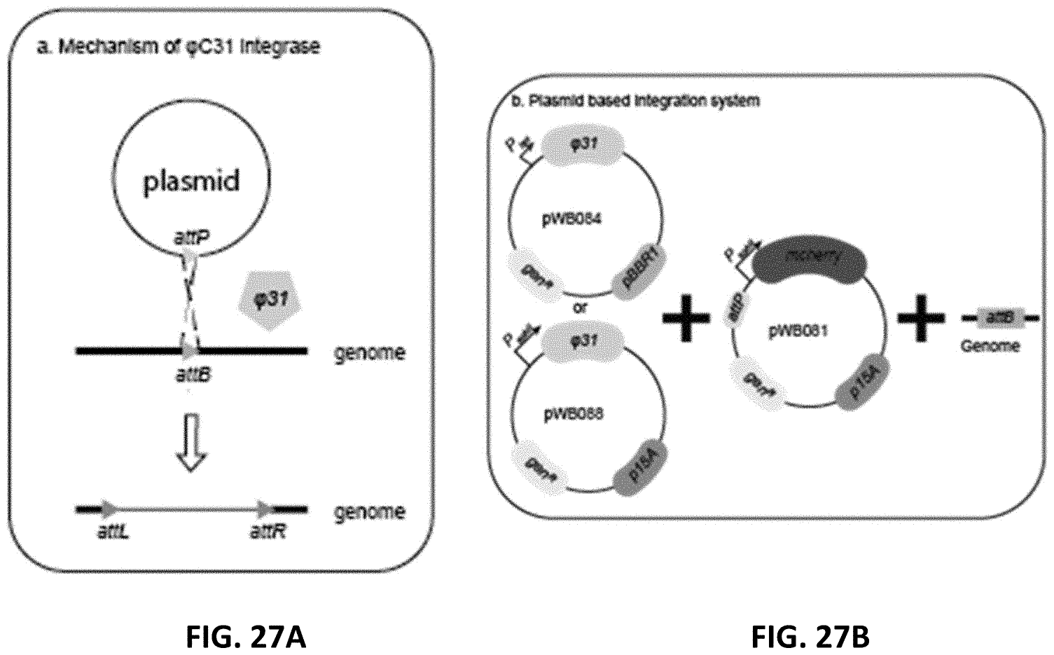

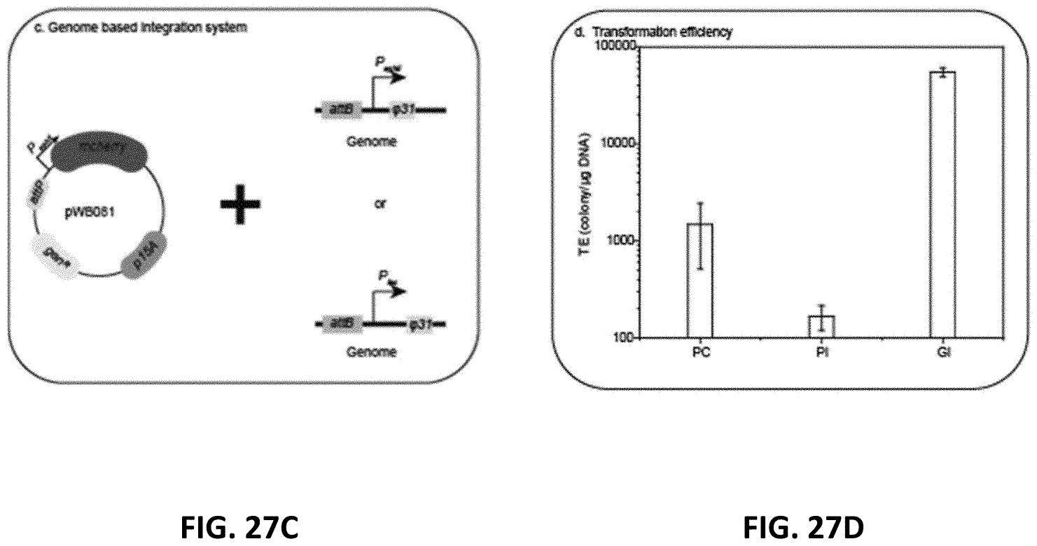

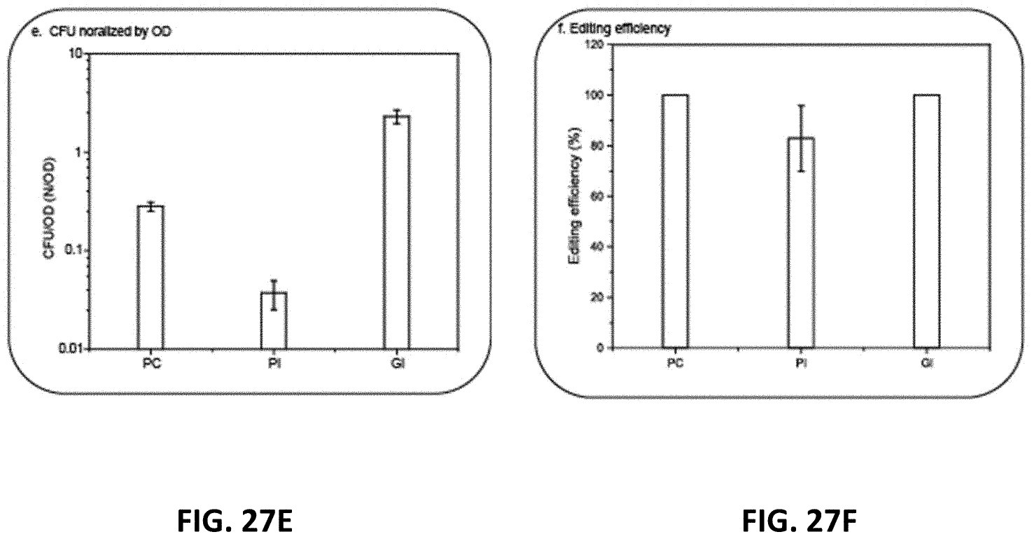

Phage recombination has gained attention for genome engineering in various bacteria, including Methanosarcina and Mycobacterium smegmatis, due to its simple design and high efficiency. Among different phage recombinases, cφC31 has been frequently used because it does not need a helper protein, and it is unidirectional. As shown in FIG. 27A-FIG. 27F, in the presence of φC31, an attB site and an attP site recombine with each other resulting in the insertion of the whole plasmid into the genome. This system is widely used in genome engineering. For example: in Clostridium Ijungdahlii, an entire butyric acid synthesis pathway was integrated into its genome by φC31 recombinase; in Methanosarcina spp. the φC31 recombinase reached genome editing efficiency that is 30 times higher than homologous recombination.

Genome engineering tools have been widely used in producing value-added chemicals. Among these chemicals, bioplastic has been a heated topic in recent years. Petroleum-based plastics are hard to degrade and produce toxic substances during the degradation process. However, life is heavily dependent on these plastics. From 1950 to 2015, approximately 8300 million metric tons of plastic have been produced worldwide. Among all these produced plastics, about 79% was simply buried as landfill or left in the natural environment, which caused many environmental issues. Thus, an alternative way of generating biodegradable plastics is needed. During the past decades, plastics produced by microbes have been proven to be a great substitute.

Synthesized from natural organic materials, bioplastics are biodegradable while preserving the advantage of plastics properties, such as high durability and water resistance. Not only the production process of bioplastic is sustainable, but the bioplastics could also be degraded within 5 to 6 weeks, while petroleum-based plastics can take hundreds to thousands of years to degrade. This short degradation time makes bioplastic easier to recycle. Among all the different kinds of bioplastics, polyhydroxyalkanoate (PHA) is widely studied because not only it is naturally produced in a lot of organisms, but it is also thermoresistant, moldable, and biocompatible. These features allow PHA to be used for drug delivery, reconstructive surgery, bone tissue scaffolding, and other medical applications. However, currently, the high feedstock costs are hindering the production of PHA. Thus a microbe that can use cheap alternative feedstock to produce PHA is needed. TIE-1 can naturally produce polyhydroxybutyrate (PHB), which is one of the widely studied PHA. Thus, herein is described the use of genetic tools to generate knock-in mutants for higher PHB production.

Herein is described the development of a phage recombination system to integrate the target gene in the TIE-1 genome. The effectiveness of these newly engineered genetic tools was tested by generating TIE-1 mutants capable of improving production. By introducing an extra copy of RuBisCo into the TIE-1 genome, an increase in PHB production may be achieved.

Results and Discussion

Phage Integration

As shown in FIG. 27A-FIG. 27F, a φC31 recombinase system needs three parts: attB site, attP site, and the φC31 integrase. Because TIE-1 does not have an attB site in its genome, an attB site was introduced into the genome. The attP site was introduced on suicide plasmid with a constitutively expressed mcherry gene. For the expression of φC31 integrase, the easiest way would be using a plasmid with a temperature-sensitive origin. Unfortunately, there is no known temperature-sensitive plasmid that replicates in TIE-1. As a result, two different systems were built: 1) a plasmid-based system, where the integrase is introduced into TIE-1 by a plasmid (see e.g., FIG. 27B), and 2) a genome-based system, where the integrase is integrated into the TIE-1 genome (see e.g., FIG. 27C). The advantage of the plasmid-based system is mobility, while the edge of the genome-based system is the stability and independence of antibiotics. Both systems were tested with an inducible promoter (P_{lac}) and a strong constitutive promoter (Pₐₚₕₗₗ). In summary, four different designs were created for expression of the φC31 integrase: a) Pₐₚₕₗₗ-driven φC31 integrase on a suicide plasmid; b) P_{lac}-driven φC31 integrase on a self-replicating plasmid; c) Pₐₚₕₗₗ-driven φC31 integrase on TIE-1 genome; and d) P_{lac}-driven φC31 integrase on TIE-1 genome.

The successful integration of mcherry was indicated by visualizing red fluorescence. All the colonies that show red fluorescence signals were tested by a PCR, and 100% of these colonies showed the expected band. After the confirmation of integration, the efficiency of the different systems was evaluated by the transformation efficiency and the integration frequency, defined as the percentage of colonies that have red fluorescence signals among all colonies, are calculated for all three systems.

As shown in FIG. 27D and FIG. 27E, both the electroporation efficiency normalized to OD and to plasmid concentration are higher for the genome-based system. This higher efficiency may be due to the feature that only one plasmid is needed for the system to be functional. Between the two plasmid-based systems, the constitutively expressed φC31 reached higher efficiencies. This higher efficiency may be due to the independence of the inducer. As for the integrating frequency, the genome-based system and the plasmid-based system with the constitutive promoter resulted in editing efficiencies of 100% (see e.g., FIG. 27F). However, the plasmid-based system with the inducible promoter only reached the editing efficiency of about 80% (see e.g., FIG. 27F).

In summary, the genome-based system and constitutive promoter result in higher electroporation efficiency and genome editing efficiency.

Integration of RuBisCo Increased PHB Production.

After testing the phage integration system's functionality by mcherry, genes that could improve bioplastic production were integrated. Previous research has shown that enhanced electron uptake could increase the bioplastic production, and RuBisCo (RuBisCo form I and RuBisCo form II) is essential for electron uptake. Thus an extra copy of RuBisCo form I and RuBisCo form II were integrated into the TIE-1 genome. Two different integrants were made. One only has an extra copy of RuBisCo form I, as it was significantly upregulated during autotrophic conditions. One has both the RuBisCo form I and the RuBisCo form II, as research has shown that RuBisCo form II could complement the ΔRuBisCo from I mutant.

The integrants' PHB production under various conditions was tested. Butyrate was selected as the carbon source because it showed high PHB production in WT TIE-1. H₂ was selected because it is a byproduct of many industries and thus is sustainable. Electroautotrophy was selected because bioproduction can be achieved from just CO₂, N₂, and sunlight (see e.g., Example 1).

Methods

Bacterial Strains, Media, and Growth Conditions

TABLE 16 lists all the strains used in the study.

TABLE 16
Strains used in the study.

| Strains | Relative characteristics |
|---|---|
| pAB415 | Wildtype Rhodopseudomonas palustris TIE-1 |
| WB065 | pAB415 with attB |
| WB068 | pAB415 with φC31 integrase; P_{lac}; laclq; attB |
| WB090 | pAB415 with mcherry; Pₐₚₕₗₗ promoter; fd terminator; attL; attR |
| WB091 | pAB415 with loxp-mcherry-loxp; Pₐₚₕₗₗ promoter; fd terminator; |
| | attL; attR |

Lysogeny broth (LB) was used for the growth of all E. coli strains at 37° C. For genome modification, Rhodopseudomonas palustris TIE-1 was grown in a medium containing 3 g/L yeast extract, 3 g/L peptone, 10 mM MOPS [3-N (morpholino) propanesulphonic acid] (pH 7.0), and 10 mM succinate (YPSMOPS) at 30° C. LB and YPSMOPS agar plates were prepared by addition of 15 g/L agar. When needed, the antibiotic or sucrose was added as indicated.

For PHB production, TIE-1 was grown in anoxic bicarbonate buffered freshwater (FW) medium. All FW medium was prepared under a flow of 34.5 kPa N₂+CO₂ (80%, 20%) and dispensed into sterile anaerobic Balch tubes. The cultures were incubated at 30° C. in an environmental chamber fitted with an infrared LED (880 nm). Under photoheterotrophic conditions, the FW medium was supplemented with sodium butyrate 10 mM for PHB production and 1 mM for growth curve). Under photoautotrophic condition using H₂ as electron donor TIE-1 was grown in FW medium at pH 7.0 and 12 psi of 80% H₂/20% CO₂⁴. For all carbon and electron sources, either ammonium chloride (5.61 mM) or dinitrogen gas (8 psi) was supplied as nitrogen source. All sample manipulations were performed inside an anaerobic chamber with a mixed gas environment of 5% H₂/75% N₂/20% CO₂. When needed, 800 μg/mL gentamycin was added for TIE-1, and 20 μg/mL gentamycin was added for E. coli.

Plasmid Construction

All the plasmids used in this study are listed in TABLE 17.

TABLE 17
Plasmids used in the study

| Plasmid | Relative characteristics |
|---|---|
| pJQ200KS | ori P15A; Gen^{r} |
| pAB314 | pJQ200KS with GlmUSX_UP and GlmUSX_DN |
| pAB356 | Cm^{r} |
| pAB357 | Kan^{r} |
| pAB358 | Tc^{r} |
| pAB359 | Amp^{r} |
| pSRKGm | P_{lac}, laclq; ori pBBR1, Gen^{r} |
| pWB083 | pAB314 with attB |
| pWB081 | pJQ200KS with mcherry, Pₐₚₕₗₗ promoter; fd terminator and |
| | attP |
| pWB084 | pSRKGm with φC31 integrase |
| pWB086 | pAB314 with φC31 integrase; P_{lac}; laclq; attB |
| pWB088 | pJQ200KS with φC31 integrase; Pₐₚₕₗₗ promoter; fd |
| | terminator and attP |

The φC31 attB and attP sequences are obtained from the previously published sequences. The attP sequence was cloned into pJQ200KS, resulting in pWB081. Then the Pₐₚₕₗₗ-mcherry-fd cassette was cloned into pJQ200KS, resulting in pWB081. For the plasmid-based system, the φC31 sequence was cloned to either plasmid pSRKGm or pWB081, resulting in pWB084 and pWB088. For the genome-based system, the phiC31 sequence was cloned to pAB314, resulting in pWB089. The attB sequence was also cloned into pAB314, resulting in pWB083.

Strain Construction

attB site or attB site with an inducible promoter driving phφiC31 was introduced into TIE-1 genome at the glmUSX-recG locus as previously described. Briefly, attB site or attB site with an inducible promoter driving phφiC31 was introduced into pAB314, which has the homologous arms of glmUSX-recG. The integration of the plasmid was selected by gentamicin resistance, and the resulting integrants were screened by PCR. The integrants were then grown non-selectively in the YPSMOPS medium and then plated on YPSMOPS medium with 10% sucrose to select for segregation. Sucrose-resistant colonies were grown on plain YPSMOPS medium and screened by PCR. The final integrant was confirmed by PCR.

TIE-1 Electroporation

To prepare electrocompetent cells, TIE-1 glycerol stock was inoculated in 500 ml YPSMOPS and then incubated at 30° C. After reaching an OD₆₆₀ of 0.5˜0.6, it the culture was centrifuged and washed at 4-degree C. at 4000×g for 10 minutes. After five washes with 10% glycerol, the cell pellet was resuspended in only 2 mL of ice-cold 10% glycerol. This resuspension was aliquoted by 50 μl per sample then saved in −80.

For every electroporation, 0.2 μg of plasmid was added to 50 μl thawed electrocompetent cells. This mixture was then added to 1 mm gap cuvettes and then electroporated at 1.8 kV using gene pulser. After electroporation, the pulsed mixture was added to 2 mL room temperature supper optimum broth (SOB) and grown for 3 hours. 10 μl, 100 μl, and 1000 μl of the culture were then plated on a selective medium. Because these plates will be further imaged by Nikon A1, glass Petri dishes instead of plastic Petri dishes were used.

Imaging of Mcherry

After 4 to 5 days of incubation, the plated electroporated culture was imaged by Nikon. For each plate, an image for the whole plate was captured using a camera through both the bright field and the Texas-red channel with a 10×scope. The colony number of each plate were quantified using NIS-Elements AR Analysis 5.11.01 64-bit software.

Calculations of Transformation Efficiency and CFU Per OD

The phage integration systems were evaluated in two different ways: transformation efficiency with respect to the amount of plasmid added and the CFU per OD. The transformation efficiency is calculated by the following equation:

$$Transformation efficiency = \frac{\begin{matrix}Colony number after electroporation / amount \\ of plasmid DNA ( μ g )\end{matrix}}{\begin{matrix}volume plated on the plate after electroporation / total \\ volume for out growth\end{matrix}}$$

The CFU per OD is calculated by the following equation:

$$CFU per OD = \frac{\begin{matrix}Colony number after electroporation / amount \\ plated on the plate after electroporation\end{matrix}}{\frac{\begin{matrix}C olony number on YPSMOPS plate / volume \\ plated on the YPSMOPS plate\end{matrix}}{cell number per OD}}$$

The colony number on the YPSMOPS plate was determined by plating unelectroporated electrocompetent cells on the YPSMOPS plate with series dilution. Cell number per OD is considered as 8*10⁸ cell/ml.

PHB Measurement

A 10 mL bacterial sample was pelleted at 8000 xg for 10 min and stored at −80° C. until PHB extraction and analysis were performed. 1 mL of water (LC-MS grade) and 600 μL of methanol (HPLC grade) were added to arrest metabolic activity of TIE-1. 10 mg/mL of poly[(R)-3-hydroxybutyric acid] (Sigma-Aldrich, USA) was used as a PHB standard. Extraction of PHB was followed by its conversion to crotonic acid. The concentration of crotonic acid was measured using an Agilent Technologies 6420 Triple Quad LC/MS as follows: using Hypercarb column, particle 5 μm, 100×2.1 mm (Thermo Fisher Scientific, USA) as stationary phase; water with 0.1% (v/v) formic acid as phase A; acetonitrile and 1% (v/v) formic acid as phase B. The injection volume was 5 μL; the flow rate was set at 500 μL min-1; the column temperature was set at 15° C. and the gas temperature was 300° C. PHB was detected as crotonic acid with mass to charge ratio (m/z)=87 which was normalized to bacterial cell number.

1-30. (canceled)

31. A plasmid-based method for integrating a target gene into a genome of Rhodopseudomonas palustris TIE-1 (TIE-1), the method comprising:
introducing an attB site into the genome of TIE-1;
providing a suicide plasmid including an attP site and a target gene;
providing a plasmid including a φC31 integrase; and
expressing the φC31 integrase in the presence of the attB site and the attP site such that the target gene is integrated into the genome.

32. The method of claim 31, wherein the integrated target gene increases bioplastic production in TIE-1.

33. The method of claim 32, wherein the bioplastic is a polyhydroxyalkanoate (PHA).

34. The method of claim 33, wherein the PHA is polyhydroxybutyrate (PHB).

35. The method of claim 31, wherein the target gene comprises at least one copy of RuBisCo.

36. The method of claim 31, wherein providing a plasmid including a φC31 integrase comprises providing a suicide plasmid including the φC31 integrase with a constitutive promoter.

37. The method of claim 31, wherein providing a plasmid including a φC31 integrase comprises providing a self-replicating plasmid including the φC31 integrase with an inducible promoter.

38. A genome-based method for integrating a target gene into a genome of Rhodopseudomonas palustris TIE-1 (TIE-1), the method comprising:
introducing an attB site into the genome of TIE-1;
providing a suicide plasmid including an attP site and a target gene;
introducing a φC31 integrase into the genome of TIE-1; and
expressing the φC31 integrase in the presence of the attB site and the attP site such that the target gene is integrated into the genome.

39. The method of claim 38, wherein the integrated target gene increases bioplastic production in TIE-1.

40. The method of claim 39, wherein the bioplastic is a polyhydroxyalkanoate (PHA).

41. The method of claim 40, wherein the PHA is polyhydroxybutyrate (PHB).

42. The method of claim 38, wherein the target gene comprises at least one copy of RuBisCo.

43. The method of claim 38, wherein introducing a φC31 integrase into the genome of TIE-1 comprises introducing the φC31 integrase with a constitutive promoter into the genome of TIE-1.

44. The method of claim 38, wherein introducing a φC31 integrase into the genome of TIE-1 comprises introducing the φC31 integrase with an inducible promoter into the genome of TIE-1.

45. A transgenic Rhodopseudomonas palustris TIE-1 (TIE-1) comprising at least one copy of RuBisCo integrated into a genome of TIE-1.

46. The transgenic TIE-1 of claim 45, wherein the transgenic TIE-1 overproduces polyhydroxybutyrate (PHB) compared to a TIE-1 without the at least one copy of RuBisCo integrated into the genome.

47. A transgenic Rhodopseudomonas palustris TIE-1 (TIE-1) comprising a gene cassette including phaJ, ter, and adhE2.

48. The transgenic TIE-1 of claim 47, wherein the transgenic TIE-1 is a nitrogenase knockout mutant.

49. The transgenic TIE-1 of claim 48, wherein the gene cassette further includes phaA and phaB.

50. The transgenic TIE-1 of claim 47, wherein the transgenic TIE-1 overproduces n-butanol compared to a TIE-1 without the gene cassette.
